(19) United States
(12) Patent Application Publication
(10) Pub. No.: US 2026/0079118 A1
(43) Pub. Date: Mar. 19, 2026

(54) SURFACE CHARACTERIZING DEVICE SYSTEM

(71) Applicant: Hempel A/S, Kongens Lyngby (DK)

(72) Inventors: Stefan Urth Nielsen, Kongens Lyngby (DK); Lena Marijke Wenzel, Kongens Lyngby (DK); Mark Terrell Sutton, Kongens Lyngby (DK); Kristian Vingaard Petersen, Kongens Lyngby (DK)

(21) Appl. No.: 19/109,193

(22) PCT Filed: Sep. 6, 2023
(86) PCT No.: PCT/DK2023/050215

§ 371 Date: Mar. 6, 2025

(30) Foreign Application Priority Data
Sep. 6, 2022 (DK) PA 2022 70434

(51) Int. Cl. G01N 21/94; G01N 21/3563; G01N 21/552; G06N 3/0464; G06N 3/09; G06N 3/096

(52) U.S. Cl. CPC G01N 21/94; G01N 21/3563; G01N 21/552; G06N 3/0464; G06N 3/09; G06N 3/096

(57) ABSTRACT

A method and a device system for measuring a characteristic of a surface comprising a cured coat is disclosed. The device system, which is portable and battery powered, comprises a sensor configured to acquire a measurement of a characteristic the surface, and a processor and memory are configured to establish a representation of the measurement. A display visual output device, such as an LED or display, is configured to indicate a measurement or connection status, while a user interface is configured to control measurement. A communication module establishes a communication channel with a cloud and transmit the measurement representation via the communication channel. The device system also comprises a measurement buffer to temporarily store the measurement representation when the communication module is prevented from establishing the communication channel. The temporarily stored representation are transmitted upon reestablishment of the communication channel. The device system may be a measurement device together with a smart device.

### CROSS REFERENCE TO RELATED PATENT APPLICATIONS

This application is a U.S. National Phase Application of International Application No. PCT/DK2023/050215, filed Sep. 6, 2023, which claims priority to Danish Patent Application No. PA 2022 70434, filed on Sep. 6, 2022, all of which are hereby incorporated by reference in their entirety.

### FIELD OF THE INVENTION

The present invention relates to device systems for characterizing surfaces comprising cured coats, in particular to solutions for improving the robustness of using such devices in the field.

### BACKGROUND OF THE INVENTION

Analysing surfaces in laboratories or under controlled circumstances is well-known. Tasks can be planned, and the equipment be set up and tested for the specific task beforehand. When unexpected events challenge the conduct or quality of a task in a laboratory it can usually be postponed until the challenges have been resolved.

When doing surface analysis in the field instead of a laboratory, some of the above premises may be less clear. In particular for onsite assessment of coated structures in for example the industrial or marine categories, where physical access itself may be challenging and resource demanding, it may not be possible to plan ahead and prepare in the same way as in laboratories, and it may be impossible or disproportionate with respect to lost resources to reschedule in order to overcome unforeseen challenges.

### SUMMARY OF THE INVENTION

The inventors have identified the above-mentioned problems and challenges related to onsite assessment of cured coat, and subsequently made the below-described invention which may provide a feasible solution for cured coat assessment in for example remote or hardly accessible environments.

The invention relates to a surface characterizing device system, comprising at least one sensor configured to acquire a measurement of at least one characteristic of a surface comprising a cured coat; a processor and memory configured to establish a representation of said measurement; a visual output device, such as an LED or display, configured to indicate at least a measurement or connection status; a user interface configured to control measurement; and a communication module configured to establish a communication channel with a cloud and transmit said representation of said measurement via said communication channel; wherein said surface characterizing device system is a portable system and being battery powered; wherein said surface characterizing device system comprises a measurement buffer and is configured to temporarily store said representation of said measurement in said measurement buffer when said communication module is prevented from establishing said communication channel and transmit said temporarily stored representation upon reestablishment of said communication channel.

A specialized surface characterizing device system according to the invention, which is portable and battery powered, and which has communication means for transmitting measurements to the cloud, is particularly advantageous for onsite and in field surface measurements.

The invention may also specifically target some of the challenges related to onsite assessment of coated structures in for example the industrial or marine categories, where physical access itself may be challenging and resource demanding, and where it may be impossible or disproportionate with respect to lost resources to reschedule. In particular, the invention is implemented so that it is not necessary to revisit a measurement site just because the connection to the cloud is momentarily lost when the measurements are performed. This may be advantageous when performing measurements in remote places, for example an offshore wind farm, where coverage of common communication networks may be lacking, and/or in enclosures, for example a silo or cargo or ballast tank, where wireless communication is shielded by thick concrete or steel walls. By means of a measurement buffer, this problem may be solved according to the invention, by temporarily storing measurements until within coverage or until re-establishment of the communication channel. The solution of the present invention may thereby be highly advantageous over alternatives, which are not even always possible, such as implementing highly expensive satellite communication in the measuring equipment to improve remote coverage, running long network cables to the outside to overcome the shielding problem, coming back to the place at a later time with different equipment, or implementing an SD memory card for local storing which requires the user to remember to move the measurements from the SD card to the intended storage when back at a PC, and with the risk of the SD card being lost or deleted by mistake before it get copied.

The invention may further implement advantageous solutions for managing the measurement buffer storage, and for performing surface measurements onsite by a portable system.

A large variety of structures made e.g. of steel or concrete are covered on at least a part of the surface with a coating system comprising one or more layers of a “cured coat” serving different purposes, inter alia protection against atmospheric degradation including corrosion, fading, and UV-caused degradation etc., reduction of fouling, abrasion resistance, chemical resistance, prevention of reflection, or simply providing an aesthetic appearance. Under ideal conditions, the cured coat exhibits a predefined, intended property, e.g. a specific level of protection against absorption of air, water or corrosive substances, and it therefore preserves the intended condition of the structure.

When used herein, the term “coated structure” refers to a structure comprising a base and a coating system applied on a surface of the base. The base may e.g. comprise concrete, metal or alloys such as steel, iron or aluminum, or composites such as reinforced concrete, composite wood such as plywood, reinforced plastics, such as fibre-reinforced polymer or fiberglass, ceramic matrix composites, metal matrix composites, etc. The base may for example form an asset such as a building, a ship hull, a tank, a bridge, a wind turbine tower, a chimney, an industrial facility, etc. The coating system may comprise one or more layers of cured coat, e.g. a first layer of cured coat and a second layer of cured coat obtained by application of identical and/or different coating compositions. Each layer comprises opposite inner and outer surfaces, the inner surfaces being joined in a coating system interface. The outer surface of the first layer may e.g. be joined to the base in the base interface.

When used herein, the term “surface comprising a cured coat” is understood broadly as the combined substances forming the outer surface of the coated structure, thereby at least comprising the cured coat, but also possibly comprising any subsequently desired or undesired treatment or contaminants, e.g. deposits or residues, water absorption, corrosive substances, biologic or inorganic material, etc.

When used herein, a “cured coat” indicates a coat obtained by applying a coating layer (i.e. a layer of a coating composition) to a surface of a base structure and allowing the composition to cure. A cured coat may be obtained from application of one or more layers of a coating composition to obtain a desired thickness of the cured coat. A coating layer may be considered to have become a cured coat for example when the curing reaction, such as crosslinking by amine-epoxide reaction or isocyanate-hydroxyl reaction, etc., and/or solvent evaporation, is about 90% complete, such as more than about 95% complete, preferably about 99% complete. A coating layer may be considered a cured coat when the coating have passed its vitrification point. A coating layer may be considered a cured coat after leaving for at least 7 days at a temperature of 20-27° C. after application of the coating composition to the surface, or after leaving for at least 14 days at a temperature of 0-20° C. after application of the coating composition to the surface. The term “coating composition” in the context of the invention encompasses both one-component and two-component compositions.

The term “cured coat” is used as a general term covering all types of cured paint coats curing such as for example curing obtained by crosslinking of a binder and a curing agent in a two-component coating system, curing obtained by evaporation of organic solvent or water (also called physically drying) with or without heating, and curing obtained by other means such as by radiation curing.

A “sensor” for measuring a characteristic of a surface comprising a cured coat, may be any sensor suitable for portable, battery powered use. The sensor may also comprise several sensors, possibly of different type, technology and aim. It may for example be advantageous to implement a spectroscopy sensor to identify ID profiles or individual constituents of the cured coat and possibly contaminants, such as for example attenuated total reflection ATR spectroscopy or hyperspectral imaging spectroscopy. It may also or in addition be advantageous to implement a visible spectrum camera to evaluate surface characteristics such as cracks, scratches, blisters, etc. The sensor may also comprise supplementary sensors to enhance or validate the results from the main sensors, or aid the operator in making suitable measurements.

A “visual output device” for indicating at least a “measurement or connection status” may be anything from a simple LED with different colours or blink codes indicating different statuses, to a high-resolution touch display for providing detailed results and status to the operator. Likewise, a “user interface” may comprise simple buttons or knobs for turning the device on and off, or starting a measurement, or it may use a touch display for receiving text and touch gestures.

The term “cloud” is understood by the skilled person in the present context as referring to a cloud computing environment, i.e. computing resources and/or data storage resources that are generally available via the Internet, preferably subject to encryption, authentication or other data security measures. The cloud may be a public or private cloud or any variation thereof, and may be geographically located physically at a specific data centre and/or geographically distributed and/or mirrored among several data centres and/or edge computing devices. The “communication module” may be any suitable communication means for communicating with a cloud computing system or data centre, for example via the Internet, and depending on available gateways, for example GSM, LTE, 4G, 5G or other mobile data interface, Wi-Fi when an access point is available, for example established using Wi-Fi sharing by a nearby smart device SD, e.g. smartphone, etc. The communication module may also establish the communication channel through a local gateway, e.g. a nearby smart device SD, such as a smartphone, tablet computer or laptop, to which it may communicate by for example Wi-Fi, USB, Bluetooth, BLE, NFC, ultrawideband UWB, etc. In an embodiment the communication module comprises a wired network interface, e.g. for establishing the first part of the communication channel by means of, e.g., Ethernet or USB connection.

The term “measurement buffer” is understood by the skilled person in the present context as referring to a local data storage for temporarily storing the representations of measurements while the communication module is prevented from transmitting them to the cloud. The measurement buffer may comprise any kind of data storage resources suitable for writing and reading, and is preferably of a type that retains data even when power is removed so that non-transmitted representations of measurements can be kept in the measurement buffer also for longer periods, such as hours or days, of non-establishment of the communication channel where the surface characterizing device system is powered off in the meantime. The measurement buffer may be part of the memory of the surface characterizing device system, for example when the device memory comprises both volatile memory, e.g. RAM, and non-volatile memory, e.g. flash memory or an SSD. The measurement buffer is dimensioned to accommodate a representation of at least one measurement, but is preferably sufficiently large to accommodate representations of several measurements, for example 2, 3, 4, 8, 16, 64, 100, 256, 500 or more measurements, which allows the device to make several measurements while being offline, without losing data. The measurement buffer may be configured according to a first-in-first-out FIFO principle, so that upon connection the oldest measurements are transmitted first, or any other buffer or queue principle may be implemented. When the measurement buffer is implemented as part of the device memory, i.e. sharing memory resources with other storage demands of the device, e.g. for firmware, software, processing or display, the measurement buffer may have a dynamic size within the limits of the overall memory resources of the device so that the measurement buffer can grow or shrink according to need.

The surface characterizing device system can be a single unit or be distributed into two or more units. The surface characterizing device system may for example be distributed into a sensor unit at least implementing the at least one sensor, and a communication module unit implementing at least the communication module, and wherein the processor, memory, visual output device, user interface and measurement buffer may be implemented in either or both units in various embodiments. In another example the surface characterizing device system is distributed into a sensor unit at least implementing the at least one sensor, and a user interface unit implementing at least the visual output device and the user interface, and wherein the processor, memory, communication module and measurement buffer may be implemented in either or both units in various embodiments. When the surface characterizing device system consists of two or more units, each unit comprises an inter-unit communication module to allow internal communication in the device between the separate units. The unit wherein the communication module of the surface characterizing device system is implemented, may use the same communication module also as inter-unit communication module, or comprise two different communication modules. The communication technology between separate units of the surface characterizing device system may preferably be selected from low-power local area communication technologies such as BLE, Bluetooth, ZigBee, ANT, Wi-Fi, NFC, UWB, etc. For example, the surface characterizing device system may comprise in a first portable battery powered separate unit a processor, memory, a status indicator and an inter-unit communication module, and in a second separate unit comprise in a second portable battery powered separate unit a processor, memory, a display, a user interface, a measurement buffer, a communication module for cloud communication and an inter-unit communication module for communicating with the first separate unit. The second portable battery powered separate unit may for example be a smart device SD, such as a smartphone, a tablet computer or a laptop computer configured with suitable software to implement the functionality of the invention. In another example the measurement buffer is in the first separate device together with the sensor, to allow temporary storage of representations of measurements even if the inter-unit communication module is prevented from establishing communication with the second separate module. In another example the second portable battery powered separate unit may for example be a docking station comprising the communication module and a removable wired inter-unit communication module, e.g. a USB interface, and the measurement buffer being implemented in the first separate unit together with the sensor.

In an embodiment said temporarily stored representation is a training input measurement or a labelled training input measurement for a training system configured to train a classification model for characterizing a surface comprising a cured coat.

In an embodiment said temporarily stored representation is an input measurement for a classification system configured to classify said surface comprising a cured coat.

In an embodiment the surface characterizing device system is a distributed system comprising a portable measurement device and portable smart device, such as a smart phone, tablet computer or laptop computer; wherein the measurement device comprises at least one of said at least one sensor; wherein the smart device comprises at least said communication module configured to establish said communication channel with said cloud; and wherein the measurement device is communicatively coupled to said smart device, such as by Bluetooth, Wi-Fi, or a USB-cable, to transfer said measurement or said representation.

The measurement device part of the surface characterizing device system may preferably comprise specific sensor technology suitable for the intended surface characterization when such is not usually available in generic smart devices. For example, a measurement device specifically configured for ATR spectroscopy in the wavelength range of 5,500 nm to 8,000 nm for identifying coat characteristics such as binder systems or coat product category, may be advantageous because such a sensor is typically not available in generic smart devices. The smart device, on the other hand, may provide a high-quality user interface the users are already familiar with, several communication technologies at hand, and efficient processing and memory capabilities. The smart device may also be useful for its built-in generic sensors such as visual spectrum camera, GPS, accelerometer, etc.

Such a two-part surface characterizing device system with inter-communication between the measurement device and the smart device, and a measurement buffer for storing representations of measurements until they can be transmitted by the smart device to the cloud, may advantageously implement metadata synchronization to ensure that the representation of a measurement which is transmitted to the cloud after temporary storing in a measurement buffer for an arbitrary duration, is assigned with metadata corresponding to the time and geolocation when the measurement by the measurement device took place, and not when the representation was transmitted. Particularly, when a measurement is made, the surface characterizing device system should preferably acquire all the circumstantial metadata that is going to be assigned to the representation of the measurement, such as one or more of timestamp, geolocation, operator, asset identification, etc., because such information may have changed or may no longer be available when the representation of the measurement is eventually transmitted to the cloud after a shorter or longer duration of being stored in the measurement buffer. For instance, both the time and geolocation will typically have changed if the buffering is required due to measuring inside an electromagnetically shielding structure, and the communication becomes available when the operator at a later time has moved the system out of the radio-hindering structure.

In an embodiment the measurement buffer is comprised in the smart device; and wherein the user interface, visual output device, processor and memory, respectively, are comprised in the smart device and/or in the measurement device.

In an embodiment the processor and memory configured to establish said representation to be transmitted via said communication channel are comprised in the smart device, and wherein the measurement device is configured to transfer said measurement to said smart device via said communicative coupling.

In an embodiment the smart device comprises said visual output device and said user interface, such as combined in a touch screen.

In an embodiment the visual output device and/or the user interface, respectively, are distributed between the measurement device and the smart device, such as one or more status LEDs and buttons on the measurement device and a touch screen on the smart device.

In an embodiment the at least one sensor is at least two sensors and wherein said smart device comprises at least one of said at least two sensors.

The surface characterizing device system may advantageously combine measurements from the specific one or more sensors of the measurement device, such as spectroscopy or dry film thickness, with measurements from more generic sensors of the smart device, e.g. camera or GPS.

In an embodiment the smart device is configured to assign to the representation of said measurement further data from a sensor of said smart device and/or training input labels and/or metadata obtained by the smart device, for example relating to geolocation, weather, temperature, humidity, light, operator, surface, asset type, asset identification, etc., or information about periods where said communication module has been prevented from establishing said communication channel, i.e. when the surface characterizing device system has been online and offline, respectively.

In an embodiment the surface characterizing device system is configured to perform metadata synchronization between the time and/or geolocation of said measurement and any metadata included in the representation being transmitted via said communication channel after said temporary storing in said measurement buffer.

In an embodiment said measurement buffer is configured to temporarily store at least two, such as at least 5, for example at least 10, 25 or 50, of said representations of said measurements.

In an embodiment said measurement buffer is configured to delete said temporarily stored representations of said measurements when they have been transmitted upon reestablishment of said communication channel and/or upon request from an operator of said surface characterizing device system.

In an embodiment said surface characterizing device system is configured to indicate by said visual output device a status of said measurement buffer, such as a number of temporarily stored representations, a number of remaining free storage positions, an indication that the buffer is full, or whether the measurement buffer is active due to establishment of said communication channel being prevented.

In an embodiment said surface characterizing device system is configured to perform pre-processing of said representation before said temporarily storing in said measurement buffer.

In an embodiment said surface characterizing device system is configured to encrypt and/or compress said representation before said temporarily storing in said measurement buffer, or when transmitting said temporarily stored representations via said communication channel.

In an embodiment the communication module is a wireless communication module, such as a Wi-Fi, GSM, LTE, 4G or 5G communication module.

In an embodiment said sensor is configured for making spectroscopy measurements.

“Spectroscopy measurements” may in the present context refer to any measurement relating to how matter, such as a surface comprising a cured coat, interacts with electromagnetic radiation in the form of absorption events between the surface and the radiation, as a function of the wavelength of the radiation. In other words, spectroscopy measurements may involve measuring electromagnetic radiation from a surface comprising a cured coat to determine the absorbance, reflection, radiation or transmission over a wavelength spectrum, possibly after exposing the surface to energy, such as electromagnetic radiation, for example infrared, visible or ultraviolet light.

In the context of the invention, particular mention is made of infrared spectroscopy, also referred to as IR spectroscopy, where the measured spectrum involves a subset of the infrared spectrum, and where the surface is typically exposed to infrared radiation and the absorbance or reflectance of the surface during the exposure is measured in the selected spectrum. Infrared spectroscopy may for example be useful in identifying functional groups of organic molecules, as the absorption of infrared light by organic molecules causes molecular vibrations at frequencies unique to the individual functional groups. Different specific vibration, bendings, and stretching modes of the chemical bonds of molecules affect the absorbance of various infrared wavelengths by the surface. Hence, different chemical bonds correspond to specific infrared spectral bands as they have different molecular vibrational energy modes. Thus, in the infrared spectrum the chemical bonds that the molecules are made of gives a specific “code” or combination of wavelength absorbance. For example, a bisphenol epoxy resin would among others contain infrared absorbance peaks from the vibrations of the epoxide C—O from the carbon-oxygen bonds, C—H (sp³) from the aliphatic carbon-hydrogen bonds, C—H (sp²) from the aromatic carbon-hydrogen bonds, and C═C from the aromatic carbon-carbon double bonds. Therefore, IR spectroscopy may be useful to measure which bonds, i.e. representative of molecules, are present in the measured material, and by several measurements over time, also monitor the disappearance and formation of new of bonds, i.e. different molecules, to determine if the material is changing and how it is changing. The analysis of IR spectroscopy measurements may involve identifying functional groups by considering wavelengths of characteristic peaks, or may rely on an ID profile approach where patterns of several less characteristic bumps and dimples are considered, or a combination of functional group identification and an ID profile approach.

Spectroscopy in visible or ultraviolet spectra, or other electromagnetic spectra, may in various embodiments in addition or alternatively be useful for characterizing the surface comprising a cured coat, for example to analyze pigments (visible spectrum), atoms (ultraviolet or x-ray), etc.

Waves at various wavelengths of electromagnetic radiation or absorbance may be referred to herein by their wavelength λ defined as the spatial period of the wave, i.e. a distance in units of e.g. mm, μm or nm; their frequency f defined the temporal frequency, i.e. a number of periods per second, e.g. in units of GHz or THz; or their wavenumber \tilde{ν} defined as the spatial frequency of the wave, i.e. the number of periods per distance in units of e.g. cm⁻¹. Regardless which of these properties and units are referred to, the other two can be determined by their relationship: \tilde{ν}=1/λ=f/c, where c denotes the speed of light. For example, an infrared wave in the fingerprint region may have a wavelength λ of 10,000 nm, corresponding to a wavenumber \tilde{ν} of 1,000 cm⁻¹, again corresponding in vacuum to a frequency f of about 30 THz. For example, an absorbance spectrum indicated by absorbance as a function of wavenumbers in the interval from 1250 cm⁻¹ to about 952 cm⁻¹ corresponds to the wavelength spectrum of 8,000 nm to 10,500 nm and the frequency spectrum of about 29 THz to about 38 THz. Another example would be an absorbance spectrum indicated by absorbance as a function of wavenumbers in the interval from about 1818 cm⁻¹ to 1250 cm⁻¹ corresponding to the wavelength spectrum of 5,500 nm to 8,000 nm and the frequency spectrum of about 55 THz to about 38 THz. A significant portion of bonds from the binder systems have peaks within this range.

The infrared spectrum, IR spectrum, may be defined as electromagnetic radiation with wavelengths from about 750 nm to 1 mm, corresponding to wavenumbers from about 13,333 cm⁻¹ to 10 cm¹. The term “near infrared” or NIR as used herein refers to the shorter wavelengths within the IR spectrum, closest to visible light, for example from about 14,000 cm⁻¹ to 4,000 cm⁻¹, or from 700 nm to 2,500 nm. The term “mid infrared” or MIR as used herein refers to the wavelengths from 2,500 nm to 25,000 nm, corresponding to wavenumbers from 4000 cm⁻¹ to 400 cm⁻¹. The MIR range is subdivided into short wavelength IR, or SWIR, from 1400-3000 nm, medium wavelength IR, or MWIR, from 3,000-8,000 nm, and long wavelength IR, or LWIR, from 8,000-15,000 nm, i.e. from 1,250 to about 670 cm⁻¹. “Far infrared”, or FIR, as used herein, covers the range from 15,000 nm to 1,000,000 nm. A functional group region of the IR spectrum for spectroscopy may be considered the range of 2,500 nm to about 7,000 nm, and an IR fingerprint region being the range of about 7,000 nm to 20,000 nm.

Spectroscopy measurements may be obtained by any suitable spectroscopy technique known by the person skilled in the field of spectroscopy. Some suitable techniques for spectroscopy of surfaces may involve attenuated total reflectance spectroscopy, or ATR spectroscopy, where IR light is transmitted through a crystal with internal reflections before arriving at an IR sensor. Reflection on a surface of the crystal which is in physical contact with an external surface such as cured coat is attenuated by absorbance in the cured coat. The ATR crystal may for example be implemented in a Fourier-transform infrared spectrometer, or FTIR spectrometer, to obtain the degree of absorbance in the cured coat as a function of wavenumber. A suitable sensor may for example be a tuneable pyroelectric detector with a tuneable Fabry-Perot filter to measure a range of wavelength sequentially. Such sensors may be designed for a limited range of infrared wavelengths, for example 5,500 nm to 8,000 nm, or 8,000 nm to 10,500. An embodiment implements two or more IR sensors to provide spectroscopy measurements in a broader or overlapping range of wavelengths. Two or more IR sensors may optionally share the same ATR crystal and possibly the same IR emitter, depending on the emitter's wavelength range. With ATR spectroscopy, the measurement is influenced to a depth into the material to be tested of about 1 μm to 2.5 μm depending on the wavelengths. Another suitable spectroscopy technique to obtain spectroscopy measurements may involve hyperspectral imaging HSI, imaging spectroscopy or 3D spectroscopy, where absorbance of the surface is measured from a distance as a function of wavenumber and spatial position, much like a digital color camera only with typically many more than three distinctive wavenumbers, thereby obtaining a so-called hyperspectral cube. The skilled person in the field of spectroscopy is able to implement any of ATR spectroscopy and HSI, or other spectroscopy measurement implementations according to his knowledge for use with the invention.

In an embodiment the sensor is configured to obtain the spectroscopy measurements by attenuated total reflectance ATR spectroscopy at wavelengths in the range of 5,500 nm to 8,000 nm

In an embodiment said surface characterizing device system comprises at least one infrared emitter, at least one prism and at least one sensor configured for attenuated total reflection spectroscopy of said surface comprising a cured coat to obtain a spectroscopy measurement, such as single-bounce attenuated total reflection spectroscopy.

In various embodiments said at least one infrared emitter, said at least one prism and said at least one sensor are configured to perform spectroscopy in the long wavelength infrared LWIR range, such as in the range of 8,000 nm to 10,500 nm, in the medium wavelength infrared MWIR range, such as in the range of 5,500 to 8,000 nm, or a combination thereof, such as the range of 5,500 nm to 10,500 nm. In embodiments using infrared emitters and/or sensors with limited wavelength range, a broader range or combination of ranges may be achieved by implementing two or more infrared emitters and/or sensors, possibly sharing the same prism, or implementing a prism for each sensor.

In an embodiment said sensor is a tuneable pyroelectric detector configured to obtain measurements at different wavelengths.

In an embodiment said surface characterizing device system comprises a multispectral or hyperspectral imaging camera for performing spectroscopy of said surface comprising a cured coat to obtain a spectroscopy measurement, preferably in the near infrared NIR range, such as in the range of 930 nm to 2500 nm, or the mid infrared MIR range.

In an embodiment said surface characterizing device system comprises a pressure sensor and/or an accelerometer sensor.

Thereby it may be possible to detect if the surface characterizing device system is not operated correctly so that unsuitable measurements are likely to occur. The output of the pressure sensor may for example be used for ATR-based spectroscopy to indicate if sufficient pressure against the surface comprising a cured coat is established and maintained throughout the measurement. The output of the accelerometer may for example be used for both contact and non-contact based spectroscopy to indicate if the surface characterizing device system is held sufficiently steady for measuring.

In an embodiment said surface characterizing device system comprises pressure maintenance means, e.g. magnetic or suction means.

When the surface characterizing device system is configured to maintain a pressure itself, for example to apply magnetic force or suction force, it may advantageously ensure or at least facilitate sufficient pressure and stability during the measurement.

In an embodiment, said surface characterizing device system, such as a measurement device thereof, comprises a magnetic switchable device configured to releasable maintain attachment to said surface.

Magnetic switchable devices, also referred to as mechanically switchable magnets, may comprise a system of two or more permanent magnets arranged so that they can be rotated or displaced relative to each other, for example by rotating a rotatable magnet relative to a fixed magnet. By changing the relative configuration of two or more permanent magnets, the magnetic flux can be controlled through different paths, of which one path may provide an externally directed magnetic flux for attachment to external surfaces, and another path may keep the magnetic flux internally, thereby substantially switching the magnetic attachment off. In one example, the magnetic attachment is switched on when two magnets are aligned side by side with their north poles pointing in the same direction, and switched off when one of the magnets is rotated so that its south pole points in the same direction as the north pole of the other magnet, and vice versa. Levers, knobs or other handles may advantageously be provided for the user to operate the magnetic switchable device. Using a magnetic switchable device for magnetic attachment of the portable, battery-powered measurement device is advantageous because it is a purely mechanical, manual system, consuming no electric power from the battery and is easily operated by a user even for strong attachment configurations.

In an embodiment said surface characterizing device system comprises a spacer arranged to maintain a predefined distance to said surface comprising a cured coat during said spectroscopy measurement.

This may be advantageous for hyperspectral imaging camera-based spectroscopy, and may both facilitate keeping a suitable distance as well as holding the device sufficiently steady.

In an embodiment said sensor comprises an ultrasound transmitter and an ultrasound receiver.

In an embodiment said sensor comprises a camera sensor, such as a visible spectrum camera, and optionally a flash light.

In an embodiment said surface characterizing device system comprises one or more of an infrared flash light, a light sensor, a temperature sensor, a humidity sensor, a geolocation means such as GPS, a user interface, a visual output device.

In an embodiment the surface characterizing device system is handheld, and preferably at least the measurement device comprises handles.

In an embodiment the surface characterizing device system or at least the measurement device is transported by a robot or drone.

In an embodiment said surface characterizing device system is configured to perform a filtering or pre-validation of measurements and determine whether to temporarily store said representation of said measurement in said measurement buffer based on a result of said filtering or pre-validation.

In an embodiment said surface characterizing device system is configured to discard measurements that are not suitable for training or classification.

The ability to decide to not store measurement representations or discard measurements may be advantageous in order to not fill the measurement buffer with useless or non-optimal measurements and risk not being able to store relevant measurements. Rules or thresholds for determining when measurements are suitable may preferably be predefined, and for example relate to the minimum, maximum or average values in a measurement, to the color variation in an image measurement or other signal or statistical analysis thresholds, to input from supplementary sensors such as light sensor, accelerometer sensor, pressure sensor, etc. As a simple example, a spectroscopy measurement may be discarded if it does not contain values above a noise floor, or an image measurement may be discarded if the color variation is very small, e.g. all black pixels.

In an embodiment said surface characterizing device system is configured to indicate by said visual output device a result of said filtering or pre-validation or whether a measurement is suitable.

In an embodiment said surface characterizing device system comprises a trained classification model for characterizing a surface comprising a cured coat, and wherein said surface characterizing device system is configured to perform a classification based on said representation of said measurement and determine whether to temporarily store said representation on the basis of a result of said classification.

In an embodiment said trained classification model is configured to classify said surface into at least one predefined surface characteristic class from a plurality of predefined surface characteristic classes based on said measurement, the surface characteristic classes comprising one or more classes selected from a group of binder system classes, a group of coat manufacturer classes, a group of coating product classes, or a combination thereof.

In an embodiment said surface characterizing device system is configured to download an updated trained classification model upon reestablishment of said communication channel, optionally according to a time schedule.

When the surface characterizing device system is configured to perform local classification, it may be advantageous to download an updated version of the trained classification model from time to time. Typically it is not relevant to download a new version upon each reconnection, but for example once every 1 week, 2 weeks, 1 month, or 3 months.

In an embodiment said surface characterizing device system is configured to transmit a classification output from a trained classification model stored in said surface characterizing device system, preferably together with a classification model version number, and wherein said classification output is temporarily stored in said measurement buffer when the communication module is prevented from establishing said communication channel.

In an embodiment the surface is a surface of a coated structure preferably selected from the list of a ship hull, a tank interior, e.g. the inside of a ballast tank, a wind turbine tower, a wind turbine blade, a bridge, an oil rig, a building, a chimney or an industrial facility.

Using the classification method for identifying for example the coat binder on the surface of a ship hull or other exposed assets that may be critical, expensive or challenging to perform maintenance on, may be highly advantageous in order to confirm compatibility before overcoating with a fresh coating system. Classifying a contaminant, a contamination state or a degradation state for these kinds of assets may be highly advantageous for example to decide whether overcoating is due.

In an embodiment the surface is a surface of a coated structure comprising abase structure preferably of metal, such as steel, iron or aluminium, concrete, composites, such as reinforced composites e.g. glass fibre.

In an embodiment the method comprises cleaning said surface comprising a cured coat before establishing said measurement.

The cured coat may e.g. comprise one or more of the following binder systems: Acrylic, epoxy (including for example novolac epoxy and non-novolac epoxy), polyaspartic, polyurethane, polysiloxane, alkyd, silicate, silicone, polyurea, rosin, vinyl copolymers, polydimethylsiloxane, and hybrid technologies like for example epoxy/acrylic, epoxy/siloxane, epoxy/silicates. In the context of the invention, particular mention is made of epoxy, polyurethane and alkyd binder systems. Such itemizing of a number of possible binder systems may be an example of a plurality of predefined surface characteristic classes.

Classification of the manufacturer of a cured coat, or even a specific coating product, may be possible when different manufacturers and products use different specific binder configurations, different combinations of binder system, filler and/or pigment, different amounts of the various characteristic constituents, etc. Training based on spectroscopy measurements on training surfaces comprising cured coat from different manufacturers, or different coating products, together with labelling of the measurements in accordance with the known manufacturer or product name or number, may provide a trained classification model that can classify the manufacturer of a coating system or the specific coating product. When the coating product is identified, the properties, compatibilities and in some cases even the ingredients, such as binder system, may be possible to lookup from data sheets, thereby using the product identification to obtain further technical information about the surface that has been measured. The classification of coat products may be arranged at a level of detail according to the needs, such as classifying into a category of product types, more detailed into product families, series or even specific product numbers. A category of product type or product family may e.g. be defined according to product properties or features, field of application or overcoating compatibility.

The coating system may comprise several layers of cured coat, e.g. including a primer, e.g. an anticorrosive primer applied to the base surface. On top of one or more layers of primer, the coating system may include one or more layers of tie-coat or intermediate coat, underneath one or more layers of a top coat. The top coat may serve various purposes and may for example comprise an anticorrosive topcoat or a fouling control coat, such as a fouling control coat useful for marine structures. The topcoat may also be a decorative topcoat.

The individual layers, or single layer, of the cured coat may typically have a dry film thickness of 10-1000 μm, such as 10-600 μm, such as 20-500 μm, e.g. 75-400 μm. The spectroscopy measurements performed by the surface characterising device may typically extend to a depth of 1.0-2.5 μm from the outside of the surface into the cured coat. In typical embodiments the measured surface characteristics thereby solely relates to the outermost layer of the cured coat, typically the top coat.

In an embodiment said surface characterizing device system (SCD) is configured for use in electromagnetic shielding or weakening enclosures, for example steel enclosures such as tanks or cargo holds; concrete structures such as buildings, silos or basements.

The shielding effect of the enclosure does not have to be complete, nor does the enclosure have to be closed, for the invention to be beneficial. The communication module may be prevented from establishing a suitable communication channel even in cargo holds with open loading doors, underground parking facilities, indoors in buildings or steel structures such as ships, etc., whereby the present invention may become particularly advantageous in allowing to proceed with making measurements without a risk of losing them.

The invention relates in an aspect to a method of measuring a characteristic of a surface comprising a cured coat; the method comprising steps of: providing a surface characterizing device system in a first environment where communication with a cloud external to said first environment is prevented; using the surface characterizing device system to acquire a measurement of at least one characteristic of a surface comprising a cured coat; storing a representation of said measurement temporarily in a measurement buffer of said surface characterizing device system; providing said surface characterizing device system in a second environment; establishing a communication channel between said surface characterizing device system and said cloud; and transmitting said temporarily stored representation of said measurement to said cloud.

In an embodiment said method is implemented by a surface characterizing device system as described herein.

The invention relates in an aspect to a system comprising the surface characterizing device system as described herein and an unmanned vehicle such as a drone or robot, wherein the surface characterizing device system, or at least a measurement device, is mounted on or integrated in the unmanned vehicle.

The invention relates in an aspect to a computer-implemented training method for training a classification model for characterizing a surface comprising a cured coat; the training method comprising steps of receiving training input measurements from a surface characterizing device system as described herein based on measurements of training surfaces comprising a cured coat; generating labelled training input measurements by individually labelling said training input measurements in accordance with a plurality of predefined surface characteristic classes; establishing a training data set on the basis of said labelled training input measurements; providing a classification model; training said classification model based on said training data set to provide a trained classification model.

The invention relates in an aspect to a computer-implemented classification method of characterizing a surface comprising a cured coat, the method comprising steps of: providing a trained classification model as described herein; receiving an input measurement from a surface characterizing device system as described herein based on a measurement of said surface comprising a cured coat; and classifying said surface into at least one of said predefined surface characteristic classes based on said input measurement using said trained classification model to produce a classification output.

By enabling automatic characterization of a surface comprising a cured coat based on a number of predefined surface characteristic classes and trained on labelled measurements, for example labelled spectroscopy measurements, the usual requirement of relying on information recorded in logs or shipping a physical sample to a lab may be disregarded and instead surfaces can be characterized directly in the field, considerably faster and more convenient than before. The invention provides for various advantageous uses such as classification of a binder system of the cured coat. With additional information associated with the training input measurements such as manufacturer or product ID, for example in form of the labelling of training input measurements, it may advantageously also be possible to classify for example a manufacturer of the coating system or a specific coating system product to the extent the coating systems from different manufacturers are not identical. In addition to classifying a binder system, manufacturer and/or product, the training may allow classification of further useful surface characteristics such as a filler or pigment of the cured coat, a contaminant present in the surface together with the cured coat, a contamination state of the surface, a degradation type of the cured coat, a degradation state of the cured coat, etc.

Such classification results may be highly advantageous in determining for example strengths and vulnerabilities of an existing cured coat, the health and environmental impacts during use and disposal, the options for recycling, etc. Information about the binder type is furthermore important when application of new coat over an existing coat becomes relevant in order to select a suitable coating composition for overcoating to ensure compatibility with the existing coat.

The labelling of the training input measurements and use of the labelled training input measurements in the training of the classification model makes the training a supervised learning algorithm. While it can be challenging to provide labelled training data, supervised learning may still be highly advantageous in the context of characterizing a surface comprising a cured coat for the purposes of identifying the coating system or at least its binder system, or identifying contaminants in the coat.

Compared to machine learning in fields with abundance of data and easy access, like user behaviour on the Internet, image or speech recognition from online sources of images, audio and video, weather data analysis, stock market activity, health data based on wearable trackers etc., it is considerably more difficult to acquire large training data sets of in-field spectroscopy measurements of surfaces comprising cured coats. Although faster and much more convenient than shipping samples to a lab, it still requires specialized measuring equipment, trained operators and access to the relevant surfaces, to acquire each single spectroscopy measurement to use as training input measurement. By labelling the training data with the correct classes before training the classification model, it has turned out that acceptable classification results can be achieved from as little as about 20 training input measurements for each desired surface characteristic class to be recognized by the trained classification model. This is considerably less and highly advantageous over the usually advised amounts for unsupervised learning which are in the thousands or millions of training data examples. Further, due to the resources required to acquire spectroscopy measurements of surfaces comprising cured coats, compared to for example acquiring thousands or millions of data in seconds from the Internet in other fields, the resources required for labelling each spectroscopy measurement with the correct classes becomes comparable and acceptable for the smaller amounts of training input measurements required for the present invention.

Using labelled training input measurements for training the classification model may further have one or more of the advantages that the training algorithm achieves acceptable results much faster than with an unsupervised model, the classification model can be used right after training without post-training analysis of established classes, data for testing the classification model can be provided just by reserving some of the labelled input training data for testing (splitting), use of data augmentation, while possibly not that important in this context due to the small requirement for data amount, produces training data that are also labelled, etc. With unsupervised learning, the system would identify patterns, cluster data and create corresponding classes itself, but not necessarily classes suitable for the kind of coat identification or surface characterization that was intended. For example, an unsupervised learning model might end up classifying surfaces according to age or colour or filler or sea water content or unusable combinations of parameters, when it was actually information about the binder-system that was intended. Such challenges may be overcome in various embodiments of the present invention.

The classification method provides for automatic characterization in the field of a surface comprising a cured coat, for example with respect to identifying a binder-system or determining a manufacturer or product name of the coat.

The term “training surface comprising a cured coat” is a surface comprising a cured coat according to the above, for which a measurement, for example a spectroscopy measurement, may be used for training and/or testing a classification model. Some training surfaces comprising a cured coat may be laboratory samples, but preferably at least a part of the training surfaces comprising a cured coat are surfaces of in-field coated structures, e.g. a building or a ship, as described above. Likewise, the derived “training input measurements” simply refer to representations of measurements such as spectroscopy measurements made on training surfaces comprising a cured coat.

An identification or selection of a number of possible characteristics may be considered a plurality of predefined surface characteristic classes, such as exemplified above with the listing of e.g. specific binder systems together becoming a plurality of predefined binder classes. Thereby each selected, e.g., binder system becomes classifiable by the invention after training.

Surface characteristic classes of the same or similar type or category may be referred to as a class group, for example is binder classes considered a class group, and for example is coat product classes considered another class group. For various purposes it may be advantageous to implement the training classification with focus on only one class group of predefined surface characteristic classes, for example only binder classes or only coat product classes, etc. Thereby both the training and classification can be made simpler and require less resources.

For various purposes it may be advantageous to implement training and classification of several of the mentioned class groups, for example both binder classes and coat product classes, etc. Thereby the classification model can be used for versatile purposes and/or classify several properties of the surface comprising a cured coat in one go.

Classification of a binder system, among for example epoxy, polyurethane and alkyd, may be advantageous in order to ensure application of a compatible coating system during maintenance or repair, to determine properties, e.g. strengths and vulnerabilities, of an existing cured coat, to determine the health and environmental impacts during use and disposal, and/or to determine the options for recycling, etc.

The cured coat being measured may in practice comprise a composition of binder systems, fillers, pigments, and other components. Further, the surface comprising a cured coat of some age in the field may comprise further compounds besides the cured coat, such as water absorption, and/or comprise chemical changes in the binder system etc. due to partial degradation of the cured coat. The spectroscopy measurements therefore are influenced by and comprise information about all the compounds in the surface composition which interact with the selected electromagnetic radiation spectrum being measured, and the absorbance spectrum may become quite complex compared to spectroscopy measurements on single compounds or simpler compositions. Human or automatic rule-based compound identification based on identification of a few characteristic absorbance peaks is therefore made difficult or even practically impossible. Embodiments of the present invention may advantageously provide a way to assess such complex and often similar-looking spectroscopy measurements to classify one or more ingredients, binders, fillers and pigments, water absorption, etc.

Training based on spectroscopy measurements in the field of the complex compositions from real-life assets that the surface comprising a coat constitutes, may advantageously produce a robust trained classification model that reliably may categorize complex cured coat compositions and contamination and degradation. Further, such training may also teach the classification model to distinguish between, for example, same binders from different products or different manufacturers due to combination with different use of fillers, pigments, etc. A corpus of measurements to use as training data may preferably encompass as much variation as possible within the desired surface characteristic classes, in order to establish a robust and reliable trained classification model. Variation may for example be achieved by measuring different surfaces with the same coating system, different product variations (e.g. filler and pigment combinations) with the same binder system, variation in age of coating, variation in environmental exposure of coating, variation in mechanical damages of surface and coating, etc. In a particular embodiment, surfaces are measured as soon as freshly applied coating systems have cured and the measurement stored as a baseline ID profile of this coating system. Later measurements of the same surfaces provide ID profiles where similarities to the baseline ID profile may be indicative of the coating system, and differences to the baseline ID profile may be indicative of degradation and other changes caused by aging, damage, environmental exposure, etc.

The individual labelling of training input measurements in accordance with the plurality of predefined surface characteristic classes refers to assigning each training input measurement to one or more surface characteristic classes. Simply put, the labelling assigns a correct class to each measurement, allowing the training system to manipulate parameters of the classification model towards recognizing these correctly when later receiving an unlabelled measurement. When for example binder systems are considered, each training input measurement should normally be labelled with one binder class from the plurality of predefined surface characteristic classes in accordance with the binder system of the cured coat of the corresponding training surface. In some embodiments two or more binder classes may be assigned to one training input measurement, for example in a mixed composition. When several groups of characteristics, e.g. both manufacturers and binder systems, are considered, the training input measurements should normally be labelled with one or more predefined surface characteristic for each group. In some embodiments, when a correct allocation cannot be made for all groups of characteristics, e.g. both binder and manufacturer, only the more certain labelling should be assigned. The assignment of predefined surface characteristic classes to the training input measurements, i.e. the labelling step, may for example be performed in connection with performing the spectroscopy measurements, at a pre-processing or data preparation step, or anywhere later in the process before initiating the training. The training input label, i.e. the designated surface characteristic class for the measurement, may be incorporated into the measurement data package to form the labelled training input measurement, be transmitted or handled together with the measurement data package as a separate piece of information whereby the measurement and label together form the labelled training input measurement, or be transmitted or handled separately from each other with a link or pointer from the measurement to the label or predefined surface characteristic class, whereby the measurement and the link or pointer form the labelled training input data.

As used herein, “classification” refers to categorizing data into classes. More particularly in the present context, to categorize input measurements, and thereby the surfaces subject to the input measurements, into predefined surface characteristic classes. A classification output thereby provides an indication of which characteristic, if any, among the predefined characteristics, the surface possesses.

“Classification model” is understood as a practical implementation of a classification concept or principle suitable to categorizing data. Several such classification concepts and principles with various advantages and disadvantages are available to the skilled person to choose from, for example neural networks, decision trees, support vector machines, Bayesian statistics modelling, logistic regression, etc. In the present context of spectroscopy measurements, the inventors have identified neural networks and sub-algorithms such as convolutional neural networks as examples of suitable classification concepts, and examples of practical implementations in the form of classification models will be described below, for example a so-called VGG16 convolutional neural network. Several other classification concepts will also be suitable for the present purpose of surface characterization based on spectroscopy data.

In the present context, “training method” and “training” refers in general to preparing training data and using them to modify the configuration and parameters of the classification model until it achieves a desired reliability in classifying data, and/or until it does not improve further with the available amount of training data and predefined constraints on the classification concept and configuration. The output of the training method and the training of the classification model is therefore referred to as a trained classification model, which may then be used to classify other spectroscopy measurements into the same predefined surface characteristic classes. Data preparation may advantageously comprise steps of pre-processing to e.g. normalize, noise-reduce, compact, etc., the measurements, and data augmentation to provide variants and synthesize a larger diversity to increase robustness, etc. Modification of classification model parameters using the training data may be performed in different ways depending on the selected classification concept and configuration. Training may involve performing a classification operation on one or more training input measurements, compare the resulting outputs with the labels representing the correct outputs, using the result of the comparison to modify parameters of the classification model, e.g. weights and coefficients, and repeat the training process with same or different training input measurements to further improve, until a stop criterium is reached. The prepared data ready for use for training are referred to as a “training data set”. The classification model with its uniquely modified parameters after the training stop criterium is reached, is referred to as a “trained classification model”, which can now be used to classify unknown surfaces comprising a cured coat.

The invention relates in an aspect to a training system configured to train a classification model for characterizing a surface comprising a cured coat; the training system comprising: a training input measurement receiver configured to receive training input measurements from a surface characterizing device system as described herein based on measurements of training surfaces comprising a cured coat; a training input measurement labeller configured to generate labelled training input measurements by individually labelling said training input measurements in accordance with a plurality of predefined surface characteristic classes; a training data set generator configured to generate a training data set on the basis of said labelled training input measurements; a classification model; a training module configured to train said classification model based on said training data set to provide a trained classification model.

The invention relates in an aspect to a trained classification model for characterizing a surface comprising a cured coat; the trained classification model being established by the training method or the training system described herein.

The invention relates in an aspect to a classification system configured to characterize a surface comprising a cured coat, the classification system comprising: a trained classification model as described herein or provided according to the training method described herein or provided by the training system described herein; an input measurement receiver configured receive an input measurement from a surface characterizing device system as described herein based on a measurement of said surface comprising a cured coat; a classifier configured to classify said surface into at least one of said predefined surface characteristic classes based on said input measurement using said trained classification model to produce a classification output.

The invention relates in an aspect to a computer-implemented classification method of characterizing a surface comprising a cured coat, the method comprising steps of: receiving an input measurement from a surface characterizing device system as described herein based on a measurement of said surface comprising a cured coat; an classifying said surface into at least one of a plurality of predefined surface characteristic classes based on said input measurement using a trained classification model as described herein or provided according to the training method described herein or provided by the training system described herein to produce a classification output.

The invention relates in an aspect to a use of the classification method described herein, or the classification system described herein, to identify, for a surface comprising a cured coat, one or more of a coat binder system, a coat filler or pigment, a contaminant, a coat manufacturer, a coat product, a contamination state, a degradation state or to determine whether a coating system is compatible with said surface comprising a cured coat, or to determine whether overcoating of said surface comprising a cured coat is due.

The invention relates in an aspect to a training data set for use in the training method or the training system described herein, comprising labelled training input measurements based on measurements of training surfaces and labelled in accordance with a plurality of predefined surface characteristic classes.

In an embodiment the training data set comprises at least 10, such as at least 20, preferably at least 40, labelled training input measurements for each of said predefined surface characteristic classes.

In an embodiment each of said labelled training input measurements comprises an array of values based on absorbance or reflectance measured at different wavelengths on one of said training surfaces and an associated training input label designating one or of said predefined surface characteristic classes.

In an embodiment each of said labelled training input measurements comprises an image generated from absorbance or reflectance measurements at different wavelengths on one of said training surfaces and an associated training input label designating one or of said predefined surface characteristic classes.

In an embodiment said plurality of predefined surface characteristic classes comprises one or more classes selected from a group of binder system classes, a group of coat manufacturer classes, a group of coating product classes, or a combination thereof.

In an embodiment said plurality of predefined surface characteristic classes is a plurality of classes selected from the group of binder system classes.

In an embodiment said group of binder system classes comprises one or more class from the list of acrylic, epoxy (including for example novolac epoxy and non-novolac epoxy), polyaspartic, polyurethane, polysiloxane, alkyd, silicate, silicone, polyurea, rosin, vinyl copolymers, poly dimethylsiloxane, and hybrid technologies like for example epoxy/acrylic, epoxy/siloxane and epoxy/silicates binder classes, preferably at least one or more of the classes of epoxy, polyurethane and alkyd binder system classes. In an embodiment, the binder system classification is the only classification carried out, thereby providing a simpler model and system to analyse a very important and useful surface characteristic.

The label for a binder system class need not necessarily include the name of a binder system, but can also refer to a grouping or category of coats that share similar binder systems, for example referring to a certain property of a group of binder systems. Further, binder systems based on the same binder may be grouped and classified under a common label, such as assigning a single “epoxy”-class to all coats comprising epoxy binders. In circumstances where the difference between variants of the binder system, e.g. novolac epoxy and non-novolac epoxy, is considered sufficiently significant, different labels can be used for these two, e.g. epoxy-based, binder systems to be able to classify them individually. For even higher classification resolution, where relevant, the labelling can further be split up in specific binder formulations, e.g. bisphenol-A based epoxy resin, bisphenol B based epoxy resin, etc. Any of the above-described levels of grouping can be considered binder classes.

In an embodiment said predefined surface characteristic classes comprises a class for unknown outcomes and/or unsuitable input measurements.

It may be advantageous to assign a class to the situation where the output of a classification does not fulfil a predetermined criteria, e.g. a probability threshold, for any of the other classes. Such a class may be called “unknown” or similar. It may be advantageous to monitor how often the “unknown”-class becomes the classification output, as a frequent use thereof may indicate either an inaccurate trained classification model or a demand to add an additional known class, e.g. for a new binder type that is becoming popular. It may be advantageous to assign a class to use for input measurements that are not suitable for classification, for example due to measuring operation error, measuring device error, etc. The classification into the “unsuitable”-class may be based on the trained classification model being training to identify such unsuitable input measurements, or it may rely on for example a rule-based evaluation of input measurements, e.g. the measurement comprising all zeroes, overruling the classification to assign the “unsuitable”-class and disqualify any other class the classifier may have identified.

In an embodiment said predefined surface characteristic classes do not comprise pigment classes or color classes.

In an embodiment each of said input measurements or labelled training input measurements is based on spectroscopy measurement of at least 20 different wavelengths, such as least 50 different wavelengths, preferably at least 100 different wavelengths, for example at least 150 different wavelengths, such as at least 250 different wavelengths.

In an embodiment the at least 20 different wavelengths are in the infrared IR range, such as selected from one or more of: the mid infrared MIR range, for example the spectroscopic fingerprint region, the functional group region, or a combination thereof; the near infrared NIR range, the short wavelength infrared SWIR range, or a combination thereof; the medium wavelength infrared MWIR range, the long wavelength infrared LWIR range, or a combination thereof; the range of 8,000 nm to 10,500 nm, the range of 5,500 nm to 8,000 nm, the range of 2,500 nm to 20,000 nm, the range of 4,000 nm to 12,000 nm, the range of 5,500 nm to 10,500 nm, or a combination thereof.

The selection of an IR wavelength range may be determined in consideration of the classification purpose and the technology options. Regarding classification purpose, different wavelength ranges may be more or less suitable for classifying different properties of the surface. For example may one wavelength range be dominated by characteristics of binders, while another wavelength range may be dominated by characteristics of pigments and fillers; or one wavelength range may be influenced less by irrelevant properties which in another wavelength range may overshadow the relevant properties. When the classification purpose is to characterize a binder of a coating, a wavelength range where binder-characteristics are prevalent and/or a wavelength range where non-binder characteristics are reduced, may be selected. When the purpose of an embodiment is to classify a coating product by combination of binder, filler and pigment, selecting a different wavelength range may be more efficient, and yet another wavelength range may be optimal for classifying contamination types, etc. The range of 5,500 nm to 8,000 nm may be particularly appropriate for artificial intelligence-assisted IR identification of coating binder types because many peaks originating from the chemical bonds of organic binders exist in this range, and in addition the range is generally less influenced by inorganic filler and pigment components of the coatings. With regards to binder peaks the range of 5,500 nm to 8,000 nm may for example contain signals from C—O, C═O, C—N, C—C (aromatic), etc. These chemical bonds are generally found in the reactive curing groups and the backbone structure of the binder resins of industrial coatings. The different amounts of signals in each binder type enables the assisted analysis of this wavelength range for determining the binder type. As another example, the wavelength range of 8,000 nm to 10,500 nm, while also capturing binder-relevant peaks from for example the epoxide of an epoxy binder, in the same region captures large IR peaks from inorganic filler/pigment components that may overshadow the binder peaks due to their large content relative to binder, making this wavelength region better suited for classification purposes that also depend on fillers/pigments. The ranges of 2,500 nm to 20,000 nm, or 4,000 nm to 12,000 nm, or 5,500 nm to 10,500 nm, for example, are broad ranges covering a large part of both the functional group region and the fingerprint region, thereby generally applicable to many classification purposes, but may require combination of several different sensors to cover the broad ranges. From an implementation perspective, when hyperspectral imaging technologies are preferred, the NIR and SWIR ranges, for example the ranges from 750 nm to 2,500 nm, or 970 nm to 2,500 nm, may be better compatible with the available technologies.

In an embodiment the spectroscopy measurements are obtained by attenuated total reflectance ATR spectroscopy, preferably single-bounce ATR, preferably using a tunable pyroelectric detector to obtain measurements at different wavelengths.

In an embodiment the spectroscopy measurements are obtained by multispectral imaging or hyperspectral imaging.

In an embodiment the spectroscopy measurements are obtained within a distance of less than 10 μm, such as less than 5 μm, such as less than 4 or 3 or 2 or 1 μm, for example less than 0.5 or 0.1 μm, preferably during contact in at least one contact spot, more preferably a plurality of contact spots, with the surface comprising a cured coat.

With contact-based spectroscopy such as attenuated total reflection ATR techniques the spectroscopy measurements should preferably be performed while the measuring point of the spectroscopy hardware, e.g. implemented in a surface characterizing device system, is in contact with the surface comprising a cured coat. However, surface roughness, which applies to all relevant surfaces in practice, cause contact to occur at discrete contact spots. The number and/or area of discrete contact spots may be increased by applying pressure to the measuring point against the surface, and/or by reducing surface roughness. When implementing ATR-based spectroscopy, the measurement point of the measuring device should, at the time of taking the measurement, be in such proximity or contact that evanescent waves may extend from the ATR crystal into the surface comprising a cured coat and be reflected back into the ATR crystal with a measurable intensity and angle. The specific contact requirements for achieving a sufficiently measurable reflection depends on the contact area and properties of the ATR crystal, the roughness, hardness and other properties of the surface comprising a cured coat, the wavelength range being measured, the beam intensity and collimation or focus, etc., and is derivable by a skilled person within the field of ATR spectroscopy.

In an embodiment the spectroscopy measurements are obtained while maintaining a pressure of a spectroscopy measurement device against said surface of at least 5 kg, such as at least 10 kg, preferably at least 20 kg, such as at least about 10,000 kPa, such as at least 25,000 kPa, preferably at least about 50,000 kPa, for example at least 100 kgf/cm², such as at least 250 kgf/cm² or at least 500 kgf/cm².

Performing the spectroscopy measurements directly on the surface, and preferably applying a pressure of the, for example, ATR crystal against the surface, may be advantageous in keeping other disturbances, e.g. emission and reflection from other surfaces and objects, out and allow the reflectance or absorbance to depend solely on a small area of the surface being measured. Even a small amount of pressure, achievable by most operators, may be sufficient to improve the results. As mentioned above, the applied pressure may among others serve to increase the number and/or area of discrete contact spots between the measurement point, e.g. ATR crystal, and the surface comprising a cured coat. A pressure sensor may be provided in the measurement device to inform the user of the applied pressure, to perform regulation of pressure applied by a drone, robot or other automatic device operator, and/or to validate measurements in light of a minimum pressure criterium or acceptable pressure range. The measurement device, e.g. a surface characterizing device system, may comprise means for applying or maintaining a certain pressure itself, for example by magnetic force, suitable for e.g. steel structures, or suction force, suitable for most flat surfaces. A pressure-sensor as described may advantageously be employed to regulate the self-applied or self-maintained pressure.

In an embodiment the pressure of a spectroscopy measurement device against said surface is maintained by means of a magnetic switchable device. Such a device has been described above.

In an embodiment the spectroscopy measurements are obtained at a distance of at least 10 μm, such as at least 1 mm, 5 mm, 1 cm, 2.5 cm or 5 cm, for example at least 50 cm or 1 meter, from the surface comprising a cured coat.

Performing the spectroscopy measurements of the surface from a distance makes it possible to utilize non-contact or remote spectroscopy technologies such as hyperspectral imaging cameras. In various embodiments such techniques may be designed for small distances where a lens of for example a hyperspectral imaging camera is maintained at a predefined or variable distance from the surface comprising a cured coat by means of a spacer, e.g. a distance of 0.1 to 10 cm from the surface comprising a cured coat, or may be designed for larger distances where the hyperspectral imaging camera may be positioned in a most convenient way at a distance of, for example, 0.05 to 10 m from the surface comprising a cured coat.

In an embodiment said classification model is a supervised classification model.

In an embodiment said classification model comprises a nonlinear model.

In an embodiment said classification model comprises at least two different types of activation functions.

In an embodiment said classification model comprises at least a SoftMax activation function.

In an embodiment said classification model comprises at least a ReLu type activation function.

In an embodiment said classification model is a neural network.

In an embodiment said classification model is a convolutional neural network.

In an embodiment said classification model comprises a feature extraction module.

Advantageously, the feature extraction module may during training of the classification module learn features that may provide better classification of surface characteristic classes, when the classification model has been trained and is applied for classification.

In an embodiment said classification model comprises: a feature extraction module configured to receive training input measurements and to generate a feature extraction output based on said training input measurements; and a classification module configured to classify based on the feature extraction output of the feature extraction module.

Advantageously, this has the effect that during training, the classification model may automatically learn features of the training input measurement that are useful for distinguishing different surface characteristic classes. Thereby, the performance of the classification module may improve.

In an embodiment training of said classification model comprises optimizing a cost function, and wherein said cost function is based on categorical cross entropy.

In an embodiment parameters of the classification model may be optimized based on grid and/or random search for optimal parameters and/or based on cross validation.

In an embodiment said classification model comprises one or more from the list comprising: a gradient boosting model, a decision tree, a support vector machine, a neural network, a Bayesian based model.

In an embodiment said classification model comprises an XGboost model.

In an embodiment said classification model is pretrained based on transfer learning.

In an embodiment a size of the training input measurements and/or said input measurements is reduced based on dimensionality reduction and/or based on feature selection.

In an embodiment the dimensionality reduction and/or feature selection is based on one or more from the list comprising: principle component analysis, elastic net.

Advantageously this has the effect that the size of the training input measurements is reduced, and thereby reducing computer resource demands and/or reducing data processing time.

Advantageously, utilizing elastic net may handle regularization and recue the weight of potential useless features that do not provide any benefit with regards to the performance of the trained classification model with regards to classifying surface characteristic classes.

Advantageously, utilizing principle component analysis may group features based on variance and thereby reduce the size of the training input measurements without substantially compromising performance of the trained classification model. Such data reduction may advantageously shorten the time required to train the classification model, and the time required for classifying surface characteristic classes based on training input measurements.

In an embodiment said training input measurements and/or said input measurements are smoothed.

In an embodiment said training input measurements and/or said input measurements are smoothed using a Savitzky-Golay filter.

In an embodiment said step of training comprises steps of establishing a test data set on the basis of said labelled training input measurements; generating test classification outputs using said test data set and said trained classification model; evaluating said test classification outputs on the basis of test success criteria to determine a trained classification model quality; selecting on the basis of said trained classification model quality to repeat or not repeat said step of training.

In an embodiment the trained classification model is updated by re-training or transfer learning according to one or more update trigger from the list of: receipt of new training input measurements, such as when at least 10, 50, 100, 200 or 1000 new training input measurements are received, expiry of an update deadline, such as after 1 week, after 2 weeks, after 1 month, after 3 months, after 6 months, or after 1 year, establishment of a new surface characteristic class or modification of an existing surface characteristic class.

In an embodiment metadata relating to said surface, such as asset type, asset identification or most recently applied coating system, is assigned to said representation of said measurement.

In an embodiment metadata relating to geolocation, weather, temperature, humidity, light or operator, is assigned to said representation of said measurement. Metadata may also comprise information about periods where said communication module has been prevented from establishing said communication channel, i.e. when the surface characterizing device system has been online and offline, respectively.

In an embodiment the step of training said classification model is performed by a cloud computing system.

In an embodiment the step of classifying said surface is performed by a cloud computing system.

In an embodiment said trained classification model is transferred from said cloud computing system to one or more of said surface characterizing device systems, such as or a smart device thereof, for performing said step of classifying a surface locally.

While training requires access to as many training data as possible, is relatively more resource demanding, and may benefit several surface characterizing device systems, and therefor is preferably performed in a central system, for example a cloud computing system, the classifying of a surface based on a trained classification model is less resource demanding and only requires a single measurement, may be carried out centrally to simplify resources at the site of the surface, or be carried out locally by the surface characterizing device system or a smart device to allow quick and local classification, even in lack of cloud connection.

In an embodiment said plurality of predefined surface characteristic classes further comprises one or more classes selected from a group of filler and pigment classes, a group of contaminant classes, a group of contamination state classes, a group of degradation type classes, a group of degradation state classes, or a combination thereof.

Thereby further surface characteristics may be identified, for example more information about the original coat, or information about the current condition of the coat and/or surface.

The one or more cured coats may comprise one or more pigments, for example pigments suitable for changing the aesthetic appearance by e.g. providing a colour and/or pigments with anticorrosive properties. Examples of such pigments are: zinc oxide, zinc containing phosphate and polyphosphate, aluminium containing phosphate, zinc borate, graphite, carbon black oxide, coated mica, fluorescent pigments, cuprous oxide, aluminium paste pigment (leafing and non-leafing type), metallic pigments, zinc dust, organic pearl pigment, ammonium polyphosphate, coloured silica sand, polyacrylic acid/calcium carbonate, azo-, phthalocyanine and anthraquinone derivatives (organic pigments), and titanium dioxide (titanium(IV) oxide), etc. Such itemizing of a number of possible pigments may be an example of a plurality of predefined surface characteristic classes.

The coating system may e.g. comprise one or more fillers selected from for example: Carbonates such as: Calcium carbonate, calcite, dolomite (=calcium/magnesium carbonate), magnesium silicate/carbonate, polycarbonate. Included are also mixtures, calcined grades and surface treated grades. Silicates such as: Aluminium silicate (kaolin, china clay), Magnesium silicate (talc, talc/chlorite), Potassium Aluminium silicate (plastorite, glimmer), Potassium Sodium Aluminium silicate (nepheline syenite), Calcium silicate (wollastonite), Aluminium silicate (bentonite), phyllo silicate (mica). Oxides: Silicon dioxide such as quartz, diatomite, metal oxides such as calcium oxide, aluminium oxide. Hydroxides/hydrates such as: Aluminium hydroxide, Aluminium trihydrate, Sulphates: barium sulphate. Other fillers: Barium metaborate, silicon carbide, Perlite (volcanic glass), Glass spheres (solid and hollow), glass flakes, glass and silicate fibres, organic fibres, polyvinylidene chloride acrylonitrile, polystyrene acrylate. Such itemizing of a number of possible fillers may be an example of a plurality of predefined surface characteristic classes.

Included are also mixtures of the above fillers as well as grades which are natural, synthetic, calcined or surface treated.

Depending on exposure to various environments and impacts, the surface comprising the cured coat may also comprise one or more contaminants, in addition to the cured coat. Depending on the type of contaminant and the degree or state of contamination, this may affect the properties and lifetime of the cured coat. It may therefore be advantageous to classify the contaminants present in the surface, such as water absorption, deposits of organic material, etc., and/or the state or degree thereof.

Exposure to various environments and/or simply time going by may also cause the coating system to degrade, also referred to as deteriorate. Chemical or physical degradation may for example be caused by harsh environments like salt water, acid, solvents, polluted air, heavy particle-carrying winds, etc., exposure to various biological or inorganic compounds, UV radiation from the sun, heat from the sun or the environment, etc. Depending on the type of degradation and the degree or state of degradation, this may affect the properties and lifetime of the cured coat. It may therefore be advantageous to classify the degradation or deterioration of the cured coat, such as being chemically decomposed or transformed.

It may be advantageous to classify the state of contamination and/or the state of degradation in addition to or instead of simply providing the contaminant or type of degradation. With the indication of a state of contamination or degradation, the ability to evaluate the technical situation and make a qualified advice or decision with respect to vulnerabilities of the coat, maintenance, or impact on environment or health, may be improved.

It may be advantageous to provide for classification into combinations of above classes or into several of the above classes. For example, the classification model may be trained to output both a binder class and a degradation class, or a binder class, filler and pigment class, or a product class, a degradation class and a contaminant class, or any other combination of classes. A classification system according to the invention may then receive a spectroscopy measurement of a surface comprising a cured coat, and output two or more classes of different types, such as identifying the binder as epoxy-based and at the same time provide a state of water absorption.

In another example, several classification models are trained for various class groups, for example one classification model trained to classify into only binder classes and another classification model trained to classify into only contaminant classes, etc. A classification system according to the invention may then receive a spectroscopy measurement of a surface comprising a cured coat, run the same input through two or more trained classification models, preferably in parallel, and output one or more class from each classification model.

In an embodiment said filler classes comprise one or more class from the list of carbonates such as: calcium carbonate, calcite, dolomite (=calcium/magnesium carbonate), magnesium silicate/carbonate, polycarbonate, calcined grades, surface treated grades, and mixtures thereof; silicates such as: Aluminium silicate (kaolin, china clay), Magnesium silicate (talc, talc/chlorite), Potassium Aluminium silicate (plastorite, glimmer), Potassium Sodium Aluminium silicate (nepheline syenite), Calcium silicate (wollastonite), Aluminium silicate (bentonite), phyllo silicate (mica); oxides such as: Silicon dioxide such as quartz, diatomite, and metal oxides such as calcium oxide, aluminium oxide; hydroxides/hydrates such as: Aluminium hydroxide, Aluminium trihydrate, Sulphates: barium sulphate; and other fillers such as: Barium metaborate, silicon carbide, Perlite (volcanic glass), glass spheres (solid and hollow), glass flakes, glass and silicate fibres, organic fibres, polyvinylidene chloride acrylonitrile and polystyrene acrylate.

In an embodiment said pigment classes comprise one or more class from the list of zinc oxide, zinc containing phosphate and polyphosphate, aluminium containing phosphate, zinc borate, graphite, carbon black oxide, coated mica, fluorescent pigments, cuprous oxide, aluminium paste pigment (leafing and non-leafing type), metallic pigments, zinc dust, organic pearl pigment, ammonium polyphosphate, coloured silica sand, polyacrylic acid/calcium carbonate, azo-, phthalocyanine and anthraquinone derivatives (organic pigments) and titanium dioxide (titanium(IV) oxide).

In an embodiment said contaminant classes comprise one or more class from the list of water absorption, contaminant absorption, corrosive substances and organic material absorption.

In an embodiment said degradation type classes comprise one or more class from the list of chemical change classes such as chemical decomposition or chemical oxidation; and physical change classes such as structural changes, phase changes.

The classification into a degradation type class may for example rely on an expected and intended chemical bond characterising the binder that is reduced or missing or a spectroscopic ID profile not being found sufficiently clearly, thereby indicating that this expected element has degraded into something else, or it may for example rely on the identification of a chemical bond or spectroscopic ID profile that is known to emerge from the intended coat constituents when the coat is degrading. In other words, the classification model may be trained to select degradation type classes when intended coat characteristics are absent, and/or when degradation characteristics are present.

In an embodiment the training method is for training a classification model for characterizing a surface comprising a cured coat in a surface characterization system comprising a fleet of decentral surface characterizing device systems and a central training system, and wherein the training method comprises steps of a first subset of said fleet of decentral surface characterizing device systems establishing training input measurements based on measurements of training surfaces comprising a cured coat; said training input measurements being transmitted to said central training system; a second subset of said fleet of decentral surface characterizing device systems establishing input measurements based on measurements of surfaces comprising a cured coat; and said central training system training a classification model based on said training input measurements received from said first subset of said fleet to provide a trained classification model.

In an embodiment an operating method of the surface characterization system comprising a fleet of decentral surface characterizing device systems, a central training system and a classification system, comprises steps of providing the trained classification model by the training method; and classifying surfaces comprising a cured coat based on input measurements acquired by surface characterizing device systems of said second subset of said fleet using said trained classification model to produce classification outputs, the input measurements being based on measurements of the surfaces comprising a cured coat

According to an embodiment, the first subset of surface characterizing device systems may thereby advantageously be commissioned to improve the utilization of the second subset of surface characterizing device systems to classify surfaces comprising a cured coat. The improvement may be caused by providing training input measurements usable by the central training system to improve the trained classification model, which is used for characterizing the surfaces.

A surface characterization device according to the invention may thereby be improved based on inputs from numerous other devices instead of having a fixed usability or only learning from its own measurements. Preferably, the first subset may comprise tens or hundreds of surface characterizing device systems delivering training data for the training system, thereby increasing the data corpus manifold compared to what is achievable by individual devices even over a long time. A large amount of training data also enables classifying more classes, classes closer to each other, utilization of more complex models such as deep learning, etc.

Training a classification model on training data received from different sources, and thereby inherently demonstrating larger variation than typically seen in data from a single source, may also be advantageous in achieving a robust and reliable trained classification model.

Operating the system so that only a specific subset of surface characterizing device systems contribute to the training may be highly advantageous, for example with respect to the reliability and availability of training data. Various rules may be set to designate surface characterizing device systems to the first, second or both subsets, for example based on the device's capabilities, the operator's experience, etc.

A “measurement” of a surface or training surface is preferably a measurement suitable to be performed by a portable, battery powered device. The “measurement” may consist of several different kinds of measurement or outputs of several sensors, possibly of different type, technology and aim. It may for example be advantageous to perform a spectroscopy measurement to identify ID profiles or individual constituents of the cured coat and possibly contaminants, such as for example attenuated total reflection ATR spectroscopy or hyperspectral imaging spectroscopy. It may also or in addition be advantageous to take a picture with a visible spectrum camera to evaluate surface characteristics such as surface roughness or defects, for example cracks, scratches, blisters, etc. The measurement may also include supplementary measurements to enhance or validate the results from the main sensors, or aid the operator in making suitable measurements.

The invention relates in an aspect to surface characterization system configured to characterize surfaces comprising a cured coat, the surface characterization system comprising a fleet of decentral surface characterizing device systems, said fleet comprising a first subset of said fleet of decentral surface characterizing device systems configured to establish training input measurements based on measurements of training surfaces comprising a cured coat; and a second subset of said fleet of decentral surface characterizing device systems configured to establish input measurements based on measurements of said surfaces comprising a cured coat; the surface characterization system comprising a central training system comprising a classification model and a training module configured to train said classification model based on said training input measurements from said first subset of said fleet to provide a trained classification model; and the surface characterization system comprising a classification system comprising a classifier configured to classify said surfaces based on said input measurements from said second subset of said fleet using said trained classification model to produce classification outputs.

In an embodiment the surface characterizing device systems are surface characterizing device systems as described herein.

In an embodiment the classification method of characterizing surfaces comprising a cured coat comprises steps of establishing by a central training system a trained classification model based on training input measurements, the training input measurements being based on measurements of training surfaces comprising a cured coat acquired by a first subset of a fleet of decentral surface characterizing device systems; acquiring by a second subset of said fleet of decentral surface characterizing device systems input measurements based on measurements of said surfaces comprising a cured coat; and classifying said surfaces based on said input measurements using said trained classification model to produce classification outputs.

The invention relates in an aspect to a fleet of decentral surface characterizing device systems, said fleet comprising a first subset and a second subset, wherein said first subset of said fleet of decentral surface characterizing device systems being configured to establish training input measurements based on measurements of training surfaces comprising a cured coat and transmit said training input measurements to a central training system; and said second subset of said fleet of decentral surface characterizing device systems being configured to establish input measurements based on measurements of surfaces comprising a cured coat and transmit said input measurements to a classification system comprising a classifier.

In an embodiment the surface characterizing device systems are surface characterizing device systems as described herein.

In an embodiment said training system trains said classification model based on said training input measurement from said first subset and not from said second subset of said fleet.

In an embodiment said training input measurements have associated training input labels in accordance with a plurality of predefined surface characteristic classes.

The training input labels may be received together with the training input measurements they are associated with, or the training input labels may be assigned to the training input measurements based on information established in the training system, or received from e.g. external systems, e.g. maintenance databases.

In an embodiment the method comprises generating labelled training input measurements by individually labelling said training input measurements in accordance with a plurality of predefined surface characteristic classes; and wherein said training said classification model based on said training input measurements comprises training said classification model on the basis of said labelled training input measurements.

In an embodiment the training input measurements with associated training input labels are transmitted in the form of labelled training input measurements.

In an embodiment the method comprises establishing a training data set on the basis of said training input measurements and associated training input labels.

In an embodiment said classifying comprises classifying said surface into at least one predefined surface characteristic class.

In an embodiment said classification outputs are provided to respective of said surface characterizing device systems of said second subset of said fleet.

In an embodiment said decentral surface characterizing device systems of said second subset comprise said classification system, and wherein said classification system is configured to classify said surfaces based on said input measurements using a trained classification model received from said central training system to produce classification outputs.

In an embodiment said fleet comprises two or more surface characterizing device systems, such as at least 5, 10, 20 or 100 surface characterizing device systems.

In an embodiment said first subset comprises two or more surface characterizing device systems, such as at least 5, 10, 20 or 100 surface characterizing device systems.

In an embodiment said second subset comprises one or more surface characterizing device systems, such as at least 2, 5, 10, 20 or 100 surface characterizing device systems.

In an embodiment said first subset and said second subset are different.

In an embodiment said subsets overlap by at least one, but preferably not all, such as for example 1, 2, 5 or 10, surface characterizing device systems providing both training input measurements and input measurements.

In an embodiment the surface characterizing device systems of both subsets of the subsets are identical or comprises identical sensors or are configured to perform measurements of the same type, such as spectroscopy, or single-bounce ATR spectroscopy.

In an embodiment the belonging of a surface characterizing device system to the first subset is evaluated for each measurement, for each surface, for each coated structure, for each change of operator or for each predefined time duration such as 1 day, 1 week, 2 weeks, 1 months, 3 months, 6 months or 1 year.

### THE DRAWINGS

Various embodiments of the invention will in the following be described with reference to the drawings where applicable.

FIG. 1 illustrates examples of coated structures of use cases for the invention,

FIGS. 2-6 illustrate coated structures and surfaces comprising a cured coat according to the invention,

FIGS. 7-8 illustrate aspects of obtaining measurements in various embodiments of the invention,

FIGS. 9-12 are schematic illustrations of surface characterizing device systems according to embodiments of the invention,

FIGS. 13-16 illustrate schematically various aspects of a measurement buffer of embodiments of the invention,

FIGS. 17-18 illustrate schematically communication with cloud computing systems comprising a training system or a classification system according to embodiments of the invention,

FIG. 19 illustrates an embodiment where the surface characterizing device system comprises classification system,

FIG. 20 illustrates a plurality of surface characterizing device systems communicating with a cloud-based training and classification system according to an embodiment,

FIGS. 21-24 illustrate aspects of obtaining spectroscopy measurements in various embodiments of the invention,

FIGS. 25-26 illustrate an embodiment of an ATR-based surface characterizing device system according to the invention,

FIGS. 27-29 illustrate examples of spectroscopy measurements acquired by an embodiment of surface characterising device according to the invention,

FIGS. 30-31 illustrate examples of user interfaces of an embodiment of a surface characterising device according to the invention,

FIG. 32 illustrates a training method for training a classification model according to an embodiment of the invention,

FIG. 33 illustrates a classification method of characterizing a surface according to an embodiment of the invention,

FIGS. 34-35 illustrate schematic representations of a training system embodiment and a classification system embodiment, respectively, according to the invention,

FIG. 36 illustrates a schematic representation of a training module according to an embodiment of the invention,

FIGS. 37-38 illustrate various aspects of a neural network classification model according to an embodiment of the invention, and

FIGS. 39-41 illustrate various aspects of a convolutional neural network classification model according to an embodiment of the invention,

FIGS. 42-44 illustrate examples of spectroscopy measurements acquired by an embodiment of surface characterising device according to the invention,

FIGS. 45-46 illustrate examples of user interfaces of an embodiment of a surface characterising device according to the invention,

FIGS. 47-50 illustrate block diagrams of embodiments of a surface characterization system according to the invention.

### DETAILED DESCRIPTION

The following section comprises a detailed description of the invention with reference to the figures.

The description comprises nonlimiting examples of embodiments of the invention. Details such as a specific method and system structures are provided to give an understanding of embodiments of the invention. Note that detailed descriptions of well-known methods, systems, devices, circuits, components including e.g. control leads, etc. have been omitted so as to not obscure the description of the invention with unnecessary details. It should be understood that the invention is not limited to the particular examples described below, and that a person skilled in the art may choose to implement the invention in other embodiments without these specific details. Furthermore, it should be understood that the skilled person may choose to combine features of the described embodiments and of the illustrated embodiments of the invention. As such, the invention may be designed and altered in a multitude of varieties within the scope of the invention, as specified in the claims.

### Surfaces Comprising Cured Coat

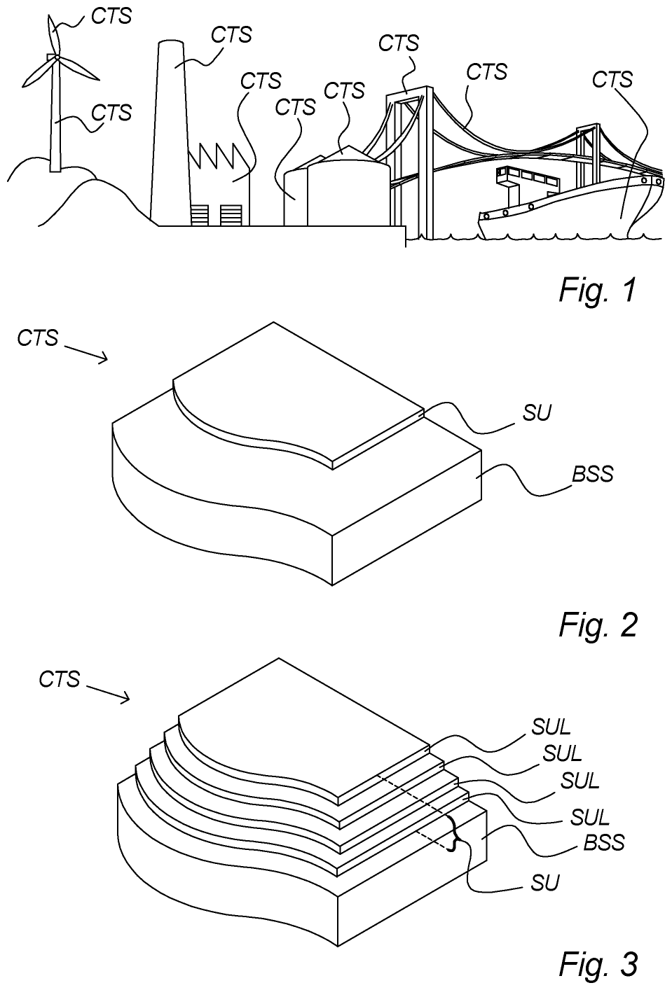

FIG. 1 illustrates examples of coated structures CTS of use cases for the invention. For example, as shown in the drawing, the invention may be advantageous for characterizing surfaces of wind turbine towers, e.g. steel towers, wind turbine blades, e.g. fibre-reinforced composites, or bridges, e.g. made of concrete or steel. For example, the invention may be advantageous in connection with industrial facilities, tanks and silos, of concrete or steel, both outside where they may be exposed to the environment, and inside where they may be exposed to various chemicals or biologic environments. For example, the invention may be advantageous in marine and offshore industries, e.g. for coatings protecting outsides of ship hulls, oil rigs and underwater constructions, such as steel, fibre-reinforced plastics or concrete, or for coatings protecting insides of ballast tanks, cargo tanks, etc. In practice, the invention may be useful and advantageous for characterising the surface of any coated structure for classifying for example the type or constituents of the coat for determining compatibility with further treatment or coating, e.g. in connection with overcoating an outdated coat. The invention may also be advantageous for characterising coating degradation and defects of a cured coat.

FIGS. 2-6 illustrate coated structures CTS and surfaces SU comprising a cured coat according to embodiments of the invention. FIG. 2 illustrates a small portion of a coated structure CTS, e.g. a ship hull or wind turbine blade. Besides a base structure BSS, e.g. steel or fibre-reinforced composite, the coated structure CTS further comprises a surface SU comprising a cured coat, for example an epoxy, polyurethane or other coat designed for the intended use of the coated structure and matched to the base structure material and the environments to which the structure is expected to be exposed. The cured coat may for example be weeks, months or years old, and the surface SU may by time have acquired other constituents in addition to the original cured coat, such as for example later treatment or deposits or residues from the environments, absorption or adhesion of water, corrosive substances, biologic or inorganic material, etc.

FIG. 3 illustrates a small portion of a coated structure CTS comprising a base structure BSS and a surface SU comprising a cured coat as in FIG. 2 and subject to the examples and comments with reference to FIG. 2 above. Further, the example in FIG. 3 illustrates that the surface SU comprising a cured coat may comprise several surface layers SUL, such as layers of cured coat. For example, a three-layer coating system may comprise, counting from the base structure BSS and out, a primer layer, a tie-coat layer and atop coat layer. Some or all surface layers SUL may be applied for technical purposes such as anticorrosive protection or fouling control, and/or for aesthetic purposes, such as colour or texture, while it is understood that colour and texture may also serve technical purposes, and technical coats may also possess decorative properties. One or more of the outer surface layers SUL constituting the surface SU comprising a cured coat may have been applied at a significantly later time than one or more of the inner surface layers SUL, e.g. in connection with maintenance, for example after several years, such as after more than 2 years from the inner surface layer.

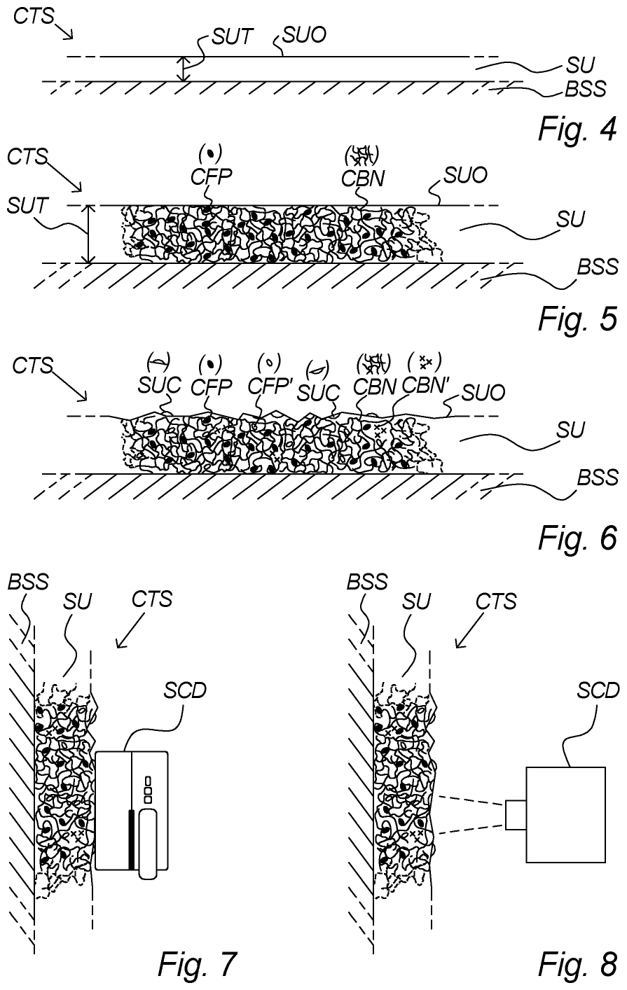

FIGS. 4-6 schematically illustrate cross-sections of coated structures CTS having a base structure BSS and a surface SU comprising a cured coat. The description and examples above with reference to FIGS. 2 and 3 also apply to FIGS. 4-6, and in particular is noted that the surface SU of FIGS. 4-6 may also comprise surface layers SUL as illustrated in FIG. 3. In FIG. 4 is defined a surface thickness SUT and a surface outside SUO of the surface SU comprising a cured coat. The surface thickness SUT of course depends on the type of coating system, the number and types of surface layers, etc. Typical surface layer dry film thicknesses of the individual cured coat layers may be 10-1000 μm, more particularly, for example, 75-400 μm, which is also the case for a single layer cured coat. As spectroscopy measurements are typically only affected by the constituents to a depth of 1.0-2.5 μm from the surface outside SOU into the surface SU comprising a cured coat, spectroscopy measurements relate solely to the outermost layer SUL, typically the top coat, of the surface SU comprising a cured coat, regardless of the coating system being a single layer or multi-layer coating system.

In FIG. 5 is illustrated schematically some of the constituents of a surface SU comprising a cured coat that has been applied relatively recently, yet with ample time to let the coating system cure, and/or has not been exposed to harsh environment. The surface SU comprising a cured coat of such relative freshness may for example comprise coat binders CBN, e.g. epoxy, polyurethane or alkyd, with more examples given above, illustrated as a web extending throughout the surface, as well as coat fillers and pigments CFP illustrated as individually distributed black particles and held in place by the web of coat binders CBN.

Over time, depending on the exposure to various harsh environments, UV radiation and heat, the surface SU comprising a cured coat may become modified as for example illustrated schematically in FIG. 6. Some of the modification may be due to additional constituents acquired from the experienced environments, whereas other modification may be due to degeneration of the constituents of the cured coat triggered by time or external factors. The surface SU comprising a cured coat of some age and/or having been exposed to harsh environments, may for example comprise the coat binders CBN and coat fillers and pigments CFP described above with reference to FIG. 5, but may now additionally comprise for example coat binder derivatives CBN′, illustrated as crosses, and coat fillers and pigments derivatives CFP′, illustrates as white particles, which have occurred due to degeneration of the coat binders CBN and coat fillers and pigments CFP. Further or instead, the relatively old or exposed surface SU may also comprise surface contaminants SUC on its surface outside SOU or which have been absorbed into the surface SU. Surface contaminants SUC acquired from various environments may for example comprise water, salts, oils, organic material such as biological material, inorganic material, etc. It is also illustrated in FIG. 6, that the surface outside SUO may, by time and/or influence of external or internal factors such as physical impact or binder decomposition, become irregular compared to the smoother surface outside SUO illustrated in FIG. 5.

A well-established concept for material analysis is spectroscopy in general, which is an overall concept encompassing a broad range of options with respect to techniques, hardware, wavelength spectrum, analysis set-up, etc.

### Surface Measuring

FIGS. 7-8 illustrate embodiments of measuring a surface in contact or from a distance. These examples are described in a context of spectroscopy measurements. However, the description also applies to devices of the invention using, for example, visible spectrum camera techniques, ultrasound measurement techniques, etc.

FIG. 7 illustrates schematically an embodiment where a surface characterizing device system SCD comprises hardware for performing a contact-based spectroscopy technique to analyse the surface SU comprising a cured coat of the coated structure CTS. Such contact-based spectroscopy sampling techniques may for example comprise various attenuated total reflection ATR techniques, where an ATR crystal is placed in contact with the surface SU to be analysed, and light, typically in an infrared spectrum, is caused to pass through the ATR crystal and reflect off the internal crystal surface that is in contact with the surface SU. An evanescent wave extends into the surface SU, for example to a depth of 1.0-2.5 μm as mentioned above. A detector is arranged to collect the reflected light as it exits the ATR crystal, and a microcontroller or other processor is configured to process the collected data into a characterization of the reflectance or absorbance of the surface SU as a function of wavelength.

When performing contact-based spectroscopy, e.g. using the ATR technique, the measurement point should advantageously be as close to the surface SU comprising a cured coat as possible, preferably in physical contact. It may therefore be beneficial to apply a pressure to the surface characterizing device system SCD, or at least its measurement point such as an ATR crystal, against the surface SU, to ensure that physical contact is achieved, as surface roughness cause contact to occur at discrete contact spots, and the number and/or area of these discrete contact spots may be increased by applying pressure. Such pressure may for example be applied by magnetic means as described further below.

A more detailed embodiment of an ATR-based surface characterizing device system SCD will be presented later herein.

FIG. 8 illustrates schematically an embodiment where a surface characterizing device system SCD comprises hardware for performing non-contact or remote spectroscopy techniques to analyse the surface SU comprising a cured coat of the coated structure CTS. Such non-contact-based spectroscopy sampling techniques may for example comprise various hyperspectral imaging techniques, where a hyperspectral imaging camera is placed at a distance from the surface SU to be analysed, and is configured to record images of the surface SU at different wavelengths.

Typically, the output of a hyperspectral imaging camera includes spatial information, such as the variation of absorbance or reflectance over a portion of the surface. The spatial information may be reduced by averaging over the spatial area, or by determining medians, etc. The different wavelengths at which images are recorded, are preferably in the infrared range, e.g. in a NIR range, MIR range, SWIR range, MWIR range, LWIR range, a functional group region IR range or an IR fingerprint region range. Preferably, the number of different wavelengths is considerably higher than the three channels recorded by normal cameras, such as at least 25 different wavelengths, at least 50 wavelengths, at least 100 wavelengths or preferably at least 200 different wavelengths within the selected IR spectrum. The surface SU comprising a cured coat may be illuminated by an infrared source with a spectrum at least covering the measurement range of the hyperspectral imaging camera, or the measurement may rely on available light comprising relevant wavelengths, for example from the sun. A calibration procedure may typically be carried out to determine a baseline of surrounding light, to which the surface reflectance can be compared.

In an example, a hyperspectral imaging camera for obtaining spectroscopy measurements suitable for the present invention may record images with 384×384 pixels at 288 different wavelengths within a NIR range of 930 nm-2500 nm at distances from the surface SU of between 5 cm and several meters, for example 1-2 meters. The spectral information for the almost 150.000 pixels may for example be reduced to a single reflectance spectrum by averaging for each of the 288 different wavelengths. In an alternative embodiment, the spatial information of at least a portion of the area is preserved during further processing, and spectrums are determined for all pixels or a number of groups of pixels in order to analyse spatial variation of surface constituents or use as additional features for training of the classification model.

In an embodiment where non-contact spectroscopy such as hyperspectral imaging is used at relatively small distances, for example at distances of 0.1 to 100 cm, e.g. 1 cm-10 cm, from the surface comprising a cured coat, the surface characterizing device system SCD may comprise a, preferably variable, spacer mounted to establish and maintain a well-defined distance during measurement. When referring to non-contact spectroscopy, use of a spacer to establish a distance between the surface and the hyperspectral imaging camera does not constitute “contact” from the perspective of the spectroscopy technique, although the spacer may be touching both the surface and the device at the time of measuring. The spacer may for example comprise a telescopic rod or a tripod to set and maintain a distance, or a telescopic or fixed-length cylinder surrounding the camera lens and infrared source to further shield the measurement from surrounding light and disturbances. Use of a spacer may also stabilize the hyperspectral imaging camera during image recording.

Embodiments where non-contact spectroscopy such as hyperspectral imaging is configured for use at relatively larger distances, for example at distances of 50 cm to 10 m from the surface SU comprising a cured coat, may be advantageous for allowing great flexibility with respect to positioning the surface characterizing device system SCD, as it is not necessary to be able to get close to the surface to be characterized. An image stabilizing technology, for example as known from normal cameras, may preferably be implemented in the surface characterizing device system SCD or its mount for handheld or drone-borne hyperspectral imaging.

It is noted, that for both the surface measurement examples in FIGS. 7-8, the surface characterizing device system SCD may be a stand-alone system consisting of a single device, or it may be a distributed system, consisting of, for example, a measurement device MD together with a smart device SD, as described in more detail below.

### Surface Characterizing Device System

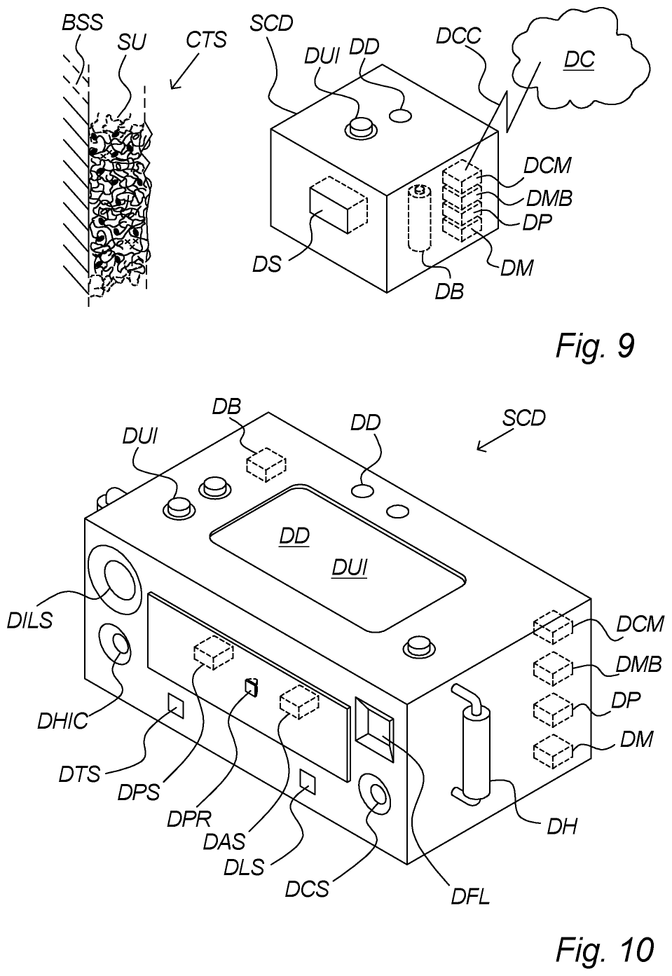

FIG. 9 is a schematic illustration of a surface characterizing device system SCD according to an embodiment of the invention, configured to make measurements ME of a surface SU comprising a cured coat, and cause representations MER of the measurements ME to be transmitted to a cloud DC, also referred to as a data cloud or cloud computing system, via a communication channel DCC. The surface characterizing device system SCD comprises a communication module DCM for transmitting the representations MER. As illustrated in the present example, the communication module DCM is configured to establish the communication channel DCC to the cloud DC itself, for example by means of Wi-Fi, GSM, LTE, 4G, 5G or other suitable data communication technologies. The communication via the communication channel DCC is preferably encrypted, and in particular the representations MER are preferably encrypted during transmission. The surface characterizing device system SCD is a portable, preferably handheld, device, and is preferably powered by a battery DB. Here the battery DB is illustrated in a recognizable battery shape, however, the battery DB may have any number of cells and any shape, such as for example a multi-cell flat and rectangular battery, and may preferably comprise a high power-density battery technology, for example Li-ion. The surface characterizing device system SCD preferably comprises a battery charger, e.g. via a USB port or an inductive charging interface. In an embodiment the battery is replaceable.

The surface characterizing device system SCD comprises a sensor DS configured to acquire measurements ME of the surface SU. The sensor DS may be arranged according to any suitable surface measuring technology, e.g. spectroscopy such as ATR spectroscopy or hyper spectral imaging, visible spectrum camera, also referred to as ‘normal’ camera, etc. The surface characterizing device system sensor DS may comprise a combination of different surface measuring technologies, for example both an ATR spectroscopy module and a ‘normal’ camera, or both a hyperspectral imaging camera and a ‘normal’ camera, etc. Some possible options for sensor DS are described in more detail later herein.

A processor DP and memory DM are included in order to control the sensor DS, receive and process the measurement ME, and prepare a representation MER of the measurement for transmission by the communication module DCM. The representation MER may simple be identical to the measurement ME, or may optionally be converted into a different data format, be compressed, be pre-processed for training, be enhanced with metadata or other information, etc. In an embodiment, the representation MER includes a label designating a surface characterizing class SCC associated with the measurement ME and surface SU.

Also a user interface DUI and a visual output device DD, such as an LED or display, is provided to allow an operator DO to control the surface characterizing device system such as when to measure, and to indicate a measurement or connection status. In a simple embodiment, the user interface DUI may consist of one or more buttons or knobs, and the visual output device DD simply be one or more LEDs or a single-row LCD display. This may for example be sufficient for an extremely user-friendly point-and-shoot version of the surface characterizing device system SCD, where the operator DO only has to point the sensor DS of the surface characterizing device system SCD towards the surface SU and/or touch the surface SU with the sensor DS, depending on the sensor technology as mentioned above, and press a start-button DUI. A measurement or connection status may be shown by a color or blink code of a simple LED, forming the visual output device DD in this example. In an embodiment, the operator may in connection with performing a measurement ME input a label designating a surface characterizing class SCC associated with the measurement ME and surface SU.

The surface characterizing device system SCD comprises a measurement buffer DMB, which may be implemented as part of the memory DM or as a separate memory. The measurement buffer DMB is configured to temporarily store representations MER of the measurements ME of the surface SU in case the communication module DCM is prevented from establishing the communication channel DCC. When the communication channel DCC is reestablished, the temporarily stored representations MER may be read from the measurement buffer DMB and transmitted by the communication module DCM. The representations MER may be encrypted when temporarily stored in the measurement buffer DMB so they are ready to transmit without further processing, or encryption may be applied only during transmission. The representations MER may be stored in a compressed format in the measurement buffer in order to reduce storage space requirements. Compressed representations MER may also be transmitted in compressed format to reduce communication requirements.

As the surface characterizing device system SCD is preferably a portable system intended to be brought near a surface SU of a coated structure CTS in order to make the measurements ME, and as these surfaces SU may be located inside the coated structures CTS with base structures BSS of for example concrete or steel, the circumstances for wireless communication may not be optimal. Thus, the measurement buffer DMB is provided to temporarily store acquired measurement ME when communication is prevented.

FIG. 10 is a more detailed, still schematic, illustration of a surface characterizing device system SCD according to an embodiment of the invention. It is noted that elements in the drawings are not necessarily drawn to scale, nor necessarily with their actual shapes, proportions or locations. Further, it is noted that the surface characterizing device system SCD in FIG. 10 includes several optional features which are not all necessary to implement according to the present invention.

The embodiment of FIG. 10 comprises some of the same features as described above with reference to FIG. 9, and with the same purposes, for example a processor DP and memory DM, a measurement buffer DMB, a communication module DCM and battery DB.

The user interface DUI and visual output device DD in this example embodiment comprises a touch display, in order to provide detailed information to an operator DO, and allow advanced input in form of text or touch gestures, e.g. swipe, pinch, etc. Buttons and LEDs may also be provided for parts of the user interface DUI and visual output device DD, or may be considered obsolete when a touch display is implemented.

In the example of FIG. 10, the surface characterizing device system SCD is shown with handles DH for facilitating carrying, holding, pressing and positioning the device relatively to the surface SU to be measured. In other embodiments, there are no handles, or the handles DH are replaced with, or supplemented with, brackets for mounting on a camera tripod, a robot or drone DR, etc.

With respect to sensor DS, the embodiment of FIG. 10 comprises several options. Various embodiments may comprise any number of these, or any combination of these, as well as other sensors.

One of the example sensors DS illustrated in FIG. 10 comprises a camera sensor DCS, such as a visible spectrum camera, optionally supplemented by a flash light DFL, preferably LED-based. By means of a ‘normal’ camera sensor DCS, the surface characterizing device system SCD may produce measurements ME consisting of images of the surface. This may for example be advantageous in order to recognize, for example by means of artificial intelligence image recognition, damages of the coat, for example cracks, scratches, corrosion, blisters, etc.

Another example sensor DS comprises a hyperspectral imaging camera DHIC, optionally supplemented with an infrared flash light DIFL. This sensor technology may for example be advantageous for acquiring spectroscopy measurements ME from a distance of the surface SU comprising a cured coat. This technology is described in more detail elsewhere herein.

An advantageous example sensor DS of an embodiment comprises a contact-based spectroscopy sensor, such as for example an ATR-spectroscopy sensor, comprising a prism DPR arranged with one of its flat facets parallel with an outside of the surface characterizing device system SCD, and preferably slightly protruding, in order to be pressed against the surface SU to be measured. Contact-based spectroscopy and embodiments thereof are also described in more detail elsewhere herein.

In an embodiment, the sensor DS comprises an ultrasound emitter and an ultrasound receiver to characterize the surface SU by comparing the transmitted and received ultrasound.

The surface characterizing device system SCD may further comprise a number of supplementary sensors, for example a temperature sensor DTS and/or light sensor DLS to facilitate calibration or measurement validity checks, a pressure sensor DPS particularly advantageous in an embodiment implementing ATR spectroscopy to validate or control a suitable pressure of the prism DPR against the surface SU, and an accelerometer sensor DAS which may among others be used for measurement validation by indicating when the surface characterizing device system is not held sufficiently steady for measuring. Further or different supplementary sensors may be implemented, for example a humidity sensor, a geolocation sensor such as GPS, a text scanner for reading identification numbers or names, a scanner for reading bar codes, QR codes, RFID tags or NFC tags in or on the surface for obtaining an ID or metadata about the surface, etc.

As the measurement buffer DMB is implemented for temporarily storing measurement representations MER when it is not possible to transmit them via the communication module DCM, it may be advantageous to implement a filtering or pre-validation of measurements in order to not fill up the measurement buffer DMB with useless measurements, and risk not having room for all the relevant measurements. This may be particularly challenging when the loss of connection is unexpected, so the amount of planned measurements cannot have been coordinated with the amount of storage space in the measurement buffer before staring the measuring session. In a preferred embodiment, the processor DP is configured to evaluate and discard measurements ME that are not considered suitable for the purpose, such as training, test and/or classification. The evaluation of a measurement ME or measurement representation MER may comprise considering for example the dynamic range of the measurement values (if they are all unrealistically close in level), the maximum and minimum levels (if it is basically just the noise floor or baseline that has been picked up), and other statistical or signal analysis procedures, possibly involving comparing with pre-defined expected thresholds, patterns, etc. For example, when an image taken with a camera sensor only comprises black color throughout all its pixels, it may be considered an erroneous measurement unsuitable for surface characterization, and can be discarded to not fill up the measurement buffer DMB. In an embodiment, the evaluation or validation of measurements further comprises input from a supplementary sensors. For example may a light sensor DLS indicate whether there, at the time of measuring, was sufficient light for a non-contact sensor, or whether it was sufficiently dark, indicating good contact for contact-based sensors. For example may a pressure sensor DPS provide an indication whether the measurement was acquired while sufficient pressured on the surface was applied by the surface characterizing device system. For example may an accelerometer sensor DAS provide an indication whether the surface characterizing device system SCD, and thereby the sensors DS, was held sufficiently steady during measurement. The surface characterizing device system SCD may be configured to inform the operator DO, for example by means of the visual output device DD, that a measurement ME has been discarded, and for what reason, in order for the operator DO to repeat the measurement.

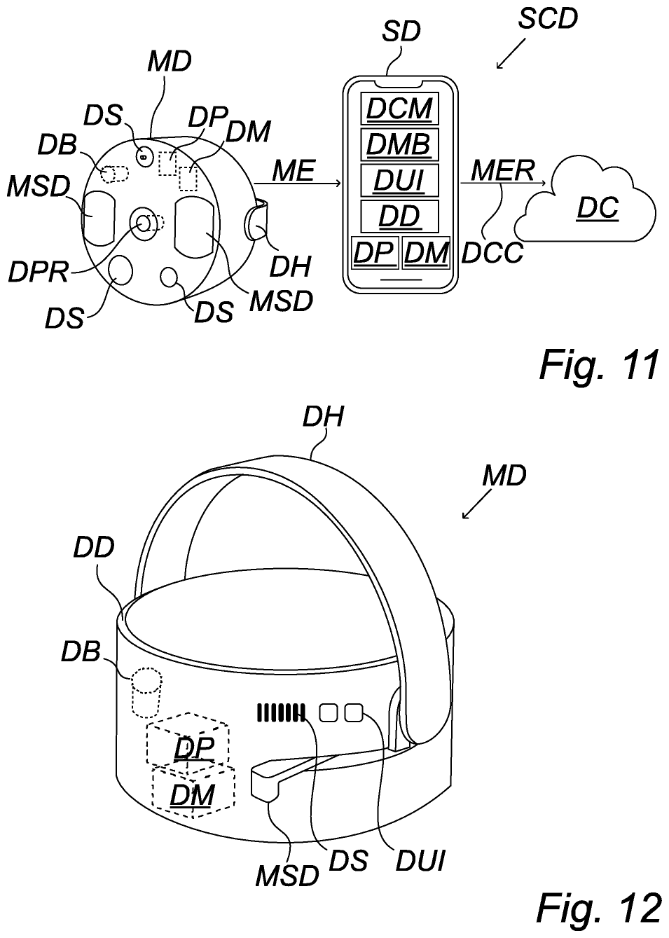

FIGS. 11-12 is a schematic illustration of a distributed surface characterizing device system SCD according to an embodiment of the invention. The surface characterizing device system SCD is as described above configured to make measurements ME of a surface SU comprising a cured coat, and cause representations MER of the measurements ME to be transmitted to a cloud DC, also referred to as a data cloud or cloud computing system, via a communication channel DCC.

The distributed surface characterizing device system SCD of FIGS. 11-12 comprises a measurement device MD and a smart device SD, where the measurement device MD is configured to obtain the measurements ME and transfer them to the smart device SD, such as a smartphone, tablet computer or laptop, by a local communication means such as Bluetooth, WiFi or a USB-cable, and the smart device SD is configured to transmit the representation MER of the measurement ME, such as in a certain format, or with added metadata or other measurements, via the communication channel DCC to the cloud DC.

The measurement device MD comprises at least one sensor for making a measurement ME. In this example, the measurement device MD comprises an ATR-spectroscopy sensor based on a prism DPR arranged with one of its flat facets parallel with an outside of the measurement device MD.

In this example, a magnetic switchable device MSD is implemented in the measurement device MD to ensure a proper attachment to the surface to be measured, such as ensuring a proper pressure of the prism DPR against the coat. A magnetic switchable device MSD, also referred to as mechanically switchable magnets, may comprise a system of two or more permanent magnets arranged so that they can be rotated or displaced relative to each other, and thereby toggle the magnetic attachment force as experienced. An example of location of magnetic attachment points is shown in FIG. 11, on each side of the prism DPR, and a lever to displace or rotate the magnets to toggle the magnetic flux is shown in FIG. 12. Such a purely mechanical, manual system consumes no electric power from the battery DB and is easily operated by a user even for strong attachment.

The measurement device MD in this example further comprises a battery DB and a handle DH for portability and facilitating handling and positioning at the surface. A charging port for charging the battery DB, and/or a means for replacing the battery may also be provided. The measurement device MD may comprise a processor DP and a memory DM. In this distributed embodiment, the smart device SD may typically comprise powerful processing means and large storage capacity, whereas the measurement device MD may in an embodiment simply comprise such features to the extent necessary to control the sensors and transfer measurements to the smart device SD for further processing.

A simple version of user interface DUI and visual output device DD may also be sufficient in the measurement device MD in this embodiment where the surface characterizing device system SCD is distributed between the measurement device MD and smart device SD, as the latter may typically have powerful graphical capability and versatile, user-friendly user interfaces.

In the example, the measurement device MD may also have more sensors DS, such as sensors for temperature, humidity, dry film thickness, etc.

The smart device SD may typically comprise several long-range communication technologies, and is thereby advantageous to use for establishing the communication channel DCC. The smart device SD thereby comprises the communication module DCM, for example providing for mobile data connections through GSM, LTE, 3G, 4G, 5G, etc., and WiFi, etc.

The smart device SD also comprises a battery, although not shown in the drawing.

Further, the smart device SD typically comprises powerful graphics and a large and user-friendly touch display which may be used for both user interface DUI and visual output device DD. The measurement device MD may simply have a few status LEDs to indicate power state, battery state, ready state, or the like, and a simple on/off button, and possibly a measurement start button, as all detailed information to the user, and all detailed input from the user may preferably be communicated by the touch screen of the smart device SD.

A software application, also referred to as an app, may be installed on the smart device SD to control its processor DP to further control the measurement device MD, such as activating sensors, performing measurements, making settings, and viewing received measurements.

A smart device SD such as smartphone or tablet computer for controlling the surface characterizing device system SCD may in some embodiments be mounted physically on the measurement device MD, or may for example be carried separately by the operator DO.

As mentioned above, the communication between the measurement device MD and the smart device SD uses a short-range communication module using a technology such as Bluetooth, BLE, WiFi, WiFi Direct, cable such as USB cable, etc. This enables the smart device SD to control the measurement device MD in reaction to user commands in the user interface DUI, and to receive the measurements ME from the sensors of the measurement device MD.

The description of further details and features mentioned above in relation to FIGS. 9-10 also apply to the embodiment of FIGS. 11-12. For example the measurement buffer DMB described above as part of the surface characterizing device system SCD may preferably be implemented in the smart device SD to accommodate representations MER of measurements which have not been transmitted to the cloud DC yet, for example due to bad connection status.

In various embodiments, the distribution of features between measurement device MD and smart device SD may also be different that described above, for example leaving more of the processing to the measurement device MD, or using sensors of the smart device SD for further measurements ME, e.g. using a visual spectrum camera or a GPS of the smart device to provide measurements ME or metadata.

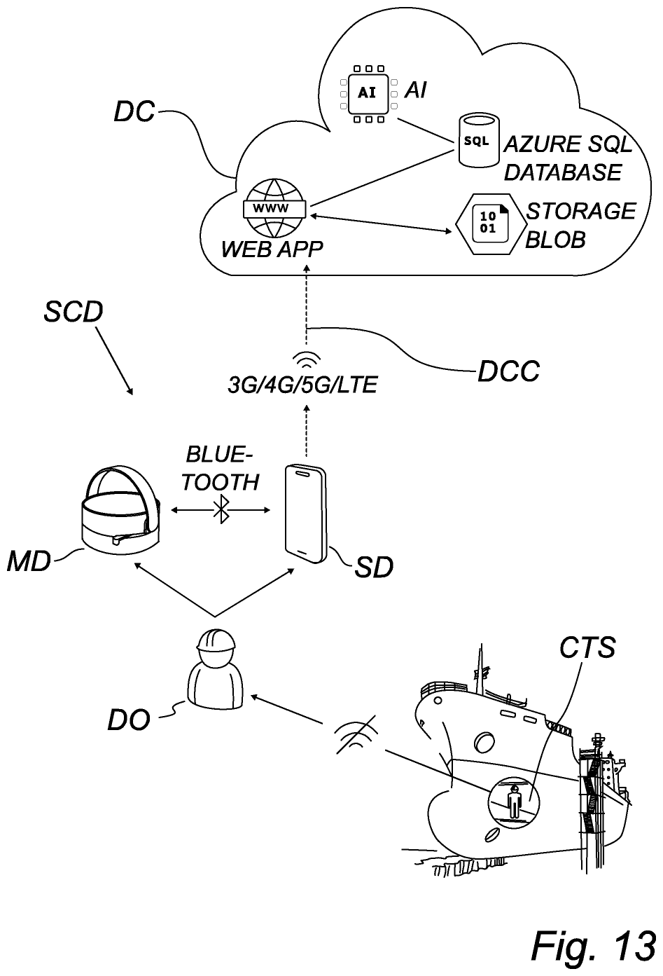

FIG. 13 illustrates an embodiment of a surface characterizing device system SCD in a scenario where a communication channel DCC cannot be established during measurement. In this scenario, the communication channel DCC may for example be lost, as indicated by the crossed connection system, a coated structure CTS to be measured is located deep into a cargo hold or tank of a steel vessel, where electromagnetic waves are shielded by the steel and long-range electromagnetic communication becomes impossible. The drawing also depicts a distributed embodiment as described above, with a measurement device MD and a smart device SD who communication internally with each other by short-range communication, such as Bluetooth in this example. During measurement when the operator DO is in the limited communication location inside the ship, the measurement device MD and smart device SD can still communicate with each other to obtain one or more measurements. The measurements are stored in the measurement buffer of the smart device SD as long as the long-range communication is not possible.

When the operator DO takes the surface characterizing device system SCD out of the electromagnetic shield, the communication channel DCC can be established for long-range communication, for example using 4G or 5G, for example via the internet. Thereby the smart device SD can transmit the representations of measurements, together with any assigned metadata, to the cloud DC. In this connection is it relevant to note, that the metadata and measurements need to be synchronized to reflect the state when the measurement was taken at the relevant coated structure CTS, and not the state at the time of upload to the cloud. This may for example be particularly important when assigning metadata such as GPS location or other location information, e.g. “tank 4, starboard side”, or assigning information about the coat as observed by the operator DO.

The drawing further illustrates some of the elements of the cloud DC, such as a web app to facilitate and control the communication, e.g. by an API, and various storage locations and AI facilities to train, test and expose a classification model for identifying coats or the like as described herein.

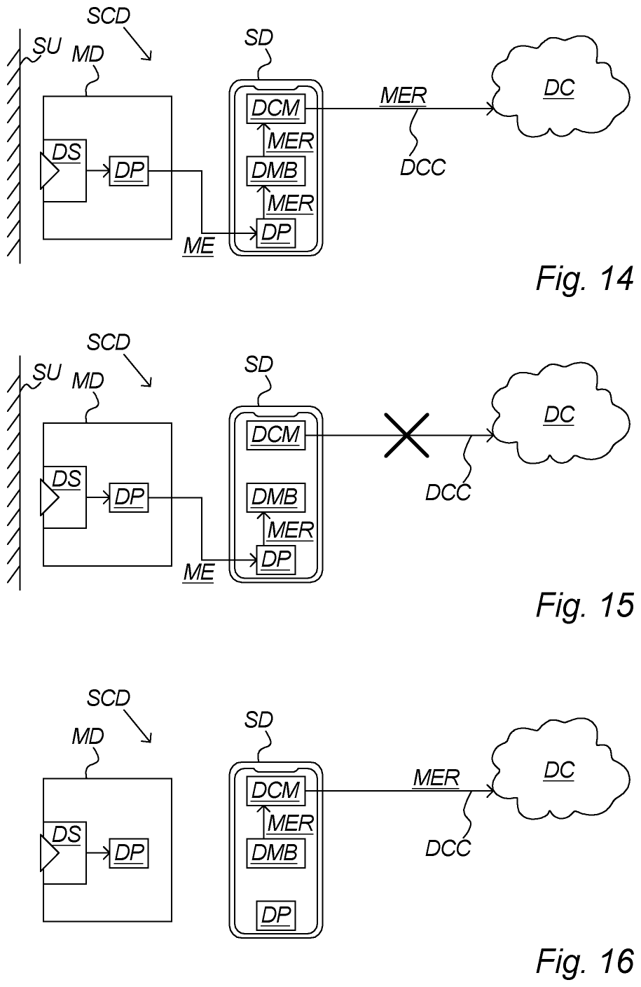

FIGS. 14-16 illustrate schematically various aspects of the measurement buffer DMB of embodiments of the invention. FIGS. 14-16 illustrate a surface characterizing device system SCD, here shown as a distributed embodiment having a measurement device MD and a smart device SD, but other described embodiments may utilize a measurement buffer DMB in similar ways. It is configured to employ a sensor DS to acquire a measurement ME of a surface SU comprising a cured coat. The sensor DS may for example comprise a contact-based spectroscopy configuration such as ATR with a prism. The sensor DS may also or instead comprise other surface measurement configurations. In the drawing is shown a distance between the surface SU and the sensor DS to not cramp the drawing too much, whereas, depending on the measurement technology, there may in practice be contact between the sensor DS and the surface SU, and even preferably attachment, e.g. by magnetic force. For simplicity, various features described elsewhere of the surface characterizing device system SCD has been left out of these drawings, such as battery DB, user interface DUI and visual output device DD, memory DM, further sensors DS, etc.

FIG. 14 illustrates that a communication channel DCC is established between the communication module DCM and a cloud DC, also sometimes referred to as data cloud, cloud computing system or data center. The communication channel DCC may be established by means of any data communication technology, wired or wireless. Measurements ME may thus be processed by the processor DP into representations MER of the measurements ME and be transmitted to the cloud DC via the established communication channel DCC. In this situation the measurement buffer DMB is not necessarily be employed. In an embodiment as shown, all representations MER of measurements are sent through the measurement buffer DMB also when the communication channel DCC is working, but in other embodiment, the representations MER of measurements are sent directly from processor DP to communication module DCM when the communication channel DCC is working during measurement.

FIG. 15 is the same embodiment as FIG. 14, but now illustrates that the communication channel DCC is broken. This may refer to any of not being possible to establish the communication channel DCC in the first place for both expected and unexpected reasons, or to an initially established communication channel losing the connection, e.g. as shown in FIG. 13 above. Expected reasons may for example comprise travelling to an isolated location, e.g. an offshore wind farm for measuring wind turbine blade surfaces, where it is known beforehand that there will not be a kind of network coverage compatible with the communication module DCM, for example lack of 4G or 5G mobile data coverage. Unexpected reasons may for example comprise losing a communication channel connection when proceeding deeper into a ballast tank interior where electromagnetic waves are shielded by the steel tank and the communication suddenly stops working. In both cases, the measurement representations MER may according to the invention be stored temporarily in the measurement buffer DMB instead of being transmitted. Locations where it is not possible to establish the communication channel is also referred to as a first environment herein.

FIG. 16 is the same embodiment as FIGS. 14-15, but now illustrates that the communication channel DCC is established again, for example when the surface characterizing device system SCD gets back to compatible network coverage, or when the surface characterizing device system SCD gets close enough to a tank entrance to pick up sufficient signal again. For example, when the surface characterizing device system SCD leaves the first environment and enters a second environment different from the first environment. Hence, the surface SU is not shown in this figure. The measurement representations MER that were stored temporarily in the measurement buffer DMB, may now be transmitted to the cloud DC from the measurement buffer DMB according to the invention.

In all of FIGS. 14-16 is illustrated the task of transmitting a representation MER. This is just one example of using the measurement buffer DMB. In various embodiments, the measurement buffer DMB may be employed in the same way when attempting communication of other information and it turns out the communication channel is not or cannot be established at the moment. In particular it is noted, that the measurement buffer DMB may be employed for temporarily storing representations MER of measurements ME that are going to be used as training input measurements TIM for a training system TS, and/or be employed for temporarily storing representations MER of measurements ME that are going to be used as input measurements IM for a classification system CS.

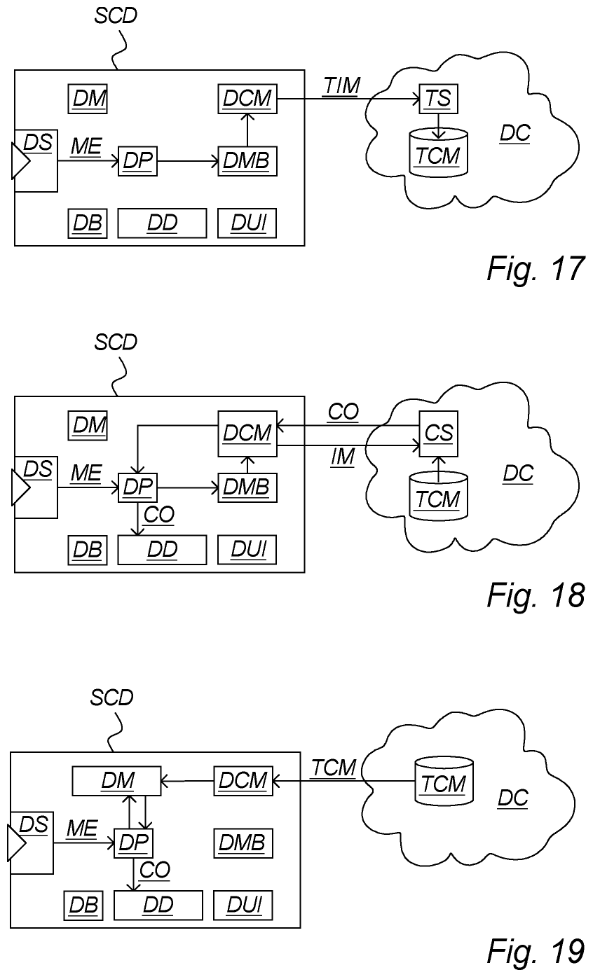

Examples of this are given in FIGS. 17-19. For these examples are shown a surface characterizing device system SCD that is not distributed, but instead is a single device system comprising both sensors DS and communication module DCM in the same device SCD. However, as above, the functionality described with reference to these drawings work in a similar way for a distributed system comprising a measurement device MD and a smart device SD.

FIG. 17 illustrates schematically that the cloud computing system DC may comprise a training system TS configured to provide a trained classification model TCM for characterizing surfaces SU comprising cured coats. The training system TS and trained classification model TCM may be stored and executed anywhere, including dedicated private servers, private clouds or public clouds, but is drawn as a generic cloud solution for simplicity. In FIG. 17 is illustrated that the surface characterizing device system SCD transmitted a training input measurement TIM to the training system TS, in other words training data for the training of classification model, as described elsewhere herein. The training input measurement TIM may preferably be derived from the measurement ME of the surface SU, for example in form of the measurement representation MER described above.

As described above, the training input measurement TIM may advantageously be temporarily stored in the measurement buffer DMB when the communication channel DCC between the communication module DCM and the cloud DC is not established. As described above, the measurement buffer DMB need not necessarily be used when a communication channel DCC is established.

FIG. 18 illustrates schematically that the cloud computing system DC may comprise a classification system CS configured to use a trained classification model TCM to characterize surfaces SU comprising cured coats. The classification system CS and trained classification model TCM may be stored and executed anywhere, including dedicated private servers, private clouds or public clouds, but is drawn as a generic cloud solution for simplicity. In FIG. 18 is illustrated that the surface characterizing device system SCD transmitted an input measurement IM to the classification system CS, in other words input data for classifying a surface, as described elsewhere herein. The input measurement IM may preferably be derived from the measurement ME of the surface SU, for example in form of the measurement representation MER described above. The classification system CS may return a classification output CO, for example comprising a surface characterizing class SCC associated with the surface SU that was measured, and the classification output CO may for example be shown on the visual output device DD. In an embodiment, the classification output CO may instead, or in addition, be transmitted from the classification system CS to a cloud storage for central access and storage.

As described above, the input measurement IM may advantageously be temporarily stored in the measurement buffer DMB when the communication channel DCC between the communication module DCM and the cloud DC is not established. As described above, the measurement buffer DMB need not necessarily be used when a communication channel DCC is established. The cloud DC may also comprise a classification output buffer for temporarily storing classification outputs when access to a requesting surface characterizing device system is temporarily lost.

FIG. 19 illustrates schematically that the cloud computing system DC may comprise a trained classification model TCM for characterizing surfaces SU comprising cured coats. The trained classification model TCM may be retrieved to the surface characterizing device system SCD and for example be stored in the memory DM. The processor DP may be configured to execute a classification system CS to directly and locally classify measurements ME by means of the retrieved trained classification model TCM. The local classification system CS implemented by the processor DP may return a classification output CO, for example comprising a surface characterizing class SCC associated with the surface SU that was measured, and the classification output CO may for example be shown on the visual output device DD. In an embodiment, the classification output CO may instead, or in addition, be transmitted from the surface characterizing device system SCD to a cloud storage for central access and storage. In an embodiment, the trained classification model TCM retrieved to the surface characterizing device system SCD is a simplified model, for example with less parameters that a complete trained classification model TCM stored in the cloud, thereby requiring less memory DM and less processor DP resources in the, preferably, portable, battery powered surface characterizing device system SCD. In an embodiment, the locally stored trained classification model TCM may be used as backup to validate measurement ME when the communication channel DCC is not established for any reasons, for example the reasons mentioned above, and the measurements ME or measurement representations MER are also stored in the measurement buffer DMB for later processing by the central classification system CS for validation and or further training of the classification model.

It is again emphasized, that the embodiments of FIGS. 17-19 may be implemented with the distributed configuration as described above.

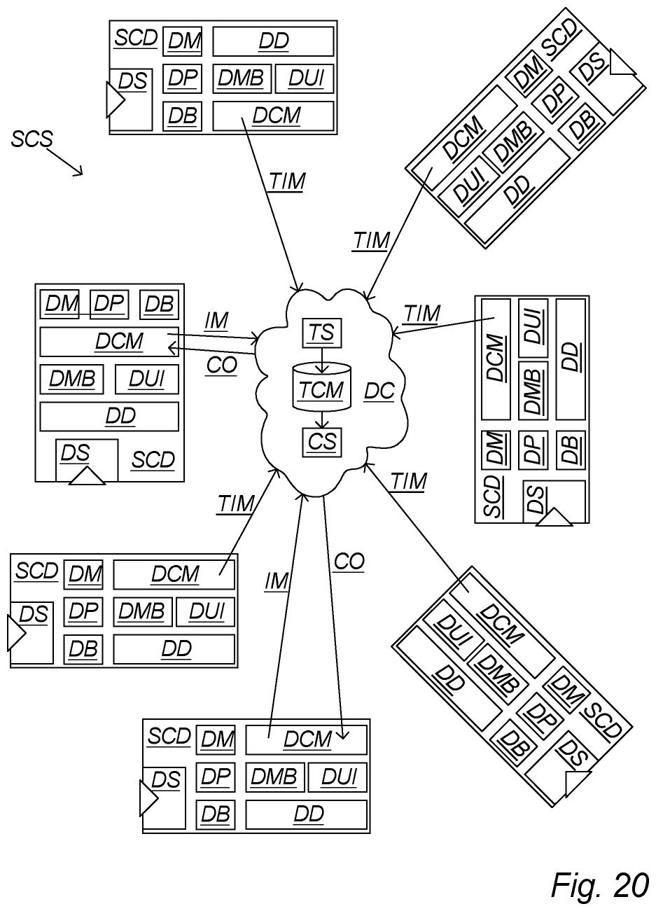

FIG. 20 illustrates schematically a surface characterization system SCS comprising a plurality of surface characterizing device systems SCD of any of the various embodiments described above. They can all be of the same configuration, or a mix. In other words, some or all the surface characterizing devices SCD may be distributed systems comprising measurement devices MD and smart devices SD. This is not shown in this drawing for simplicity.

A cloud computing system DC comprises a training system TS, a trained classification model TCM and a classification system CS. A subset of the surface characterizing device systems SCD are performing measurements ME of surfaces SU as described, and are transmitting the results as training input measurements TIM to the cloud DC for use in the training system TS. A subset of the surface characterizing device systems SCD are performing measurements ME of surfaces SU as described, and are transmitting the results as input measurements IM to the cloud DC for classification in the classification system CS, and are in return receiving classification outputs CO. In both cases, the measurement buffers DMB of the surface characterizing device systems SCD may advantageously be utilized whenever the connection with the cloud DC is lost or not possible to establish, for temporary storage of training input measurements TIM and/or input measurements IM until connection with the cloud DC can be established.

It is noted that for all of the embodiments described herein, the exchange of measurements, classification outputs, trained classification models and other information, may be conducted between the surface characterizing device systems SCD and the central training system TS or classification system CS, for example by means of communication channels DCC established from communication modules DCM in the surface characterizing device systems, and preferably via the Internet. Further, they may be conducted directly from a single-device surface characterizing device system SCD, or when distributed, through a smart device SD, for example a smartphone, tablet computer, laptop computer, smart watch, etc. and preferably via the Internet. Likewise, the processing of measurements, the assigning or confirmation of training input labels, or classification using a downloaded trained classification model, may be performed using a processor DP and memory DM of the surface characteristic device SCD, or by distributing these tasks to a smart device SD, for example a smartphone, tablet computer, laptop computer, smart watch, etc.

### Measuring Surfaces

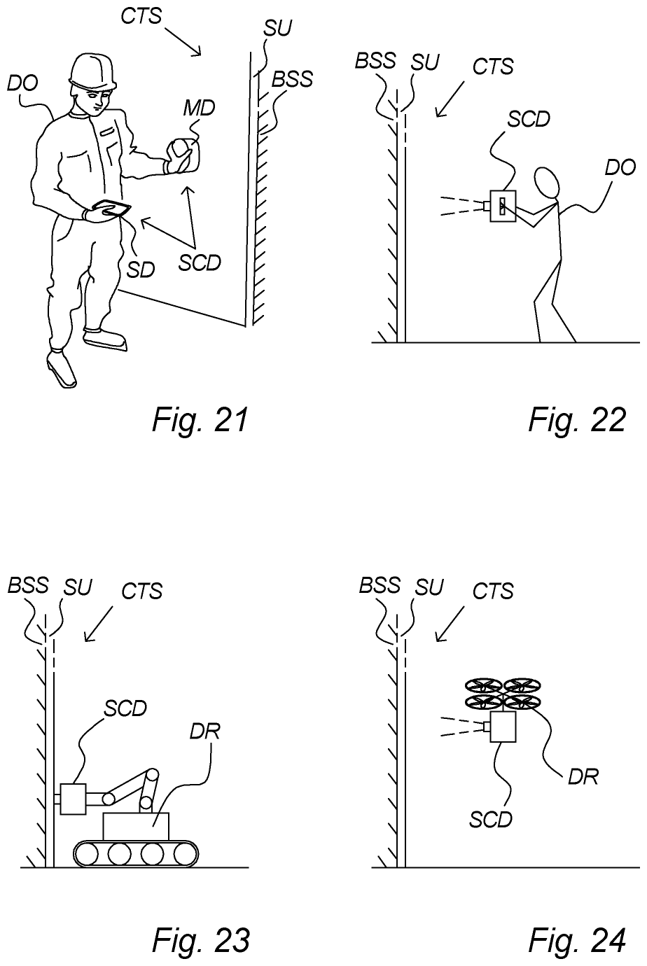

FIGS. 21-24 illustrate various embodiments of operating a surface characterizing device system SCD to characterize a surface SU comprising a cured coat on a base structure BSS of a coated structure CTS. FIG. 21 illustrates a distributed system with a human device operator DO holding a measurement device MD of a surface characterizing device system SCD against the surface SU comprising a cured coat, in order to obtain a spectroscopy measurement ME, and also carrying a smart device SD of the system for communicating the measurements. In this embodiment, the surface characterizing device system SCD may for example comprise ATR technology for performing contact-based spectroscopy, e.g. as shown in FIGS. 11-12 and 25-26.

FIG. 22 illustrates the human device operator DO holding a surface characterizing device system SCD in free space and with a measurement portion pointed towards the surface SU comprising a cured coat. In this embodiment, the surface characterizing device system SCD may for example comprise hyperspectral imaging camera technology for performing non-contact spectroscopy as described above.

FIG. 23 illustrates a ground-based drone or robot DR operating a surface characterizing device system SCD for performing a contact-based spectroscopy measurement. With a stable ground grip, the ground-based drone or robot DR may be able to provide a sufficient pressure of the surface characterizing device system SCD against the surface SU comprising a cured coat to achieve good spectroscopy measurements. A ground-based drone or robot DR may in an embodiment be used for operating a non-contact spectroscopy-based surface characterizing device system SCD by keeping a suitable distance, as described above, to the surface SU. Although the ground-based drone or robot DR is illustrated as a tracked vehicle, it may alternatively have wheels or leg mechanisms or any other propulsion solution. A ground-based drone or robot DR may also be referred to as an unmanned ground vehicle UGV.

FIG. 24 illustrates an airborne drone or robot DR operating a surface characterizing device system SCD for performing a non-contact spectroscopy measurement. An airborne drone or robot DR has extreme access and positioning flexibility as it may basically go anywhere with free space, in particular when implemented as a quadcopter, i.e. a helicopter with four rotors, and with automatic stabilization, thereby allowing acquisition of non-contact spectroscopy measurements at surfaces difficult to reach. An airborne drone or robot DR may in an embodiment be used for operating a contact-based spectroscopy surface characterizing device system SCD by applying propulsion force against the surface SU comprising a cured coat, although it may be difficult to achieve the same degree of pressure achievable by a ground-based drone or robot DR of FIG. 22. The airborne drone or robot DR is illustrated as a quadcopter but may alternatively comprise any other airborne propulsion solution such as other rotor configurations. An airborne drone or robot DR may also be referred to as an unmanned aerial vehicle UAV. In an embodiment, the surface characterizing device system SCD is transferred to its measurement position by an underwater drone, also sometimes referred to as an unmanned underwater vehicle. With underwater drones for propulsion, it may be possible to acquire spectroscopy measurements of for example sub-surface portions of ship hull coating or underwater constructions without the need of divers.

The above-described drones or robots DR and various related embodiments may also be referred to as remote operated vehicles ROV when they are operated from a remote control-station, typically by a human device operator DO. In other embodiments, the drones or robots DR may be autonomous vehicles to automatically transport the surface characterizing device system to a predetermined or dynamically selected target location, perform a measurement and return or proceed to a next target location. Various remote operated vehicles or autonomous vehicles may be employed to carry the surface characterizing device system SCD to a desired measurement location, or the surface characterizing device system SCD may have built-in remote operated vehicle or autonomous vehicle propulsion and control modules.

### Spectroscopy

It should be understood that the implementation of the invention is not limited to any particular type of surface measurement technology. Preferred measurement technologies comprise various kinds of spectroscopy measurements, including types of spectroscopy measurements that is not exemplified in the following sections. Hence following, that the invention can be implemented to utilize different wavelengths spectra, different spectroscopy acquisition techniques, etc., or other surface measuring technology.

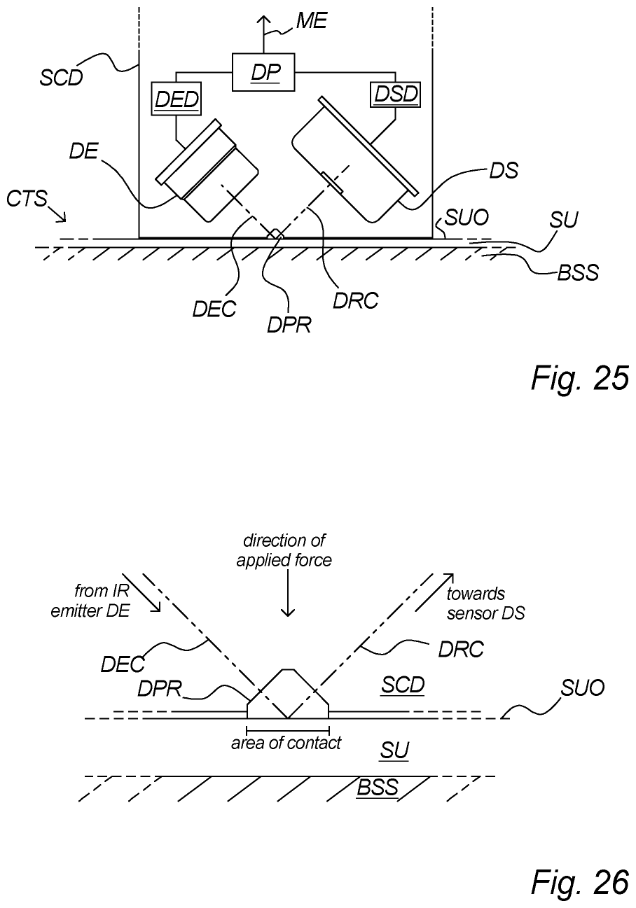

FIG. 25 illustrates an embodiment of a surface characterizing device system SCD with attenuated total reflection ATR technology for performing spectroscopy measurements ME. FIG. 26 is a close-up view of the crystal and area of contact. It is noted that the drawings are schematic illustrations and block diagrams, and in particular is noted that it is not drawn to scale with respect to crystal size compared to layer thickness or the overall size of a surface characterizing device system SCD. It is further noted, the these drawings do not include all features and elements of the surface characterizing device system SCD, hence, the dashed lines in FIG. 25, and that the surface characterizing device system SCD can be any of the single-device or distributed device embodiments.

At the core of the spectroscopy measuring part of the surface characterizing device system SCD is an ATR prism DPR, also referred to as an ATR crystal, configured for single-bounce ATR. The prism DPR is mounted at one of the outside surfaces of the surface characterizing device system SCD and is preferably protruding slightly from the surface for facilitating good contact between the prism DPR and the surface SU comprising a cured coat in an area of contact when the surface characterizing device system SCD is pressed in a direction of applied force against the surface SU. These aspects are most clearly seen in FIG. 26. The prism DPR may for example be an optical grade diamond prism, for example with a rectangular contact surface, for example with a contact surface of 2×2 mm, and a height of 1.2 mm. The angles between the two facets where the emitted IR beam DEC enters and the reflected IR beam DRC exits, respectively, may preferably be 90°, so that the angle between the surface SU and any of the IR beams becomes 450 and so that the IR beam only experiences a single bounce or reflection before exiting the prism. The skilled person in the field of ATR spectroscopy may envisage other suitable prism DPR configurations within the scope of the invention, for example with different prism shape or dimensions, configured for more than one bounce, etc.

An emitted IR beam DEC may be produced by an infrared emitter DE being an infrared source for a suitable wavelength range with respect to the desired spectrum of analysis. For example, the infrared emitter DE may be a thermal MEMS-based infrared source for electrical modulation and infrared radiation in the range of 2,000-14,000 nm, thereby covering a large part of the IR spectrum relevant in spectroscopy applications. The infrared emitter DE may for example be an EMIRS200 from the Swiss company Axetris AG packaged in a T039 package with reflector 2, with a focal length between 0 and 7 mm. This is an electrically modulated thermal infrared emitter based on a resistive heating element integrated onto a thin dielectric membrane which is suspended on a micro-machined silicon structure. The infrared emitter DE is mounted in the surface characterizing device system SCD so that the emitted IR centreline DEC is perpendicular to, and points towards, one of the facets of the prism DPR as illustrated in FIGS. 25 and 26. The surface characterizing device system SCD further comprises an emitter driver DED for driving the infrared emitter DE.

A reflected IR beam DRC may be sampled by a sensor DS configured to detect the reflected IR beam DRC intensity in a desired range of wavelengths within the range produced by the infrared emitter DE. For example, the sensor DS may be a spectrally tuneable pyroelectric detector, for example having tuneable Fabry-Perot filter to scan through different wavelengths in the desired range, and for example the desired range may be 8,000 nm to 10,500 nm. For example, the sensor DS may provide for measuring at least 50, such as at least 100, and more preferably at least 200, for example 250, individual wavelengths within the range. The sensor DS may for example be an LFP-80105C-337 from the German company InfraTec GmbH packaged in a TO8 package with a Fabry-Perot filter tuneable in the range of 8,000 nm to 10,500 nm and a filter time constant of 1-8 ms, or an LFP-5580C-337, also from InfraTec GmbH, packaged in a TO8 package with a Fabry-Perot filter tuneable in the range of 5,500 nm to 8,000 nm and a filter time constant of 2-10 ms. These sensors allow for quick tuning of the optical filter over a defined spectral range by simply adjusting the control voltage to monitor a broad spectral region. In one embodiment the sensor is controlled to perform a measurement at 250 individual wavelengths within its range, thus measuring the reflected IR beam DRC for each 10 nm from 8,000 to 10,500 nm. In another embodiment the measurement is performed for each 10 nm from 5,500 to 8,000 nm. The sensor DS is mounted in the surface characterizing device system SCD so that the reflected IR centreline DRC is perpendicular to both one of the facets of the prism DPR and the aperture of the sensor DS as illustrated in FIGS. 25 and 26. The surface characterizing device system SCD further comprises a sensor driver DSD for driving the sensor DS. In embodiments using infrared emitters DE and/or sensors DS with limited wavelength range, a broader range or combination of ranges may be achieved by implementing two or more infrared emitters DE and/or sensors DS, possibly sharing the same prism DPR, or implementing a prism DPR for each sensor DS. With the example infrared emitter DE mentioned above, EMIRS200, having a range of 2,000-14,000 nm, a single infrared emitter DE and a single prism DPR may be implemented together with the two mentioned sensors DS, LFP-5580C-337 and LFP-80105C-337, to achieve a workable wavelength range of 5,500 nm to 10,500 nm. If space or mechanics require so, or standard solutions are preferable, a prism DPR and possibly infrared emitter DE may be required for each implemented sensor DS.

The surface characterizing device system SCD comprises a processor DP configured to control the emitter driver DED and sensor driver DSD and to process the output data from the sensor DS into a representation of the absorbance of the surface SU as a function of wavelength, i.e. a spectroscopy measurement ME. In an example configuration, the spectroscopy measurement ME output from the processor DP comprises 250 digital values representing the absorbance at 250 individual wavelengths in the range from 8,000 nm to 10,500 nm, together with a scan ID and a time stamp. In another example configuration, the spectroscopy measurement ME output from the processor DP comprises 250 digital values representing the absorbance at 250 individual wavelengths in the range from 5,500 nm to 8,000 nm, together with a scan ID and a time stamp. In an example configuration having two sensors DS with different wavelength ranges, the spectroscopy measurement ME output from the processor DP may for example comprise 500 digital values representing the absorbance at 500 individual wavelengths in the range from 5,500 nm to 10,500 nm, together with a scan ID and a time stamp. The, e.g., 250 individual absorbance representation values are not necessarily absolute absorbance values, but are typically relative to a common baseline for the individual spectroscopy measurement ME. The processor DP is illustrated as a single block in this example, but may alternatively be distributed among several processing modules. Together with the processor DP, the surface characterizing device system also comprises memory (not shown) to store software instructions such as firmware for the processor DP, predefined configuration parameters and spectroscopy measurements ME.

The surface characterizing device system SCD communicate with remote servers or cloud computing systems as described elsewhere herein, among others to upload or share representations of measurements MER, being representations of spectroscopy measurements ME. The representations of measurements MER may for example be used for training input measurements TIM to train a classification model CM according to the present invention, or be used as input measurement IM for classification of the surface SU comprising a cured coat by means of a trained classification model TCM according to the invention.

The surface characterizing device system SCD may also comprise a user interface DUI, as described elsewhere herein. In an embodiment, a simple user interface DUI simply comprises basic control inputs and status indicators, for example a button for starting a measurement, and a measurement status light signalling when the device is ready for making a measurement, or when a measurement is finished. In an embodiment, a more advanced user interface DUI comprises a display and input buttons, or a touch screen which also function as input means. In a distributed embodiment, a smart device, e.g. a smartphone, laptop or tablet computer, is used for user interface DUI. In an embodiment where the user interface DUI comprises a display, the processor DP may be configured to show a measurement result and more detailed status on the display after a spectroscopy measurement ME is acquired. In an embodiment the surface characterizing device system SCD comprises a trained classification model TCM according to the present invention, and the processor DP is configured to perform a classification of the surface SU comprising a cured coat based on the spectroscopy measurement ME acquired by the surface characterizing device system SCD. In an embodiment, the user interface DUI is configured to allow the operator DO to input a training input label TIL, i.e. a surface characteristic class SCC, to be assigned to the spectroscopy measurement ME in order for the spectroscopy measurement ME to be used to train, test or validate the classification system CS. In an embodiment the user interface DUI is remote controllable, for example when the surface characterizing device system SCD is transported to the measurement location by a drone or robot DR as described above. The remote control communication may use the communication module DCM or a separate remote control module, based on for example Wi-Fi, Bluetooth, BLE, mobile data or other wireless remote control technologies, or be a tethered remote control where the communication is wired, e.g. by a USB or Ethernet connection. In an embodiment, a local smart device SD, e.g. a smartphone, tablet or laptop, connected to or part of the surface characterizing device system SCD for example via the communication module DCM, may also function as an additional user interface to supplement the, possibly quite simple, user interface DUI of the surface characterizing device system SCD.

The surface characterizing device system SCD may comprise further modules (not illustrated) for facilitating good measurements and/or enable further functionality. For example, the surface characterizing device system SCD may comprise one or more pressure sensors and/or accelerometers to monitor the pressure applied by the prism DPR to the surface SU during measurement or to monitor the degree of stability at which the device is held during measurement. The measured pressure or stability information may be fed back to the device operator DO or robot/drone DR during or before measurement to facilitate better circumstances for the measurement, and/or be stored or transmitted together with the spectroscopy measurement ME as metadata providing information about the quality of the measurement, e.g. for a training system or classification system to automatically detect spectroscopy measurements ME that are unsuitable for training or classification, so-called bad scans. For example, the surface characterizing device system SCD may comprise a calibration system to calibrate the ATR system and spectroscopy measurement processing before performing spectroscopy measurements of a surface SU comprising a cured coat. For example, the surface characterizing device system SCD may comprise a camera for recording images in a visible light range, i.e. ‘normal’ pictures of the surface SU comprising a cured coat. The normal images may for example be used to guide the device operator DO or autonomic navigation systems of a drone/robot DR, or they may be used to identify other characteristics of the surface, such as cracks, corrosion, biologic material, etc.

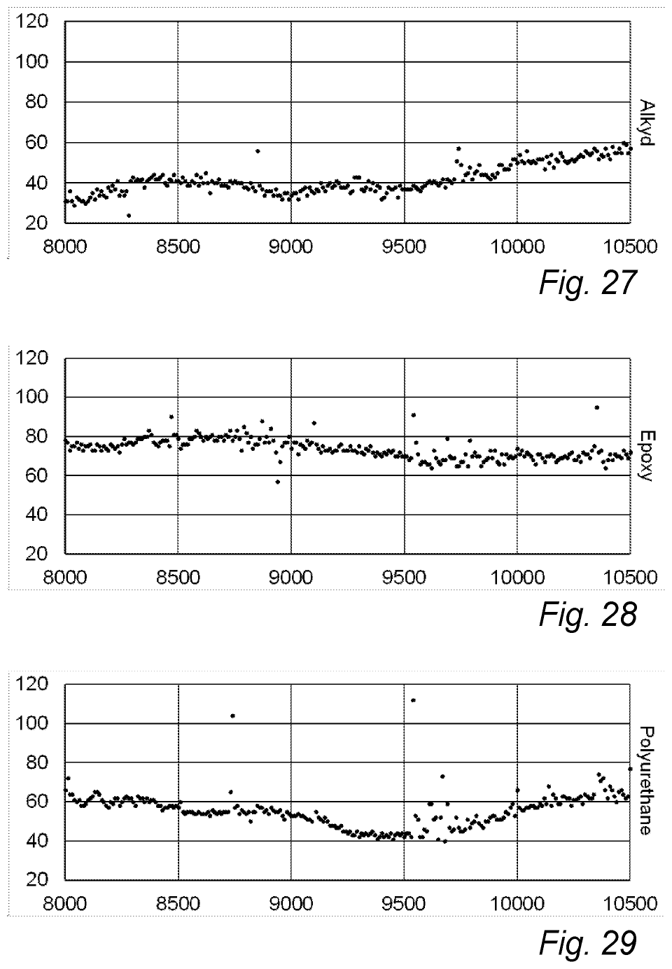

FIGS. 27-29 illustrate examples of spectroscopy measurements ME acquired by the embodiment of a surface characterising device SCD configured to perform ATR-based spectroscopy measurements described above with reference to FIGS. 25-26. All FIGS. 27-29 represent absorbance by a digital value on the vertical axis as a function of wavelength denoted in nanometres on the horizontal axis, and they all have 250 individual values distributed over the wavelength range of 8,000 nm to 10,500 nm. The spectroscopy measurement ME illustrated in FIG. 27 was performed on a surface SU comprising a cured coat which uses alkyd as a binder system, and the spectroscopy measurements ME in FIGS. 28 and 29 were performed on surfaces SU comprising cured coats using epoxy and polyurethane binder systems, respectively, as indicated at the right side of the drawings.

Further examples of spectroscopy measurements ME acquired by the surface characterising device SCD configured to perform ATR-based spectroscopy measurements described above with reference to FIGS. 25-26, are shown here in table form, together with their binder-system class labels, in Table 1 below:

TABLE 1
Labelled spectroscopy measurements
Table 1 - Labelled spectroscopy measurements

| Binder- | | Wave- | Wave- | |
|---|---|---|---|---|
| system | | length | length | |
| class | ID | min. | max. | Measurement values |
| Epoxy | 8ED1FD07- | 8000 | 10500 | [60, 64, 63, 60, 60, 60, 58, 59, 59, 58, 58, 58, 57, 63, 59, |
| | 87ED- | | | 57, 58, 58, 56, 58, 59, 60, 62, 59, 63, 60, 66, 63, 64, 62, |
| | 43E8- | | | 62, 68, 64, 63, 67, 67, 67, 66, 63, 67, 65, 66, 66, 64, 63, |
| | 914A- | | | 62, 71, 63, 65, 60, 63, 63, 62, 62, 67, 60, 65, 64, 65, 63, |
| | 0103AA717C00 | | | 61, 63, 60, 65, 60, 62, 62, 63, 63, 63, 64, 59, 61, 59, 62, |
| | | | | 62, 61, 63, 61, 61, 62, 60, 62, 63, 60, 88, 67, 62, 63, 60, |
| | | | | 60, 60, 60, 57, 64, 67, 64, 59, 60, 61, 59, 59, 60, 64, 62, |
| | | | | 63, 59, 59, 58, 62, 58, 69, 56, 60, 63, 62, 60, 60, 67, 59, |
| | | | | 62, 57, 60, 58, 58, 63, 57, 60, 55, 58, 58, 62, 63, 63, 58, |
| | | | | 61, 62, 57, 56, 57, 62, 56, 73, 59, 59, 58, 55, 60, 56, 60, |
| | | | | 58, 58, 51, 57, 53, 57, 53, 52, 53, 55, 52, 52, 55, 50, 53, |
| | | | | 54, 48, 53, 49, 51, 48, 44, 43, 59, 52, 48, 48, 49, 46, 46, |
| | | | | 44, 47, 48, 45, 44, 46, 41, 46, 43, 45, 44, 46, 46, 41, 46, |
| | | | | 43, 42, 42, 41, 40, 48, 45, 42, 42, 45, 40, 42, 42, 41, 43, |
| | | | | 40, 43, 42, 41, 39, 44, 43, 43, 41, 45, 44, 43, 40, 43, 44, |
| | | | | 45, 41, 39, 42, 40, 43, 43, 44, 44, 44, 43, 40, 44, 40, 46, |
| | | | | 42, 43, 43, 49, 43, 46, 50, 46, 46, 43, 45] |
| Bad | A1939583- | 8000 | 10500 | [98, 104, 106, 99, 101, 104, 101, 101, 103, 100, 102, |
| scan | CD42- | | | 101, 95, 102, 103, 98, 99, 101, 100, 102, 98, 101, 102, |
| | 454B- | | | 100, 100, 98, 101, 102, 104, 100, 101, 107, 98, 96, 104, |
| | A610- | | | 102, 99, 99, 99, 100, 102, 103, 99, 97, 100, 100, 102, |
| | 02577E6C486E | | | 100, 100, 99, 99, 102, 97, 100, 100, 101, 103, 100, |
| | | | | 104, 102, 101, 100, 101, 98, 100, 99, 98, 100, 99, 101, |
| | | | | 103, 100, 100, 98, 99, 102, 101, 101, 100, 99, 98, 99, |
| | | | | 99, 100, 96, 133, 98, 100, 98, 100, 102, 100, 98, 102, |
| | | | | 104, 101, 103, 100, 103, 96, 104, 99, 101, 98, 105, |
| | | | | 100, 100, 98, 102, 102, 104, 98, 96, 101, 106, 104, 101, |
| | | | | 100, 103, 99, 103, 100, 96, 96, 101, 101, 102, 101, |
| | | | | 97, 100, 102, 102, 101, 98, 103, 104, 104, 97, 101, 98, |
| | | | | 97, 96, 104, 102, 99, 101, 100, 100, 103, 103, 100, 100, |
| | | | | 99, 104, 101, 100, 100, 101, 101, 100, 101, 100, 103, |
| | | | | 99, 102, 104, 100, 102, 95, 101, 98, 99, 99, 118, 108, |
| | | | | 102, 101, 111, 92, 103, 108, 103, 117, 103, 106, 105, |
| | | | | 101, 100, 102, 106, 99, 101, 99, 99, 100, 102, 99, |
| | | | | 100, 99, 101, 98, 106, 101, 100, 103, 101, 101, 103, 94, |
| | | | | 102, 100, 102, 97, 100, 98, 97, 101, 98, 95, 98, 110, |
| | | | | 102, 105, 101, 99, 102, 98, 96, 102, 101, 102, 97, 101 |
| | | | | 101, 101, 100, 99, 98, 100, 101, 96, 104, 97, 100, 102, |
| | | | | 101, 102, 97, 108, 99, 100] |
| Alkyd | EDD87403- | 8000 | 10500 | [100, 99, 104, 97, 99, 98, 103, 102, 100, 102, 97, 104, |
| | 9129- | | | 103, 99, 100, 104, 104, 100, 98, 104, 104, 104, 102, 95, |
| | 4705- | | | 84, 96, 87, 96, 60, 99, 101, 95, 101, 101, 102, 99, 102, |
| | BB94- | | | 101, 104, 102, 101, 99, 102, 103, 101, 99, 96, 99, 102, |
| | 0372B3643290 | | | 99, 105, 100, 102, 99, 99, 100, 102, 100, 101, 99, |
| | | | | 106, 100, 111, 100, 90, 99, 99, 100, 96, 101, 102, 101, |
| | | | | 99, 98, 97, 101, 103, 98, 98, 97, 99, 97, 100, 100, 100, |
| | | | | 89, 109, 98, 99, 103, 101, 99, 104, 101, 100, 102, 99, |
| | | | | 99, 101, 97, 97, 99, 100, 99, 102, 102, 101, 97, 100, 98, |
| | | | | 97, 98, 99, 102, 98, 100, 102, 98, 96, 101, 107, 101, |
| | | | | 98, 100, 101, 102, 99, 99, 100, 106, 100, 104, 97, 103, |
| | | | | 99, 99, 99, 100, 103, 92, 83, 90, 97, 106, 99, 101, 105, |
| | | | | 98, 102, 101, 99, 97, 99, 99, 94, 104, 100, 97, 103, 99, |
| | | | | 103, 101, 98, 106, 105, 102, 100, 103, 97, 96, 101, 99, |
| | | | | 102, 106, 99, 102, 92, 101, 101, 102, 97, 104, 104, |
| | | | | 104, 96, 100, 99, 99, 100, 99, 97, 104, 100, 102, 97, 94, |
| | | | | 104, 100, 103, 98, 100, 105, 99, 101, 102, 100, 102, |
| | | | | 103, 99, 102, 101, 102, 100, 103, 99, 99, 101, 101, 96, |
| | | | | 97, 100, 101, 98, 104, 84, 104, 100, 99, 103, 103, 97, |
| | | | | 99, 99, 99, 106, 97, 101, 106, 98, 101, 103, 101, 99, |
| | | | | 101, 100, 97, 105, 97, 102, 100, 102] |
| Bad | 20EE85F3- | 8000 | 10500 | [95, 100, 102, 97, 99, 102, 96, 97, 99, 99, 98, 97, 96, 97, |
| scan | DA57- | | | 103, 94, 98, 98, 96, 100, 97, 100, 99, 98, 98, 96, 97, |
| | 4B70- | | | 100, 97, 98, 99, 97, 99, 99, 99, 98, 100, 97, 96, 98, 99, |
| | 9457- | | | 100, 97, 98, 99, 97, 97, 98, 97, 96, 97, 97, 98, 96, 97, 98, |
| | 038D1A3D5B08 | | | 97, 99, 96, 100, 100, 98, 96, 97, 99, 100, 97, 99, 96, |
| | | | | 101, 100, 99, 96, 99, 98, 102, 98, 99, 97, 98, 97, 95, 93, |
| | | | | 99, 97, 110, 100, 97, 97, 96, 98, 98, 96, 96, 101, 92, 99, |
| | | | | 95, 96, 95, 94, 98, 95, 96, 98, 96, 93, 96, 101, 98, 98, |
| | | | | 97, 95, 100, 102, 99, 94, 94, 103, 95, 101, 97, 95, 97, 97, |
| | | | | 96, 95, 95, 95, 96, 96, 98, 95, 97, 100, 97, 97, 95, 95, |
| | | | | 93, 94, 94, 98, 96, 95, 98, 94, 94, 94, 95, 97, 101, 95, 96, |
| | | | | 98, 96, 97, 92, 100, 98, 96, 96, 96, 97, 99, 98, 97, 96, |
| | | | | 95, 98, 92, 93, 92, 97, 118, 93, 92, 96, 97, 94, 99, 97, 96, |
| | | | | 95, 99, 94, 96, 96, 97, 97, 95, 95, 98, 92, 98, 96, 93, 96, |
| | | | | 94, 95, 98, 95, 95, 92, 99, 97, 95, 98, 91, 102, 91, 93, |
| | | | | 94, 95, 94, 93, 98, 91, 91, 95, 97, 99, 98, 95, 91, 95, 94, |
| | | | | 95, 95, 94, 97, 89, 97, 95, 93, 93, 96, 88, 92, 94, 96, 98, |
| | | | | 93, 94, 97, 97, 102, 91, 98, 96, 94] |
| Bad | BF1D1A7E- | 8000 | 10500 | [93, 85, 85, 84, 85, 82, 85, 85, 85, 90, 84, 86, 86, 86, 88, |
| scan | 6183- | | | 84, 83, 85, 86, 85, 87, 87, 90, 89, 84, 88, 87, 87, 84, 87, |
| | 44AB- | | | 85, 80, 88, 81, 85, 85, 89, 87, 86, 74, 84, 87, 78, 85, 88, |
| | BFE0- | | | 87, 86, 85, 86, 83, 88, 84, 88, 83, 86, 87, 84, 84, 86, 85, |
| | 04F777222CAB | | | 87, 90, 87, 86, 86, 82, 83, 86, 84, 85, 85, 83, 82, 87, 82, |
| | | | | 90, 87, 85, 83, 80, 85, 86, 84, 85, 85, 117, 86, 83, 85, |
| | | | | 87, 81, 84, 85, 86, 84, 80, 81, 81, 88, 80, 78, 79, 81, 83, |
| | | | | 84, 85, 80, 79, 88, 81, 85, 82, 82, 81, 85, 80, 84, 83, 84, |
| | | | | 83, 80, 86, 82, 84, 82, 87, 89, 81, 82, 86, 80, 80, 86, 85, |
| | | | | 85, 79, 84, 81, 83, 82, 86, 83, 80, 81, 82, 84, 82, 82, 83, |
| | | | | 84, 83, 83, 82, 81, 86, 81, 78, 78, 81, 84, 82, 83, 84, 84, |
| | | | | 81, 77, 83, 83, 82, 86, 81, 81, 78, 91, 124, 85, 83, 84, 84, |
| | | | | 85, 86, 84, 88, 94, 82, 84, 85, 87, 84, 82, 88, 85, 84, 84, |
| | | | | 85, 81, 82, 86, 82, 85, 92, 85, 84, 84, 88, 86, 85, 89, 84, |
| | | | | 85, 85, 88, 87, 86, 87, 84, 83, 87, 87, 87, 83, 86, 83, 84, |
| | | | | 83, 90, 89, 84, 84, 83, 83, 83, 88, 81, 84, 88, 88, 83, 93, |
| | | | | 87, 82, 91, 89, 87, 82, 88, 91, 87, 86, 88, 86] |
| Alkyd | 471C325F- | 8000 | 10500 | [31, 31, 36, 31, 29, 33, 32, 31, 31, 30, 31, 33, 35, 32, 37, |
| | E72B- | | | 34, 36, 34, 33, 38, 36, 39, 37, 41, 34, 36, 34, 36, 24, 41, |
| | 4BDA- | | | 43, 41, 42, 41, 42, 38, 41, 42, 43, 44, 42, 42, 43, 44, 40, |
| | 8B0B- | | | 39, 42, 41, 44, 41, 41, 40, 43, 41, 39, 39, 41, 40, 44, 39, |
| | 080A4EF748A6 | | | 43, 40, 45, 40, 35, 41, 40, 40, 42, 39, 39, 39, 38, 41, 40, |
| | | | | 41, 40, 40, 40, 38, 38, 37, 40, 38, 36, 56, 39, 37, 34, 36, |
| | | | | 36, 36, 34, 37, 34, 34, 32, 35, 35, 32, 34, 35, 35, 32, 36, |
| | | | | 37, 38, 34, 38, 36, 35, 37, 36, 40, 38, 36, 39, 40, 38, 39, |
| | | | | 41, 40, 38, 38, 38, 39, 35, 36, 43, 43, 43, 37, 36, 38, 41, |
| | | | | 37, 40, 36, 39, 38, 32, 33, 35, 38, 37, 37, 38, 33, 39, 37, |
| | | | | 37, 37, 37, 37, 39, 38, 37, 36, 38, 39, 40, 41, 40, 41, 40, |
| | | | | 39, 40, 42, 38, 42, 41, 40, 42, 51, 57, 49, 41, 44, 45, 48, |
| | | | | 42, 45, 46, 49, 44, 44, 44, 43, 42, 44, 43, 45, 49, 49, 47, |
| | | | | 47, 47, 49, 52, 52, 50, 54, 51, 50, 56, 51, 50, 51, 50, 52, |
| | | | | 52, 52, 47, 53, 50, 54, 48, 52, 51, 55, 53, 51, 50, 51, 52, |
| | | | | 51, 52, 53, 54, 53, 56, 55, 54, 55, 57, 56, 52, 54, 53, 57, |
| | | | | 54, 52, 58, 55, 55, 57, 55, 60, 59, 55, 57] |
| Epoxy | 71012548- | 8000 | 10500 | [99, 102, 99, 102, 101, 100, 100, 98, 108, 99, 101, |
| | 985E- | | | 102, 102, 100, 99, 100, 100, 100, 99, 102, 97, 95, 102, |
| | 4C21- | | | 102, 103, 100, 99, 101, 102, 98, 98, 99, 106, 101, 98, |
| | A033- | | | 102, 103, 102, 101, 99, 97, 102, 98, 100, 97, 102, 100, |
| | 0B2D1BAC7F5D | | | 102, 98, 105, 97, 96, 102, 97, 100, 99, 102, 99, 98, |
| | | | | 100, 98, 101, 100, 97, 102, 101, 103, 100, 94, 98, 101, |
| | | | | 97, 99, 101, 99, 102, 100, 100, 96, 102, 100, 99, 100, |
| | | | | 97, 105, 177, 111, 98, 97, 102, 98, 63, 103, 100, 100, |
| | | | | 101, 102, 96, 97, 96, 97, 98, 100, 97, 101, 101, 104, 98, |
| | | | | 99, 101, 98, 95, 101, 101, 102, 98, 97, 96, 100, 101, |
| | | | | 96, 101, 105, 100, 99, 103, 93, 99, 94, 100, 96, 98, 100, |
| | | | | 102, 99, 98, 99, 99, 100, 103, 103, 104, 102, 104, 96, |
| | | | | 103, 99, 98, 98, 99, 99, 102, 101, 100, 101, 98, 98, |
| | | | | 101, 98, 102, 103, 98, 107, 96, 100, 106, 100, 99, 100, |
| | | | | 99, 97, 98, 93, 97, 94, 97, 102, 100, 98, 101, 98, 100, |
| | | | | 101, 105, 96, 99, 102, 102, 99, 97, 100, 99, 100, 102, 98, |
| | | | | 97, 101, 102, 101, 101, 102, 103, 106, 98, 104, 105, |
| | | | | 104, 100, 95, 94, 97, 94, 93, 98, 99, 99, 103, 100, 100, |
| | | | | 95, 94, 96, 98, 99, 106, 99, 99, 101, 102, 94, 104, 102, |
| | | | | 97, 99, 101, 103, 98, 95, 102, 106, 98, 93, 100, 102, |
| | | | | 100, 100, 101, 99, 96, 103, 99] |
| Polyurethane | AA4E8448- | 8000 | 10500 | [75, 72, 75, 72, 73, 75, 76, 74, 77, 75, 76, 74, 78, 75, 72, |
| | 4421- | | | 76, 76, 75, 73, 77, 75, 76, 75, 72, 63, 72, 64, 72, 43, 73, |
| | 434E- | | | 77, 70, 72, 74, 76, 71, 74, 73, 75, 74, 74, 73, 72, 74, 71, |
| | 9AAC- | | | 70, 72, 71, 73, 72, 75, 73, 71, 72, 71, 72, 72, 70, 72, 70, |
| | 0D74731750E6 | | | 74, 72, 80, 71, 65, 71, 72, 71, 68, 72, 70, 74, 71, 74, 71, |
| | | | | 74, 70, 71, 71, 94, 67, 68, 72, 75, 71, 65, 75, 72, 73, 75, |
| | | | | 74, 71, 73, 73, 72, 72, 74, 69, 68, 68, 69, 71, 70, 68, 74, |
| | | | | 70, 72, 67, 72, 70, 68, 70, 68, 71, 70, 72, 72, 66, 62, 74, |
| | | | | 71, 68, 70, 67, 69, 72, 66, 67, 69, 68, 65, 67, 65, 68, 67, |
| | | | | 68, 67, 69, 70, 68, 60, 62, 64, 72, 67, 67, 67, 66, 71, 67, |
| | | | | 67, 65, 67, 67, 64, 71, 66, 65, 69, 68, 66, 68, 69, 69, 67, |
| | | | | 67, 66, 66, 62, 59, 67, 62, 67, 72, 100, 70, 59, 66, 65, 68, |
| | | | | 63, 64, 65, 66, 62, 61, 61, 63, 64, 67, 63, 66, 65, 62, 61, |
| | | | | 63, 68, 66, 67, 60, 66, 62, 66, 62, 62, 66, 69, 68, 64, 64, |
| | | | | 66, 65, 61, 66, 63, 67, 68, 67, 64, 65, 67, 65, 65, 65, 66, |
| | | | | 64, 63, 65, 66, 66, 63, 70, 67, 67, 72, 72, 70, 70, 64, 71, |
| | | | | 68, 68, 67, 70, 72, 71, 72, 68, 68, 73, 70] |
| Polyurethane | 2C62F5A6- | 8000 | 10500 | [74, 73, 73, 71, 67, 70, 73, 74, 74, 74, 74, 74, 74, 66, 72, |
| | 2092- | | | 72, 77, 72, 71, 73, 70, 73, 76, 70, 61, 69, 61, 69, 43, 72, |
| | 449E- | | | 76, 71, 74, 70, 71, 68, 70, 71, 74, 72, 70, 72, 72, 71, 66, |
| | 8E4C- | | | 68, 68, 68, 70, 68, 72, 67, 67, 66, 68, 70, 68, 64, 68, 67, |
| | 0E39CDD4A87D | | | 67, 72, 73, 64, 59, 63, 66, 67, 67, 64, 61, 65, 63, 67, 65, |
| | | | | 65, 67, 64, 66, 65, 66, 67, 68, 67, 66, 85, 69, 64, 67, 67, |
| | | | | 65, 66, 70, 67, 68, 69, 66, 67, 65, 63, 64, 66, 66, 63, 68, |
| | | | | 67, 67, 63, 68, 64, 67, 65, 69, 64, 63, 62, 70, 66, 69, 63, |
| | | | | 72, 65, 62, 61, 63, 64, 64, 62, 62, 69, 65, 63, 63, 63, 62, |
| | | | | 64, 63, 64, 65, 61, 54, 62, 59, 65, 63, 67, 66, 64, 64, 66, |
| | | | | 67, 66, 66, 64, 64, 67, 66, 63, 66, 66, 68, 67, 66, 72, 68, |
| | | | | 68, 66, 67, 63, 67, 69, 65, 68, 66, 84, 67, 64, 67, 67, 65, |
| | | | | 66, 68, 72, 72, 68, 69, 71, 69, 68, 67, 68, 65, 67, 69, 66, |
| | | | | 67, 74, 66, 74, 70, 77, 66, 73, 67, 69, 72, 70, 72, 72, 76, |
| | | | | 71, 71, 71, 73, 73, 72, 74, 71, 71, 75, 69, 73, 69, 74, 72, |
| | | | | 75, 76, 74, 73, 74, 72, 77, 74, 77, 77, 74, 71, 77, 73, 78, |
| | | | | 77, 72, 77, 73, 77, 71, 79, 75, 74, 77, 78] |
| Epoxy | 1E366D23- | 8000 | 10500 | [92, 97, 112, 103, 94, 119, 97, 89, 100, 92, 92, 86, 96, |
| | C041- | | | 91, 94, 95, 90, 95, 87, 97, 88, 92, 90, 95, 86, 95, 92, 93, |
| | 4266- | | | 96, 91, 130, 93, 91, 88, 90, 88, 92, 94, 91, 92, 90, 90, 89, |
| | 9617- | | | 93, 88, 96, 89, 92, 90, 93, 88, 91, 91, 97, 95, 94, 91, 91, |
| | 0F1AA02A8D00 | | | 92, 88, 91, 90, 91, 89, 95, 90, 93, 95, 96, 88, 91, 93, 93, |
| | | | | 93, 91, 92, 89, 91, 93, 92, 91, 92, 93, 93, 94, 172, 95, |
| | | | | 95, 93, 97, 100, 95, 96, 101, 97, 101, 88, 96, 91, 97, 88, |
| | | | | 95, 89, 96, 84, 95, 89, 94, 93, 94, 91, 88, 95, 91, 94, 92, |
| | | | | 99, 87, 98, 88, 99, 90, 88, 89, 89, 89, 90, 89, 89, 90, 86, |
| | | | | 87, 89, 90, 86, 89, 88, 89, 90, 89, 91, 87, 87, 87, 86, 91, |
| | | | | 90, 88, 88, 91, 87, 89, 89, 87, 88, 89, 87, 88, 85, 83, 89, |
| | | | | 87, 89, 84, 87, 89, 82, 91, 87, 86, 85, 83, 86, 99, 156, |
| | | | | 85, 93, 85, 92, 85, 85, 88, 91, 87, 95, 90, 86, 87, 87, 88, |
| | | | | 89, 87, 84, 85, 89, 88, 89, 88, 89, 94, 89, 87, 90, 86, 94, |
| | | | | 87, 88, 85, 90, 100, 87, 90, 82, 88, 87, 88, 94, 92, 92, 89, |
| | | | | 92, 88, 89, 96, 90, 91, 88, 92, 86, 91, 88, 88, 88, 92, 86, |
| | | | | 90, 90, 91, 90, 91, 92, 88, 91, 89, 93, 91, 96, 90, 91, 91, |
| | | | | 93] |
| Epoxy | F9D284DA- | 8000 | 10500 | [95, 104, 100, 104, 102, 100, 101, 100, 100, 101, 102, |
| | 410D- | | | 99, 102, 100, 101, 98, 101, 101, 99, 101, 101, 99, 104, |
| | 4A0B- | | | 102, 101, 100, 97, 100, 101, 98, 104, 100, 102, 100, |
| | 9710- | | | 100, 101, 99, 98, 102, 100, 99, 101, 102, 99, 96, 100, |
| | 0FEB7E2C5632 | | | 102, 101, 98, 100, 97, 98, 101, 98, 99, 100, 101, 100, |
| | | | | 100, 102, 100, 103, 101, 99, 103, 97, 102, 101, 100, 97, |
| | | | | 100, 98, 98, 100, 102, 100, 99, 98, 98, 98, 99, 99, 100, |
| | | | | 99, 103, 114, 108, 103, 99, 101, 100, 63, 104, 99, 102, |
| | | | | 100, 101, 96, 100, 95, 100, 100, 104, 99, 102, 101, |
| | | | | 96, 102, 101, 98, 103, 98, 103, 101, 101, 99, 98, 98, |
| | | | | 101, 99, 104, 98, 103, 100, 102, 103, 95, 98, 98, 99, 97, |
| | | | | 100, 99, 104, 97, 99, 95, 97, 98, 104, 102, 103, 101, |
| | | | | 105, 102, 104, 99, 100, 98, 103, 103, 100, 105, 101, |
| | | | | 102, 102, 96, 101, 101, 100, 102, 101, 104, 95, 96, 102, |
| | | | | 100, 99, 99, 97, 97, 99, 99, 101, 106, 101, 102, 99, 101, |
| | | | | 99, 100, 99, 103, 100, 102, 100, 102, 99, 101, 96, 103, |
| | | | | 99, 104, 100, 103, 98, 101, 97, 102, 100, 100, 102, |
| | | | | 104, 101, 108, 102, 103, 100, 99, 100, 97, 95, 97, |
| | | | | 100, 101, 100, 100, 100, 102, 97, 103, 99, 103, 99, 101, |
| | | | | 100, 103, 103, 103, 97, 103, 101, 102, 99, 101, 102, |
| | | | | 104, 102, 98, 102, 100, 96, 103, 99, 98, 100, 100, 100, |
| | | | | 94, 107, 100] |
| Bad | 039F007B- | 8000 | 10500 | [75, 77, 77, 74, 75, 78, 75, 74, 74, 72, 73, 72, 74, 74, 76, |
| scan | FC90- | | | 72, 74, 75, 75, 74, 74, 73, 78, 75, 76, 76, 75, 76, 76, 74, |
| | 4096- | | | 76, 73, 79, 77, 78, 78, 79, 77, 77, 80, 80, 78, 81, 76, 78, |
| | B857- | | | 78, 77, 78, 77, 80, 76, 79, 76, 78, 78, 76, 79, 77, 78, 78, |
| | 1216BAD28002 | | | 79, 78, 78, 80, 80, 85, 77, 79, 78, 84, 82, 79, 78, 78, 77, |
| | | | | 80, 77, 78, 79, 78, 75, 77, 76, 77, 81, 103, 79, 78, 79, |
| | | | | 79, 76, 74, 75, 77, 79, 81, 80, 76, 78, 77, 75, 83, 77, 80, |
| | | | | 77, 76, 72, 76, 75, 78, 76, 90, 74, 78, 80, 76, 76, 78, 75, |
| | | | | 81, 75, 77, 75, 79, 77, 77, 76, 76, 72, 76, 76, 74, 72, 74, |
| | | | | 76, 78, 78, 70, 76, 73, 71, 76, 76, 75, 75, 79, 72, 74, 76, |
| | | | | 77, 73, 76, 72, 75, 74, 75, 72, 70, 74, 77, 75, 73, 72, 74, |
| | | | | 72, 72, 73, 74, 69, 71, 68, 68, 71, 80, 96, 76, 68, 69, 72, |
| | | | | 72, 70, 70, 76, 71, 73, 72, 73, 83, 73, 73, 71, 70, 68, 70, |
| | | | | 68, 69, 68, 78, 73, 70, 71, 71, 67, 69, 70, 72, 70, 72, 73, |
| | | | | 79, 69, 69, 74, 72, 66, 69, 72, 70, 70, 69, 71, 73, 72, 68, |
| | | | | 67, 68, 71, 67, 74, 71, 70, 66, 72, 69, 69, 70, 73, 71, 71, |
| | | | | 69, 71, 71, 74, 73, 73, 71, 72, 74, 73, 76, 71] |
| Alkyd | 1C156B06- | 8000 | 10500 | [33, 30, 29, 27, 29, 36, 32, 32, 32, 33, 33, 32, 34, 35, 36, |
| | 9519- | | | 35, 32, 35, 35, 36, 37, 35, 41, 38, 39, 40, 38, 39, 38, 41, |
| | 4328- | | | 41, 49, 43, 40, 40, 45, 42, 44, 42, 38, 41, 46, 43, 42, 43, |
| | A56E- | | | 44, 42, 44, 43, 43, 45, 41, 43, 42, 43, 43, 41, 44, 42, 42, |
| | 1636F1078819 | | | 43, 42, 42, 41, 42, 38, 39, 41, 42, 40, 41, 39, 39, 40, 42, |
| | | | | 43, 41, 41, 39, 41, 39, 39, 39, 36, 37, 67, 38, 36, 35, 38, |
| | | | | 37, 35, 36, 37, 34, 40, 33, 37, 35, 33, 39, 32, 36, 33, 35, |
| | | | | 35, 33, 36, 41, 38, 36, 37, 39, 38, 39, 42, 38, 38, 39, 41, |
| | | | | 39, 41, 42, 37, 39, 40, 40, 42, 38, 42, 40, 42, 39, 40, 37, |
| | | | | 39, 40, 37, 37, 39, 36, 41, 36, 37, 35, 38, 39, 39, 36, 37, |
| | | | | 37, 40, 37, 39, 40, 36, 37, 38, 39, 40, 39, 40, 38, 42, 38, |
| | | | | 41, 38, 38, 39, 42, 43, 40, 38, 45, 55, 46, 44, 41, 39, 43, |
| | | | | 44, 44, 42, 44, 44, 44, 49, 51, 51, 44, 48, 48, 47, 50, 47, |
| | | | | 46, 47, 49, 51, 45, 50, 49, 48, 53, 52, 49, 53, 50, 47, 53, |
| | | | | 56, 51, 52, 48, 54, 51, 51, 51, 55, 56, 54, 53, 49, 53, 52, |
| | | | | 56, 58, 54, 56, 54, 52, 58, 56, 55, 53, 51, 56, 55, 57, 55, |
| | | | | 57, 59, 59, 58, 54, 58, 54, 56, 60, 56, 57] |
| Epoxy | 78AAA17D- | 8000 | 10500 | [103, 103, 96, 104, 99, 102, 102, 97, 103, 102, 99, |
| | EEDF- | | | 101, 101, 99, 100, 99, 98, 99, 98, 103, 99, 100, 105, 103, |
| | 4AE7- | | | 106, 99, 98, 102, 99, 100, 100, 99, 103, 107, 100, 100, |
| | 88F0- | | | 98, 103, 101, 99, 95, 102, 102, 99, 97, 101, 101, 101, |
| | 1795F13B2899 | | | 99, 100, 97, 99, 100, 101, 100, 100, 101, 97, 100, 101, |
| | | | | 105, 102, 102, 98, 102, 101, 104, 103, 101, 98, 95, 100, |
| | | | | 101, 101, 101, 100, 98, 99, 98, 101, 98, 100, 99, 98, |
| | | | | 102, 118, 104, 102, 101, 99, 99, 63, 102, 99, 99, 103, |
| | | | | 103, 99, 105, 93, 98, 96, 100, 97, 102, 99, 98, 102, 101, |
| | | | | 101, 102, 98, 104, 103, 99, 98, 98, 100, 100, 103, |
| | | | | 98, 99, 107, 103, 99, 102, 94, 97, 98, 104, 100, 104, |
| | | | | 105, 100, 100, 102, 99, 98, 100, 106, 101, 102, 101, 106, |
| | | | | 98, 100, 101, 97, 101, 99, 102, 101, 103, 100, 103, |
| | | | | 97, 99, 99, 100, 103, 102, 96, 105, 96, 99, 104, 99, 100, |
| | | | | 99, 96, 99, 99, 98, 99, 120, 95, 98, 101, 102, 101, 102, |
| | | | | 99, 107, 104, 99, 100, 100, 98, 100, 98, 96, 97, 102, |
| | | | | 98, 101, 98, 98, 98, 101, 99, 96, 99, 101, 100, 100, 108, |
| | | | | 102, 94, 100, 96, 104, 97, 94, 100, 100, 100, 103, 99, |
| | | | | 101, 96, 102, 95, 99, 101, 102, 101, 99, 97, 100, 99, |
| | | | | 99, 100, 102, 100, 102, 104, 101, 100, 103, 107, 104, |
| | | | | 99, 103, 95, 97, 97, 96, 104, 98, 104, 101] |
| Epoxy | 8C97536D- | 8000 | 10500 | [92, 93, 90, 97, 92, 94, 92, 93, 92, 89, 91, 90, 92, 91, 94, |
| | DC62- | | | 92, 93, 93, 93, 93, 92, 91, 92, 92, 93, 93, 90, 92, 94, 92, |
| | 4416- | | | 93, 91, 95, 93, 90, 93, 92, 93, 94, 91, 91, 95, 91, 95, 90, |
| | A144- | | | 93, 95, 94, 91, 94, 90, 91, 96, 92, 91, 94, 94, 92, 92, 93, |
| | 2026D8FC595F | | | 93, 95, 94, 92, 95, 94, 96, 95, 94, 93, 94, 91, 93, 94, 91, |
| | | | | 93, 94, 94, 90, 93, 89, 93, 93, 90, 97, 102, 97, 97, 92, 89, |
| | | | | 93, 58, 92, 93, 92, 95, 94, 89, 91, 88, 92, 94, 94, 90, 93, |
| | | | | 91, 92, 93, 89, 90, 95, 88, 95, 96, 91, 92, 89, 92, 94, 91, |
| | | | | 98, 87, 94, 90, 92, 93, 89, 91, 89, 92, 87, 90, 91, 93, 94, |
| | | | | 91, 90, 89, 92, 95, 94, 97, 94, 95, 89, 96, 94, 90, 89, 96, |
| | | | | 92, 92, 93, 90, 94, 90, 90, 89, 92, 93, 99, 91, 96, 88, 90, |
| | | | | 93, 87, 90, 91, 91, 88, 90, 92, 101, 115, 89, 90, 89, 93, |
| | | | | 88, 93, 87, 90, 95, 90, 89, 91, 94, 89, 90, 93, 87, 91, 97, |
| | | | | 91, 88, 89, 91, 90, 89, 93, 93, 91, 88, 91, 93, 92, 87, 93, |
| | | | | 88, 93, 87, 86, 90, 90, 92, 91, 88, 87, 89, 91, 87, 90, 85, |
| | | | | 89, 93, 92, 90, 92, 87, 92, 89, 85, 91, 93, 91, 89, 87, 91, |
| | | | | 92, 96, 91, 92, 89, 88, 93, 88, 91, 91, 91, 91] |
| Polyurethane | 6628DEDA- | 8000 | 10500 | [63, 62, 56, 65, 62, 62, 63, 62, 61, 60, 62, 62, 63, 59, 62, |
| | 805D- | | | 61, 63, 63, 58, 61, 57, 59, 59, 60, 61, 58, 60, 62, 60, 57, |
| | 4B91- | | | 63, 61, 58, 60, 60, 60, 58, 60, 58, 58, 56, 60, 56, 57, 52, |
| | B56A- | | | 55, 55, 55, 54, 54, 54, 51, 53, 54, 53, 54, 52, 51, 52, 51, |
| | 206A7D65312C | | | 51, 52, 52, 50, 51, 50, 50, 51, 52, 50, 51, 46, 50, 51, 50, |
| | | | | 52, 52, 49, 49, 50, 50, 51, 52, 52, 51, 62, 56, 51, 50, 50, |
| | | | | 51, 34, 57, 49, 51, 49, 52, 50, 51, 49, 49, 50, 53, 49, 51, |
| | | | | 50, 51, 50, 51, 48, 48, 50, 51, 53, 48, 50, 49, 48, 49, 51, |
| | | | | 49, 46, 50, 47, 49, 48, 45, 47, 42, 47, 48, 47, 48, 47, 47, |
| | | | | 47, 50, 46, 46, 48, 44, 47, 48, 50, 44, 48, 45, 45, 46, 46, |
| | | | | 47, 49, 49, 49, 49, 49, 45, 51, 51, 53, 53, 49, 55, 53, 53, |
| | | | | 54, 50, 52, 51, 55, 50, 50, 52, 53, 58, 50, 48, 53, 51, 54, |
| | | | | 50, 54, 53, 52, 57, 53, 55, 54, 48, 53, 52, 54, 54, 54, 54, |
| | | | | 53, 53, 55, 57, 55, 51, 55, 60, 53, 58, 54, 59, 56, 55, 54, |
| | | | | 58, 56, 61, 59, 59, 58, 60, 62, 58, 57, 62, 57, 57, 60, 59, |
| | | | | 62, 60, 56, 62, 62, 60, 57, 62, 60, 64, 60, 61, 60, 61, 59, |
| | | | | 60, 63, 63, 63, 62, 63, 60, 65, 59, 63, 60] |
| Epoxy | 0576E7E4- | 8000 | 10500 | [69, 71, 71, 67, 69, 71, 67, 65, 63, 66, 63, 65, 63, 66, 65, |
| | 35E0- | | | 62, 64, 66, 63, 66, 63, 64, 66, 67, 65, 64, 66, 66, 66, 68, |
| | 4ECC- | | | 72, 75, 70, 69, 71, 69, 71, 70, 69, 72, 73, 70, 70, 70, 70, |
| | B650- | | | 68, 70, 69, 70, 69, 70, 69, 63, 68, 68, 69, 69, 71, 70, 69, |
| | 254877B11B97 | | | 71, 71, 71, 69, 73, 71, 71, 74, 69, 71, 72, 70, 71, 70, 72, |
| | | | | 71, 71, 70, 71, 70, 69, 70, 71, 68, 70, 97, 72, 71, 67, 68, |
| | | | | 68, 69, 67, 70, 68, 65, 69, 64, 70, 66, 65, 66, 66, 65, 63, |
| | | | | 64, 60, 61, 62, 65, 65, 62, 60, 63, 66, 64, 63, 59, 61, 61, |
| | | | | 59, 62, 58, 60, 64, 57, 62, 60, 60, 58, 58, 59, 59, 57, 60, |
| | | | | 61, 62, 60, 62, 58, 57, 57, 59, 60, 60, 63, 59, 60, 62, 61, |
| | | | | 57, 58, 55, 61, 64, 58, 58, 59, 58, 60, 58, 58, 56, 55, 56, |
| | | | | 56, 56, 55, 56, 57, 53, 50, 53, 59, 60, 65, 62, 56, 55, 57, |
| | | | | 53, 56, 55, 51, 53, 54, 54, 53, 53, 56, 52, 51, 51, 52, 57, |
| | | | | 54, 54, 57, 57, 55, 55, 58, 56, 52, 53, 55, 59, 55, 53, 59, |
| | | | | 53, 56, 57, 55, 57, 57, 56, 59, 53, 59, 63, 57, 55, 60, 57, |
| | | | | 62, 56, 58, 58, 61, 57, 58, 58, 59, 58, 61, 58, 56, 63, 59, |
| | | | | 60, 62, 58, 60, 59, 61, 61, 60, 60, 61, 61] |
| Epoxy | C41F1DFD- | 8000 | 10500 | [97, 98, 94, 98, 107, 97, 99, 95, 99, 96, 95, 96, 99, 95, |
| | 546A- | | | 97, 95, 97, 96, 92, 97, 93, 93, 99, 100, 96, 96, 94, 97, 96, |
| | 4E4B- | | | 99, 104, 92, 98, 97, 94, 95, 97, 97, 97, 97, 94, 97, 96, |
| | A181- | | | 96, 93, 95, 92, 98, 93, 97, 93, 95, 98, 95, 95, 95, 97, 95, |
| | 26500D301C08 | | | 95, 97, 97, 98, 95, 93, 99, 97, 93, 98, 95, 94, 94, 94, 93, |
| | | | | 97, 98, 96, 94, 96, 94, 95, 95, 95, 98, 94, 100, 123, 96, |
| | | | | 95, 96, 94, 95, 61, 100, 98, 98, 99, 96, 90, 93, 92, 94, 98, |
| | | | | 96, 96, 94, 92, 95, 94, 98, 94, 97, 94, 95, 95, 95, 93, 95, |
| | | | | 96, 100, 95, 95, 96, 100, 93, 97, 99, 90, 95, 93, 93, 92, |
| | | | | 96, 94, 96, 94, 93, 95, 94, 94, 101, 97, 97, 98, 99, 93, |
| | | | | 97, 97, 91, 98, 96, 97, 94, 96, 92, 94, 95, 95, 96, 96, 92, |
| | | | | 96, 89, 99, 92, 91, 97, 93, 96, 90, 90, 89, 93, 88, 98, 105, |
| | | | | 90, 92, 94, 91, 96, 92, 92, 94, 100, 93, 90, 93, 91, 91, |
| | | | | 88, 94, 92, 97, 94, 94, 88, 92, 96, 94, 92, 91, 87, 91, 93, |
| | | | | 92, 94, 96, 91, 93, 92, 99, 87, 91, 93, 90, 94, 95, 91, 94, |
| | | | | 91, 91, 88, 94, 97, 91, 98, 100, 95, 92, 94, 106, 98, 94, |
| | | | | 93, 96, 98, 91, 93, 94, 93, 97, 93, 96, 90, 93, 96, 93, 95, |
| | | | | 96, 95, 95] |
| Alkyd | E4E91B9F- | 8000 | 10500 | [99, 97, 100, 95, 100, 99, 103, 98, 100, 101, 101, 103, |
| | 7437- | | | 103, 100, 99, 100, 102, 99, 97, 101, 101, 104, 102, 97, |
| | 4824- | | | 83, 94, 84, 96, 59, 97, 101, 97, 100, 100, 102, 98, 99, |
| | B23B- | | | 100, 103, 97, 99, 101, 100, 101, 96, 99, 99, 99, 104, |
| | 27313F3BA7BE | | | 98, 105, 98, 99, 100, 98, 99, 101, 99, 99, 97, 104, 97, |
| | | | | 111, 98, 90, 99, 101, 96, 95, 101, 98, 100, 99, 103, 97, |
| | | | | 100, 103, 99, 100, 97, 97, 97, 101, 101, 97, 109, 99, 99, |
| | | | | 94, 101, 100, 100, 106, 100, 97, 101, 101, 99, 98, 97, |
| | | | | 97, 100, 97, 97, 101, 102, 103, 97, 102, 100, 100, 96, |
| | | | | 102, 100, 99, 97, 99, 94, 97, 102, 107, 100, 100, 99, |
| | | | | 103, 102, 97, 97, 98, 102, 102, 100, 95, 98, 101, 99, 98, |
| | | | | 99, 103, 94, 85, 89, 93, 101, 99, 96, 102, 97, 100, 101, |
| | | | | 97, 100, 98, 99, 98, 101, 100, 93, 101, 98, 100, 104, |
| | | | | 102, 101, 101, 100, 99, 102, 100, 104, 99, 102, 103, 99, |
| | | | | 92, 96, 93, 98, 97, 98, 100, 102, 101, 102, 98, 103, 97, |
| | | | | 99, 100, 100, 97, 99, 97, 100, 100, 94, 103, 101, 98, |
| | | | | 94, 101, 101, 104, 96, 101, 102, 102, 101, 104, 99, 103, |
| | | | | 102, 98, 101, 99, 102, 100, 99, 99, 98, 101, 98, 99, 97, |
| | | | | 98, 98, 99, 97, 101, 99, 101, 102, 96, 104, 107, 101, |
| | | | | 98, 101, 99, 107, 100, 96, 95, 100, 106, 101, 104, 99, |
| | | | | 100, 104, 102] |
| Polyurethane | BE848406- | 8000 | 10500 | [57, 57, 61, 58, 57, 55, 57, 56, 61, 58, 57, 58, 57, 55, 56, |
| | 5249- | | | 55, 55, 56, 54, 55, 55, 56, 56, 57, 47, 52, 49, 53, 32, 53, |
| | 4036- | | | 57, 53, 56, 56, 54, 55, 53, 57, 56, 56, 54, 53, 52, 53, 49, |
| | 9743- | | | 51, 52, 49, 49, 49, 53, 49, 50, 48, 45, 44, 49, 46, 45, 46, |
| | 2E9F8062A00C | | | 48, 43, 50, 44, 41, 44, 45, 44, 41, 43, 44, 45, 42, 46, 46, |
| | | | | 44, 50, 43, 45, 45, 45, 45, 45, 47, 43, 58, 49, 44, 44, 45, |
| | | | | 48, 45, 50, 45, 44, 47, 47, 46, 46, 43, 45, 49, 44, 44, 42, |
| | | | | 48, 45, 44, 45, 47, 47, 48, 41, 44, 44, 46, 43, 45, 44, 45, |
| | | | | 45, 44, 42, 40, 42, 44, 42, 41, 44, 43, 44, 42, 42, 41, 42, |
| | | | | 43, 44, 42, 42, 41, 36, 38, 42, 42, 45, 42, 42, 43, 43, 43, |
| | | | | 44, 41, 43, 47, 45, 43, 44, 41, 47, 48, 46, 45, 47, 51, 48, |
| | | | | 48, 56, 47, 47, 46, 47, 48, 49, 48, 44, 47, 43, 46, 48, 47, |
| | | | | 47, 48, 51, 49, 45, 47, 48, 50, 47, 46, 49, 48, 47, 48, 47, |
| | | | | 49, 53, 47, 51, 55, 50, 50, 54, 54, 48, 53, 52, 51, 53, 57, |
| | | | | 55, 51, 52, 53, 52, 56, 55, 54, 52, 49, 52, 58, 50, 54, 53, |
| | | | | 56, 55, 57, 58, 55, 54, 58, 62, 62, 61, 62, 58, 61, 55, 55, |
| | | | | 59, 57, 56, 58, 57, 62, 60, 59, 58, 58, 59] |
| Epoxy | C8B477C3- | 8000 | 10500 | [78, 77, 73, 75, 75, 77, 74, 76, 73, 75, 75, 76, 73, 73, 76, |
| | AFA2- | | | 75, 73, 75, 74, 73, 76, 75, 74, 75, 72, 76, 79, 76, 77, 77, |
| | 4AA1- | | | 76, 77, 79, 79, 79, 80, 80, 83, 81, 77, 76, 75, 77, 78, 78, |
| | A4C3- | | | 78, 75, 90, 81, 81, 79, 74, 76, 76, 76, 79, 79, 80, 83, 82, |
| | E45DE12661DD | | | 80, 78, 80, 79, 80, 81, 78, 78, 80, 80, 78, 81, 80, 83, 78, |
| | | | | 80, 83, 79, 73, 85, 82, 77, 80, 74, 76, 76, 79, 88, 77, 80, |
| | | | | 77, 84, 78, 72, 57, 67, 75, 77, 77, 80, 74, 77, 77, 71, 76, |
| | | | | 75, 74, 78, 76, 77, 87, 76, 74, 72, 75, 73, 73, 74, 72, 73, |
| | | | | 73, 75, 76, 73, 73, 73, 75, 73, 73, 72, 75, 72, 71, 74, 73, |
| | | | | 75, 72, 70, 72, 71, 70, 72, 71, 73, 72, 73, 70, 70, 72, 70, |
| | | | | 70, 70, 68, 69, 91, 77, 71, 66, 67, 67, 66, 66, 64, 73, 69, |
| | | | | 67, 66, 68, 68, 79, 70, 69, 69, 65, 65, 67, 67, 71, 65, 78, |
| | | | | 70, 72, 70, 70, 65, 67, 68, 69, 73, 69, 73, 67, 66, 71, 66, |
| | | | | 71, 69, 70, 69, 70, 74, 71, 73, 70, 72, 71, 70, 68, 66, 71, |
| | | | | 71, 69, 67, 70, 70, 71, 70, 67, 68, 69, 69, 70, 69, 71, 72, |
| | | | | 68, 68, 70, 67, 72, 71, 71, 69, 73, 75, 95, 72, 73, 67, 64, |
| | | | | 68, 71, 68, 71, 70, 70, 69, 73, 71, 69, 72] |
| Epoxy | 649E3F49- | 8000 | 10500 | [114, 105, 100, 98, 101, 100, 100, 103, 102, 105, 103, |
| | 9C0C- | | | 104, 101, 101, 104, 107, 107, 101, 101, 100, 104, 103, |
| | 4AF5- | | | 102, 102, 104, 102, 102, 103, 102, 100, 102, 102, 102, |
| | A88C- | | | 104, 104, 100, 102, 103, 101, 100, 100, 99, 102, 102, |
| | D027B09F737D | | | 101, 102, 102, 101, 102, 105, 101, 102, 103, 102, |
| | | | | 103, 102, 102, 103, 103, 102, 102, 98, 98, 99, 100, 101, |
| | | | | 100, 100, 100, 102, 102, 100, 102, 100, 99, 102, 102, |
| | | | | 102, 100, 101, 103, 105, 101, 101, 101, 101, 104, 100, |
| | | | | 102, 104, 100, 108, 108, 99, 77, 97, 118, 105, 98, |
| | | | | 105, 95, 100, 99, 100, 105, 101, 101, 99, 100, 104, 101, |
| | | | | 98, 97, 101, 99, 99, 101, 101, 99, 99, 98, 101, 104, 99, |
| | | | | 99, 101, 95, 99, 101, 102, 102, 102, 99, 101, 102, 103, |
| | | | | 100, 99, 99, 102, 98, 102, 100, 101, 100, 99, 98, 100, |
| | | | | 102, 100, 102, 98, 96, 100, 102, 101, 100, 99, 103, |
| | | | | 101, 94, 99, 103, 99, 101, 98, 101, 99, 103, 100, 100, |
| | | | | 99, 99, 98, 102, 103, 140, 119, 115, 96, 102, 102, 100, |
| | | | | 100, 98, 101, 100, 99, 100, 103, 123, 123, 89, 117, 99, |
| | | | | 105, 103, 115, 100, 98, 98, 100, 100, 111, 105, 102, |
| | | | | 99, 98, 96, 98, 99, 100, 103, 103, 100, 102, 100, 101, |
| | | | | 101, 103, 100, 96, 101, 104, 101, 100, 98, 99, 95, 99, |
| | | | | 101, 105, 97, 99, 101, 100, 102, 101, 100, 100, 98, |
| | | | | 100, 98, 99, 99, 98, 100, 102, 98, 99, 99] |
| Polyurethane | EB3FE486- | 8000 | 10500 | [66, 72, 64, 64, 61, 60, 61, 58, 58, 59, 61, 62, 63, 65, 65, |
| | B926- | | | 64, 61, 60, 58, 57, 60, 59, 62, 62, 58, 60, 62, 63, 62, 62, |
| | 4E81- | | | 60, 58, 63, 62, 62, 60, 62, 60, 61, 61, 60, 58, 58, 56, 57, |
| | A1CD- | | | 57, 58, 58, 57, 58, 57, 60, 55, 54, 55, 55, 55, 54, 54, 55, |
| | 25A16183D5F0 | | | 54, 54, 54, 55, 53, 56, 55, 54, 55, 54, 55, 55, 55, 65, 104, |
| | | | | 57, 58, 54, 56, 55, 54, 55, 50, 55, 55, 58, 57, 57, 55, 56, |
| | | | | 54, 57, 56, 55, 55, 56, 53, 51, 55, 54, 53, 53, 53, 54, 53, |
| | | | | 53, 52, 51, 51, 50, 127, 55, 52, 51, 50, 52, 50, 48, 48, |
| | | | | 48, 47, 47, 48, 45, 45, 44, 45, 43, 43, 45, 42, 44, 43, 44, |
| | | | | 43, 44, 45, 43, 41, 42, 44, 42, 43, 42, 44, 41, 43, 44, 43, |
| | | | | 44, 42, 44, 44, 42, 112, 53, 51, 42, 42, 46, 45, 59, 59, 51, |
| | | | | 52, 41, 52, 73, 40, 59, 47, 45, 46, 52, 47, 45, 45, 46, 51, |
| | | | | 47, 49, 50, 53, 49, 48, 47, 50, 50, 52, 53, 53, 51, 51, 51, |
| | | | | 52, 55, 54, 57, 59, 53, 66, 57, 56, 56, 57, 57, 58, 58, 57, |
| | | | | 57, 59, 58, 62, 59, 68, 58, 64, 62, 59, 59, 63, 63, 62, 62, |
| | | | | 58, 61, 61, 62, 63, 59, 64, 63, 62, 62, 64, 148, 74, 71, |
| | | | | 72, 66, 62, 68, 66, 63, 60, 65, 66, 64, 62, 63, 77] |
| Polyurethane | BB08DDB8- | 8000 | 10500 | [61, 62, 61, 63, 64, 66, 66, 67, 64, 60, 57, 59, 59, 62, 61, |
| | DBC1- | | | 62, 59, 63, 60, 62, 64, 63, 61, 60, 90, 65, 62, 63, 61, 61, |
| | 4376- | | | 59, 62, 61, 60, 60, 60, 64, 61, 60, 61, 60, 61, 58, 57, 58, |
| | A117- | | | 58, 58, 56, 57, 57, 57, 57, 60, 55, 54, 54, 54, 56, 57, 53, |
| | B86C3AA83B22 | | | 55, 54, 53, 55, 52, 54, 55, 55, 55, 55, 53, 55, 56, 59, 152, |
| | | | | 57, 55, 54, 55, 56, 54, 56, 56, 50, 55, 54, 55, 58, 53, 55, |
| | | | | 56, 59, 54, 52, 51, 56, 53, 52, 53, 54, 57, 54, 53, 54, 52, |
| | | | | 53, 53, 53, 52, 51, 52, 51, 52, 51, 50, 50, 47, 50, 48, 48, |
| | | | | 48, 47, 47, 46, 46, 46, 48, 43, 46, 43, 42, 47, 43, 44, 44, |
| | | | | 44, 44, 43, 42, 43, 42, 45, 44, 43, 42, 42, 43, 42, 43, 44, |
| | | | | 44, 43, 46, 45, 92, 54, 52, 39, 43, 45, 45, 47, 46, 45, 45, |
| | | | | 44, 47, 44, 48, 45, 47, 48, 47, 47, 45, 45, 48, 48, 46, 50, |
| | | | | 46, 47, 52, 48, 50, 49, 49, 53, 52, 53, 51, 52, 52, 53, 54, |
| | | | | 55, 54, 53, 56, 58, 55, 57, 56, 54, 55, 59, 59, 58, 58, 59, |
| | | | | 58, 61, 61, 59, 69, 59, 65, 61, 59, 61, 61, 64, 62, 62, 57, |
| | | | | 63, 62, 63, 63, 61, 62, 64, 62, 63, 64, 111, 61, 79, 66, |
| | | | | 60, 64, 64, 69, 62, 66, 66, 61, 60, 62, 66, 78] |

Spectroscopy measurements ME as exemplified by 24 measurements in Table 1 above, may advantageously be used for training a classification model CM to classify new measurements into the surface characterizing classes CSS of binder-system alkyd, binder system epoxy and binder-system polyurethane, as well as a class of measurements unsuitable for classifying a surface SU, here referred to as “bad scans”. Further information selected from for example geolocation, e.g. established by a GPS receiver, date- and time-stamp for the measurement, configuration parameters, observations by the operator DO, local environmental circumstances during the measurement, e.g. temperature, humidity, surrounding light, etc., model number or serial number of the specific surface characterizing device system SCD performing the measurement, identification of the operator DO, etc. may in various embodiments be included in the spectroscopy measurement ME or the representation of the measurement MER for use by the recipient of the data, e.g. the training system TS for training classification models CM based on the spectroscopy measurements.

A corpus of measurements ME to use as training data may preferably encompass as much variation as possible within the desired surface characteristic classes SCC, in order to establish a robust and reliable trained classification model. Variation may for example be achieved by measuring different surfaces with the same coating system, different product variations (e.g. filler and pigment combinations) with the same binder system, variation in age of coating, variation in environmental exposure of coating, variation in mechanical damages of surface and coating, etc. In a particular embodiment, surfaces are measured as soon as freshly applied coating systems have cured and the measurement stored as a baseline ID profile of this coating system. Later measurements of the same surfaces provide ID profiles where similarities to the baseline ID profile may be indicative of the coating system, and differences to the baseline ID profile may be indicative of degradation and other changes caused by aging, damage, environmental exposure, etc.

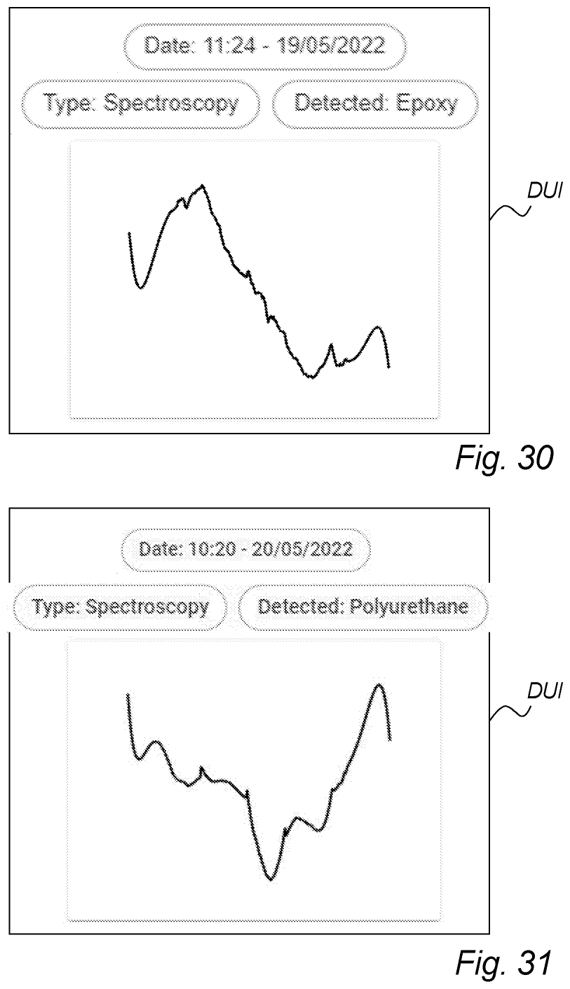

FIGS. 30-31 illustrate examples of user interfaces DUI of a surface characterising device SCD described above with reference to FIGS. 25-26. FIG. 30 illustrates a screenshot of a user interface DUI after performing measurement and classification of the spectroscopy measurement shown above in FIG. 28. The operator DO can see a measurement representation where the 250 measurement values have been smoothed, normalized and plotted as a graph, and the user interface also indicates the classification result, i.e. a determined surface characteristic class SCC being “Epoxy” in this case, and a date- and timestamp. Similarly, FIG. 31 illustrates a screenshot of the user interface DUI after performing a measurement and classification of the polyurethane-based coat with the spectroscopy measurement shown in FIG. 29.

The user interface DUI illustrated in FIGS. 30-31 may in various embodiments comprise more detail about the classification result, for example a percentage of classification certainty, or a list of alternative classes that came close. When the spectroscopy measurement ME is acquired with the purpose of training a classification model CM, the surface characterizing device system SCD and user interface DUI typically does not perform classification or show a classification result. Instead, the user interface may in various embodiments show more details about the spectroscopy measurement ME, such as any irregularities detected during measurement, e.g. not holding the device steady, not applying sufficient pressure, etc.

### Classification Model and Training

The present invention may involve a training system and a training method for training a classification model such that it may be capable of characterizing a surface comprising a cured coat, based on surface characteristic classes, for example using spectroscopy measurements. The invention may further involve a classification system and a method for characterizing the surface comprising a cured coat based on the surface characteristic classes, the trained classification model and for example spectroscopy measurements. The surface comprising a cured coat is characterized by means of the surface characteristic classes, which may e.g. include different binder system classes (sometimes referred to as class groups), such as binder classes, filler classes, pigment classes, coat manufacturer classes, coating product classes, contaminant classes, contamination state classes, degradation type classes, degradation state classes, and combinations thereof. Each class group comprises particular classes. E.g., the binder class may e.g. comprise classes representing different binder systems, such as, e.g., epoxy, polyurethane and alkyd etc. Similarly, other class groups may comprise different classes representing other surface characteristics.

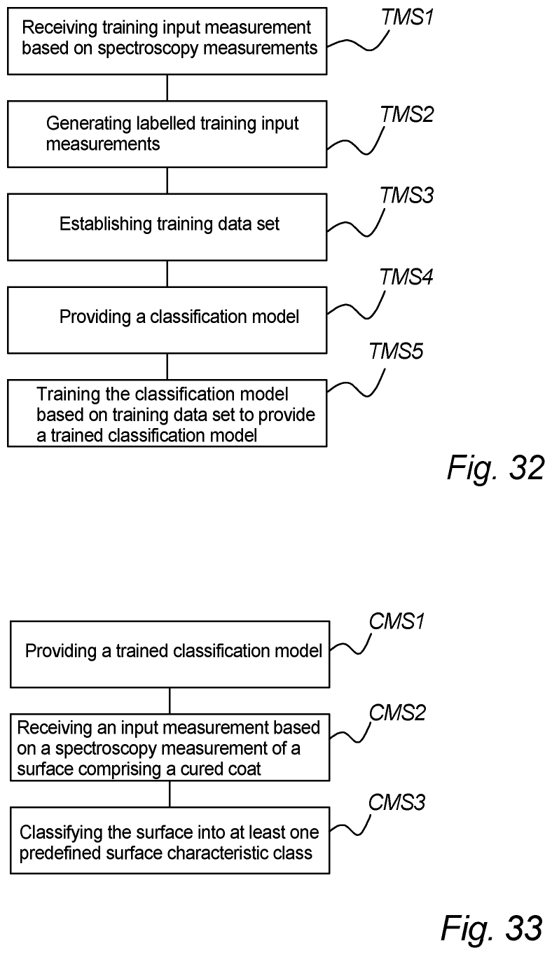

FIG. 32 illustrates training method steps TMS1-TMS5 of a training method for training a classification model according to an embodiment of the invention. The classification model is trained to enable the classification model to characterize a surface comprising a cured coat. The method is a computer implemented method in the sense that at least the particular step of training the classification model is computer implemented.

In a first training method step TMS1 training input measurements are received. The training input measurements are based on spectroscopy measurements ME of a training surface comprising a cured coat. Examples of spectroscopy measurements ME suitable as training input measurements are provided in Table 1 above.

In a further training method step TMS2, labelled training input measurements are generated by individually labelling the received training input measurements. The labelling is performed in accordance with a plurality of predefined surface characteristic classes. Hence, individual training input measurements are labelled with at least one of each surface characteristic classes, to produce labelled training input measurements. The one or more surface characteristic classes of each label are predefined at least in the sense that the surface characteristic class is not merely determined by the classification model but instead they are determined prior to the training of the classification model. The predefined surface characteristic classes associated with a particular training input measurement represents a surface characteristic of the surface comprising a cured coat, from which the training input measurement was acquired.

In an additional training method step TMS3, a training data set is established on the basis of the labelled training input measurements.

In a next training method step TM4, a classification model is provided. Examples of this will be given later herein.

In a final training step TMS4 of this exemplified embodiment of the invention, the provided classification model is trained on the basis of the training data set, to provide a trained classification model.

Advantageously, by training the classification model on the basis of the training data set with labelled training input data, relations between the training input data and the labels associated with each training input data may be modelled. Thereby, upon receiving an input measurement, e.g. a spectroscopy measurement ME, the trained classification model may be applied to classify the input measurement into at least one surface characteristic class.

Depending on the implementation of the invention, the training method steps described in relation to FIG. 32 may be performed in various different orders. For example, training method step TMS4 of providing the classification model may be performed as a first step of the training method or alternatively in between the step of generating labelled training input measurements TMS2 and the training method step TMS3 of establishing training data set.

The training method step TMS2 of generating labelled training input measurements may be performed manually by one or more individuals who labels the received training input measurements with a corresponding known surface characteristic class. Optionally, labelling the training input measurements may be performed by first classifying the training input measurements with a trained supervised classification model and/or with a trained unsupervised classification model and/or with a semi-supervised classification model, to obtain a temporary label based on one or more of the trained classification models, and then manually validating the obtained temporary label to obtain a validated label, which is then used to label the corresponding received training input measurement. In case the obtained temporary label is deemed incorrect in the manual validation, the person validating the temporary label may change the temporary label to the correct label that corresponds to the actual received training input measurement.

Optionally, training input measurements may be acquired from surfaces comprising a cured coat and wherein the surfaces further comprise a tag, such as, e.g., a visual label. The tag comprises information about the surface characteristic class of the cured coat of the surface. Advantageously, a tag reading system may read the tag and thereby label the training input measurements of the surface with the correct surface characteristic class based on the tag. The tag may, e.g., be a visual tag, e.g., comprising numerals, however it may also be a barcode, QR code or any other visual tag. An imaging system comprising a camera may then interpret the tag to obtain the surface characteristic class, which is used for labelling the training input measurements of the surface.

FIG. 33 illustrates classification method steps CMS1-CMS3 of a classification method of characterizing a surface, according to an embodiment of the invention. The surface comprises a cured coat, and the method is a computer implemented classification method.

In a first classification method step CMS1, a trained classification model is provided. The provided classification model has been trained based on a training data set comprising labelled training input measurements, which are essentially individual training input measurements that have been labelled with at least one of each surface characteristic classes. The training input measurements are based on spectroscopy measurements ME of training surfaces that is characterized by being associable with at least one predefined surface characteristic class of a plurality of surface characteristic classes. The training of the provided classification model may, e.g., be performed using the training method for training a classification model described in relation to FIG. 32.

In a next classification method step CMS2 an input measurement is received. The input measurement is based on a spectroscopy measurement ME of a surface SU comprising a cured coat. Input measurements may preferably be acquired using a surface characterizing device system SCD, for example as described with reference to FIGS. 7-31. Examples of spectroscopy measurements ME that may be used as input measurements for the classification method step CMS2 are provided in Table 1 above when disregarding the column “Binder-system class”.

In a further classification method step CMS3, the surface associated with the input measurement is classified into at least one of the predefined surface characteristics classes based on the input measurement, using the trained classification model. A classification output is produced from the model as a result of the classification, and the classification output is associated with at least one surface characteristic classes of the plurality of the surface characteristic classes.

Optionally, a surface may be classified as unknown. Classifying a surface as unknown may optionally be based on a certainty parameter associated with the classification model, and a certainty parameter threshold. As an example, a surface classified as unknown may, e.g., advantageously, be a surface that is different to the particular surface characteristics classes that the trained classification model has been trained to recognize and thereby classify. Advantageously, this has the effect of minimizing misclassifications of such unrecognizable surfaces.

Optionally, input measurements may be determined as unsuitable for classification (also referred to a bad scans or a bad acquisition), in which case, the classification method may not be performed. An input measurement may be determined as unsuitable for classification for various reasons. E.g., such bad scans may result from incorrect use of the surface characterizing device system during acquisition of the spectroscopy measurement. E.g., when the surface characterizing device system recognizes that an input measurement has been acquired with a pressure that does not comply with, e.g., a minimum pressure criterium or an acceptable pressure range. The pressure may e.g. be measured with a pressure sensor comprised by the surface characterizing device system. In an advantageous embodiment of the invention, incorrect use of the surface characterizing device system during measuring may also be determined based on one or more accelerometers configured to measure movement of the device during measuring, since too much movement may result in a bad scan. Also, any input measurements that deviate substantially from validated spectroscopy measurements may be characterized as bad scans. This could, e.g. include input measurements characterized by a spectrum with small peaks (amplitudes). Validated scans could e.g. be spectroscopy measurements that have been manually inspected and approved, including e.g. spectroscopy measurements utilized for training (training input measurements).

Optionally, the classification model may be retrained based on input measurements. In this case, the label associated with the input measurement is the class determined by the trained classification model. Advantageously, before applying input measurements and the associated class (label) for retraining the classification model, the validity of the associated class (label) may be evaluated. E.g. an input measurement may, e.g. be validated and used for retraining of the classification model if the certainty of the class, as provided by the classification model is sufficiently high. Alternatively, the validity may be evaluated based on standard known laboratory test procedures, to ensure that the classification provided by the classification model was correct. Other ways of ensuring the validity of the provided class of the input measurement may be utilized to validate the provided class associated with the input measurement, prior to using the input measurement and associated class as a training input measurement and label (a training example).

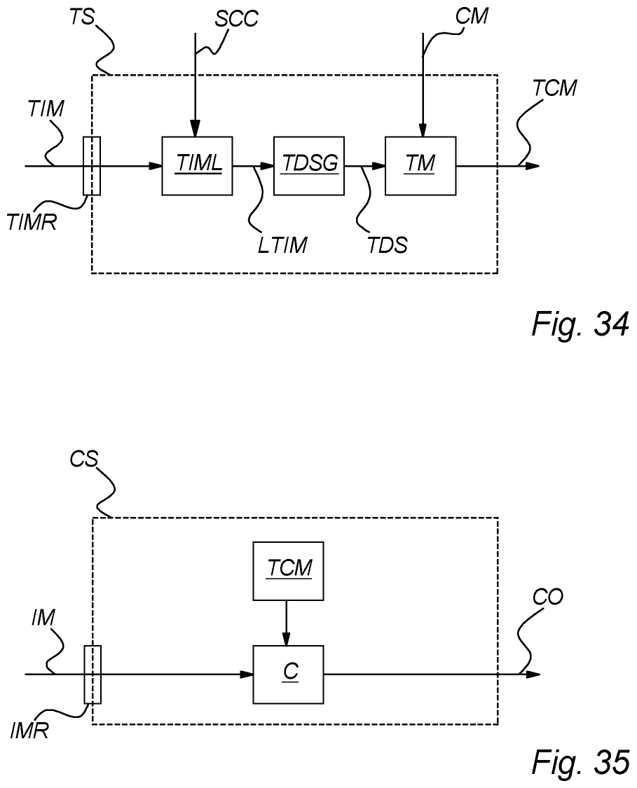

FIG. 34 illustrates a schematic representation of a training system TS according to an embodiment of the invention. Advantageously, the training system may be used to perform the training method described in relation to FIG. 32.

The training system TS is configured to train a classification model CM, such that the classification model CM becomes capable of characterizing a surface comprising a cured coat, by means of surface characteristic classes. The training is based on training input measurements TIM and surface characteristic classes SCC. The training system TS comprises a training input measurement receiver TIMR, a training input measurement labeller TIML, a training dataset generator TDSG and a training module.

The training input measurement receiver TIMR receives training input measurements TIMs, which are based on spectroscopy measurements ME of training surfaces comprising a cured coat. The training input measurements TIM are passed on to the training input measurement labeller TIML, which also receives a plurality of predefined surface characteristics classes SCC. The training input measurements TIM are then labelled with at least one of the predefined surface characteristic classes SCC, utilizing the training input measurement labeller TIML. The label thereby associates a spectroscopy measurement of a surface comprising a cured coat, with at least one predefined surface characteristic class, via the labelling of the training input measurement. See examples from one embodiment in Table 1, which may be considered representing labelled training input measurements LTIM, as it contains (all columns except the first column) spectroscopy measurements ME usable as training input measurements TIM, and contains (first column) a surface characteristic class SCC, in this specific example case a “binder-system class” which has been assigned to the training input measurement TIM to for the labelled training input measurement LTIM.

The training dataset generator TDSG collects the labelled training input measurements LTIM from the training input measurement labeller TIML, and generates a training data set on the basis of the labelled training input measurements LTIM. The training dataset TDS is then received by the training module TM along with the classification model CM, and the training module TM then trains the classification model CM based on the received training input dataset. The output from the training of the classification model is a trained classification model TCM.

Advantageously, the trained classification model TCM may be provided to a classification system, in which the trained classification model TCM may be utilized for classification of surface characteristics, based on input measurement.

FIG. 35 illustrates a schematic representation of a classification system CS according to an embodiment of the invention. Advantageously, the classification system CS may be used to perform the classification method described in relation to FIG. 33. The classification system CS is configured to characterize a surface comprising a cured coat. More specifically, the classification system is configured to classify a surface according to predefined surface characteristic classes, using an input measurement that is based on a spectroscopy measurement of the surface comprising a cured coat. The spectroscopy measurements may, e.g., be acquired using the surface characterizing device system, according to embodiments of the invention. The classification system CS comprises an input measurement receiver IMR, a classifier C, and a trained classification model TCM, which is trained at least based on a training data set, e.g., as previously described in relation to FIG. 32 and FIG. 34.

The input measurement receiver IMR receives an input measurement IM, which are based on a spectroscopy measurement (now shown) of a surface comprising a cured coat. The input measurement is passed on to the classifier C from the input measurement receiver. The classifier C then utilizes the trained classification model TCM to classify the surface into at least one of the predefined surface characteristics classes, based on the input measurement IM and using the trained classification model TCM. A classification output CO is produced from the classifier C as a result of the classification. The classification output CO is associated with at least one surface characteristic classes of the plurality of the surface characteristic classes.

In this exemplified embodiment of the invention, the classification output CO is the particular surface characteristic class classified by the classifier C based on the trained classification model TCM and the input measurement IM. Thereby, in this embodiment, the classification output CO is the surface characteristic class of the surface comprising a cured coat from which the input measurement IM was obtained.

In a first exemplified embodiment of the invention, the classification system CS is configured to classify binder type, including e.g. epoxy, alkyd and polyurethane based binder systems. Hence, as an example, the input measurement IM could be a spectroscopy measurement measured form a surface comprising a cured coating comprising an epoxy based binder. The input measurement is received by the classification system CS via the input measurement receiver IMR, which passes the input measurement to the classifier C, which provides the input measurement IM to the trained classification model TCM, which classifies the input measurement as an epoxy binder and outputs the class as a classification output CO. This particular trained classification model TCM has been trained based on a training data set comprising at least training input measurements of surfaces comprising an epoxy binder, and wherein these training measurements have been labelled with the class epoxy binder.

The training method and the training system illustrated in FIG. 32 and FIG. 34 may be implemented to train the classification model, and the classification method and classification system illustrated in FIG. 33 and FIG. 35, may be implemented to classify surface characteristic classes using the trained classification model. The classification model may be trained based on various different surface characteristic classes to provide a trained classification model that may be capable of classifying various different surface characteristic classes. As mentioned, the surface characteristic classes may comprise various different binder system classes, and hence, the classification model can be trained by the disclosed training system and/or by using the disclosed training method, to classify, e.g., various different binder system classes based on the disclosed classification method and/or classification system.

In a first example, labelled training input measurements are established by labelling the training input measurements according to binder-system class, which is selected at least among the binder-system classes epoxy, polyurethane and alkyd. A training dataset is then established based on the labelled training input measurements, and the classification model is trained using this particular training dataset, to provide a trained classification model. Since the model was trained based on the binder-system class comprising at least the classes epoxy, alkyd and polyurethane, the trained classification model is able to classify input measurements of surfaces comprising a cured coat into these particular classes.

In a second example, the labelled training input measurements are established by labelling the training input measurements according to coating manufacturer class, including, e.g., a coating manufacturer class such as ‘Hempel’. By performing the training method to train the classification model based on these particular labelled training input measurements, the trained classification model able to classify input measurements according to the manufacturer classes utilized in the training method.

In a third example, the training of the classification model is performed based on labelled training input measurements established by labelling the training input measurements according to degradation state classes. Various degradation state classes could be applied. Nonlimiting examples of degradation state classes may, e.g., comprise: no degradation, low degradation, medium degradation, high degradation, severe degradation. Thereby, advantageously, the trained classification model is able to classify input measurements according to the degradation state classes, utilized in the training method.

In a fourth example, the training of the classification model is performed based on labelled training input measurements established by labelling the training input measurements according to coating product classes. Examples of coating product classes may, e.g. comprise different coating products, e.g. referred to by different numbers and/or product names. The coating products may be of different manufacturers such as, e.g., Hempel. Thereby, advantageously, the trained classification model is able to classify input measurements according to the coating product classes, utilized in the training method.

In a fifth example, the training of the classification model is performed based on labelled training input measurements established by labelling the training input measurements according to degradation state classes. Various contamination classes could be applied. Nonlimiting examples of contamination classes may, e.g., comprise: salt content, water content, different particles generated during degeneration of the coating, etc. Thereby, advantageously, the trained classification model is able to classify input measurements according to the contamination classes, utilized in the training method.

In a sixth example, different surface characteristic classes may be combined to form new classes that is used to train the classification model. E.g. a surface characteristic class that combines binder type and filler type and/or pigment. An example of such a combined surface characteristic class could e.g. be a surface characteristic class that represents a coating comprising a certain binder, e.g., alkyd in combination with a certain filler and/or pigment. The classification model may thereby classify these combined classes.

In a seventh example, more than one classification model may be trained in parallel based on different surface characteristic classes, which advantageously enable the system to provide multi class classification in parallel based on two or more classification models. E.g. classify binder type and filler type, using two trained classification models. One trained to classify filler type and one trained to classify binder type. The models may be trained using the same training input measurements, however, the labels associated with the training input measurements would be according to the surface characteristic class that the model should be trained to classify.

Optionally, the classification model may comprise an unknown class, to enable the trained classification model to classify an input measurement into the unknown class. Advantageously, the unknown class enable classification into an unknown class when the input measurement represents e.g. a type of surface that is unknown to the trained classification model. Optionally, the unknown class may be utilized when the probability of the input measurement belonging to a certain binder system class is below a certainty threshold. Thereby, minimizing the false positive rate of the classification model.

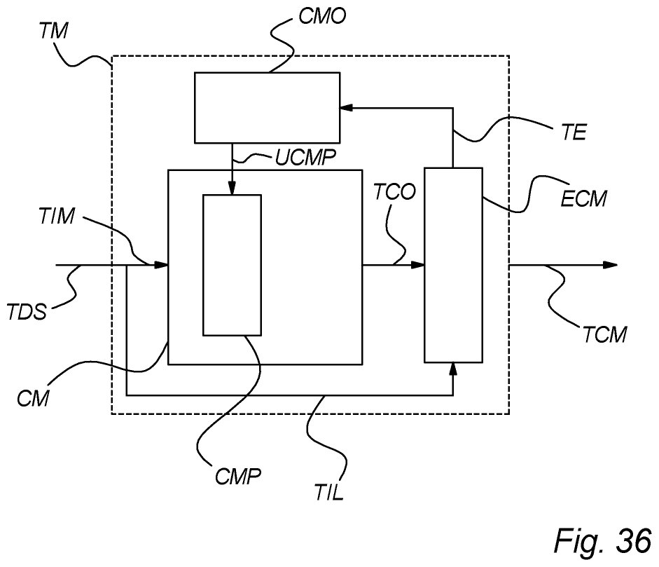

FIG. 36 illustrates a schematic representation of a training module TM according to an embodiment of the invention. The training module TM may, for example, be implemented as part of the embodied training system illustrated in FIG. 34. Advantageously, the training module TM may be used to train a classification model CM and hence, the training module TM may provide a trained classification model TCM based on the classification model, training input measurements and associated training input labels. Advantageously, the trained classification model TCM may, e.g., be used with embodiments of the classification system (not shown) of the invention and with embodiments of the classification method (not shown) of the invention, to classify surface characteristic classes based on input measurements. The operations performed by the training module TM may be computer implemented.

The training module TM comprises an error calculation module ECM, a classification model optimizer CMO, and a provided classification model CM comprising classification model parameters CMP.

In a first training iteration, the provided classification model CM receives training input measurements TIM from a training dataset TDS comprising labelled training input measurements. The classification model then classifies the training input measurements into one or more surface characteristic classes, and thereby provides a training classification output TCO comprising a classified surface characteristic class for each training input measurement. The error calculation module ECM receives the training classification outputs TCO and the training input labels TIL, each of which are associated with the corresponding training input measurements TIM from which the training classification output is generated. The error calculation module ECM compares the training classification output TCO with the training input labels TIL, to determine a training error TE. The training error TE represents a degree of classification wrongness, e.g. represented by a representation of a difference between the training classification outputs TCO and the associated training input labels TIL. The classification model optimizer CMO then adjusts the classification model parameters CMP to generate updated classification model parameters UCMP.

The classification model CM is initiated with an initial set of classification model parameters CMP, e.g. according to a model initialization strategy. The model initialization strategy may comprise various ways of selecting the initial classification model parameters, depending on the implementation of the invention. Nonlimiting examples of model initiation strategies may, e.g., comprise, sampling the initial set of classification model parameters CMP, e.g., from a statistical distribution such as e.g. from a Gaussian distribution, such as from a uniform distribution, such as from a truncated normal distribution. The classification model parameters CMP may, for example, be randomly selected (sampled) from the statistical distribution, or alternatively, be selected according to other selection strategies. E.g., the classification model parameters may in an alternative embodiment be initialized with equal parameter values across all the model parameters that is adjusted during training, e.g. with zeros, ones or other values. Alternatively and optionally, the model initialization strategy may comprise transfer learning, in which, the classification model parameters CMP are adopted from a pre-training of the classification model, wherein the pre-training of the classification model is based on different training data. Advantageously, this may improve the performance of the trained classification model.

Following the first training iteration; in a next training iteration, the classification model classifies the training input measurements TIM based on the updated classification model parameters UCMP, to produce a new training classification output TCO. The new training classification output TCO is received by the error calculation module ECM, which calculates a new training error based on the new training classification output and the training input labels TIL. The classification model optimizer CMO then provides updated classification model parameters UCMP. The training continues with further training iterations, wherein each training iteration provides updated classification model parameters.

Advantageously, the error calculation module ECM and the classification model optimizer CMO may cooperate to provide iterative training of the classification model CM, such that the classification model parameters are adjusted to minimize training error. This may be achieved in various ways according to the invention, such as, e.g., based on various types of optimization methods, e.g. including iterative optimization algorithms configured to update the classification model parameters such that the training error are minimized. The optimization algorithms may include calculating training error based on a cost function, wherein the cost function is dependent on the classification model parameters. Thus, determining a minimum of such cost function provides updated classification model parameters associated with a minimized training error (minimized cost) across the whole training dataset or across a batch of the training dataset. Nonlimiting examples of iterative optimization algorithms that may be implemented to provide updated classification model parameters UCMP may, e.g., include gradient descent types of algorithms, e.g. stochastic gradient descent, and other types of gradient descent types of algorithms. Alternative embodiments of the invention may also utilize different types of cost functions. The choice of cost function may depend on the implementation of the invention. Further nonlimiting examples of cost functions include least squares mean, multi-class cross entropy loss, and Kullback Leibler Divergence loss.

The optimization algorithm may comprise a learning rate parameter that specifies the step size for each iteration. More specifically, in each training iteration, the classification model parameters are updated stepwise, such that the cost function are minimized step by step towards a minima of the cost function. The learning rate specifies the size of each such step towards a minima of the cost function taken during an iteration of the optimization algorithm. Selecting a large learning rate may yield faster convergence of the model, meaning that determination of the updated classification model parameters UCMP that provides a minimal training error (minimal cost) is determined fast. However a large learning rate may result in overshoot of the minima. Selecting a smaller learning rate results in a slower descent towards the minima of the cost function, however, the chance of converging towards a global minimum of the cost function rather than towards a larger local minima of the cost function, is improved. The learning rate may be predetermined by a user, and/or the learning rate may be varied across training iterations. A user may experiment with different learning rates and select the learning rate that represents a good compromise between fast convergence, and an acceptable training error (cost).

Optionally, the optimization algorithm may comprise an adaptive learning rate. Non-limiting examples of optimization algorithms with an adaptive and/or varying learning rate include, e.g., root means squared propagation, which may be considered an extension of gradient descent and the AdaGrad version of gradient descent. Root mean squared propagation uses a decaying average of partial gradients in the adaptation of the step size for each parameter. Advantageously, the use of a decaying moving average allows the algorithm to focus on the most recently observed partial gradients seen during the progress of the search.

Optionally, a training termination condition may specify when to terminate training of the classification model. The training termination condition may be based on various conditions, including e.g. a predetermined number of training iterations, a predetermined training error, and a measure of the change in training error between one or more training iterations. The training termination condition may vary according to different implementations of the invention, and may further comprise combinations of one or more of, e.g., the mentioned training termination conditions and/or of other training termination conditions.

Optionally, the training data set TDS may be grouped into one or more training data batches that each comprises a subset of training data. The classification model may then be trained on each training data batch. This form of training may also sometimes be referred to as batch learning. The training may be performed iteratively, such that the classification model parameters from a previous iteration are utilized to initiate the next training iteration where training is performed with a next training data batch. Advantageously, this may improve the training error TE and thereby it may improve the performance of the trained classification model. The training may also be performed without initiating the model with the classification model parameters determined form a previous training based on a previous training data batch.

Optionally, the training of the classification model may be performed multiple times based on the same training dataset. Advantageously, this may improve the model performance. The number of passes of the entire training dataset that the classification model CM should completed during training of the model may be specified with the term ‘epoch’. Hence, one epoch indicates that the classification model CM has been trained based on one pass of the training dataset, two epochs indicates that the classification model has been trained based on two passes of the training dataset, etc. The optimal number of epochs may be determined in various ways depending on the implementation of the invention, including, e.g., based on the early stopping method. The number of epochs may, e.g., also be determined manually by continuing the training until the performance of the classification model does not improve further, as manually evaluated based on different model performance metrics. Model performance metrics may, e.g. advantageously be based on or more of the following metrics: confusion matrix, type I error, type II error, accuracy, recall, precision and F1-score, specificity, ROC (Receiver Operating Characteristics curve) curve, ROC curve AUC (area under the curve) score, PR score.

Optionally, the training termination condition may comprise a predetermined number of epochs.

Optionally the number of epoch may be based on a change in one or more model performance metrics. E.g., the training termination condition may specify a threshold value for difference in one or more model performance metrics between epochs. When that threshold (training termination condition) is exceeded and/or reached, the training is terminated.

Optionally, the model initiation strategy may include transfer learning, wherein the classification model parameters are initiated based on a pre-training using a pre-training training dataset. More specifically, the classification model parameters are adopted from a pre-training, wherein the classification model has been trained using pre-training training data. Advantageously, transfer learning enables the classification model to utilize knowledge gained from training on the pre-training training dataset to classify surface characteristics classes. A nonlimiting example of a pre-training dataset may, e.g., be the ImageNet dataset, which comprises a large amount of image-label pairs comprising an image of an object and an associated label that defines the class of the object of the image.

Optionally, the error calculation model and the training model parameter optimizer may be comprised by one module comprised by the training module. E.g., the classification model optimizer CMO may comprise the error calculation module, in some embodiments of the invention or the error calculation module may comprise the classification model optimizer.

Optionally, the training may be based on various types of backpropagation. This may, e.g., be advantageous when utilizing a neural network type classification model, including, e.g., convolutional neural network classification models, multilayer perceptron models, but also other types of neural network models.

It should be understood that in case a trained classification model performs worse based on testing compared to previous trained classification models, the model with the best performance may, advantageously, be utilized. Furthermore, training of a classification model may be based on a previous model that has already been trained. In fact transfer learning based on previous trained models may be applied, starting with any of the previously trained classification models, and not only the most recently trained model. Also, as the training data set grows, the classification model may be trained from scratch, without any prior knowledge from previous models. Multiple classification models may be trained and evaluated, and the trained classification model that provides the best performance may be selected and applied for classification of input measurements. E.g. by implementing this trained classification model in the surface characterising device, according to embodiments of the invention, and/or by implementing the classification model in a cloud computing environment, according to an embodiment of the invention. When implementing the trained classification model in a cloud computing environment, the classification model may be made accessible to various users via different computing devices, including e.g. the surface characterising device, smart devices, tablets, smartphones, laptops, personal computers etc.

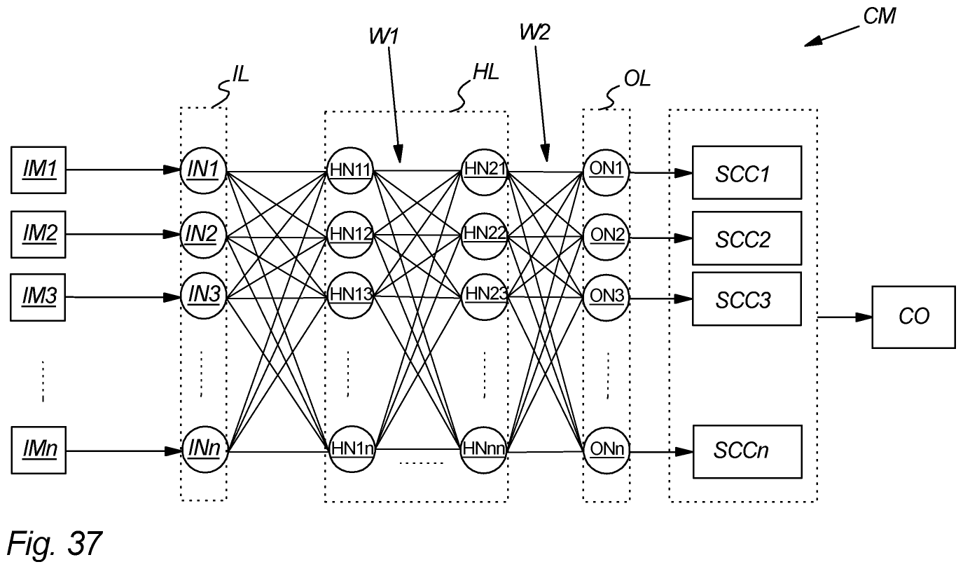

FIG. 37 illustrates a schematic representation of a classification model, according to an embodiment of the invention. More specifically, the illustrated classification model is an example of a neural network classification model. The neural network classification model may be trained using a training method such as the training method illustrated in FIG. 32, and using the training systems of the invention, including, for example, the training system illustrated in FIG. 34. When trained, the neural network classification model may be considered an example of a trained classification model according to the invention, which may be used in classification systems according to the invention, including, for example, the classification system illustrated in FIG. 35. Hence, when trained, the trained classification model may perform a classification method according to the invention, including the method described in relation to FIG. 33. The embodied neural network classification model consists of sets of neurons arranged in layers and wherein each neuron of a layer is connected with each neuron of the next layer, and with each neuron of the previous layer. In that sense, the neural network may be considered a fully connected neural network. Other embodiments of the classification model may include a neural network classification model that is not a fully connected neural network.

The neural network classification model illustrated in FIG. 37 comprises an input layer IL comprising a number of neurons IN1-INn, a number of hidden layers HL, and wherein each hidden layer comprises a number of hidden neurons HN11-HNnn. The network further comprises a number of output neurons ON1-ONn, and a set of weights W1, W2, wherein each connection between neurons in the hidden layer(s) and between a hidden layer and the output layer OL is associated with at least one weight. The individual weights of the sets of weights W1,W2 is illustrated as lines connecting the neurons. In addition to the weights, each layer except for the input layer may comprise a bias (not shown). The input layer IL is configured to receive input measurements IM1-IMn, for example measurements from Table 1 above, and further configured to pass these input measurements to the next layer, which is the first hidden layer HL. The hidden layers provides input to the neurons ON1-ONn of the output layer OL, which in turn outputs surface characteristics classes SCC1-SCCn. A classification output CO is then determined based on the surface characteristic classes SCC1-SCCn.

The neural network classification model may be trained based on a training dataset and based on a training algorithm such as, e.g. backpropagation including variations of training algorithms based on different types of backpropagation. Notice that other training algorithms may also be used, and that the skilled person would be able to select such different training algorithm if this would be beneficial. Nevertheless, alternatives to backpropagation based training algorithms may be less efficient. When the neural network classification model is trained, a trained classification model is obtained in form of a trained neural network classifier.

The trained neural network classifier may then be utilized to classify surface characteristic classes. In a classification scenario, the input layer of the trained neural network classification model receives input measurements IM1-IMn. The received input measurement are then passed to each neuron HN11-HN1n of a first hidden layer HL. Each neuron of the first hidden layer of the hidden layers HL then outputs a response based on the received input measurement, the weights W1 and a bias (now shown). The response is received by each neuron HN21-HNnn of the second hidden layer of the hidden layers HL, which each in turn outputs a response based on the received response from the first hidden layer, based on the individual weight associated with each neuron, and based on a second bias (now shown). The response of each neuron in the last hidden layer is received by each neuron in the output layer OL. Each neuron in the output layer then outputs a surface characteristic class SCC1-SCCn based on the response received from the last hidden layer and based on an output activation function.

In this example, the output activation function is a SoftMax function, which outputs a relative probability for each surface characteristic class SCC1-SCCn. In principle many other types of activation functions could be utilized as the output activation function.

In an optional step, a classification output CO may be determined based on the output of the output activation function. The classification output CO may e.g. be determined as the largest surface characteristic class value of all the outputted surface characteristic class values SCC1-SCCn. When a SoftMax activation function is utilized as output activation function, the largest surface characteristic class value would correspond to the surface characteristic class having the largest relative probability given the input measurements. This class would then be determined as the classification output.

The neural network classifier could be trained to classify different class groups including e.g. binder class, manufacturer class, degradation class and other class groups mentioned elsewhere in this disclosure. As an example, the neural network classification model could be trained based on training data associated with the binder class. Hence, the training data utilized to train such neural network classifier would comprise training input measurements that are based on spectroscopy measurements of a surface comprising a cured coat comprising a binder, and each of the training input measurements would be labeled with a specific binder from a binder class. The binder class could e.g. comprise epoxy, polyurethane, alkyd or other binder systems.

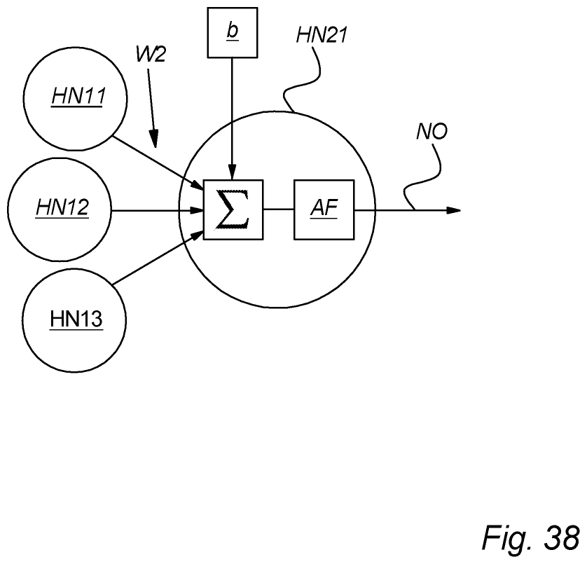

FIG. 38 illustrates a schematic representation of a neuron of a neural network classification model, according to an embodiment of the invention. The neuron could, e.g., be a neuron of a neural network classifier such as that illustrated in FIG. 37.

In principle the illustrated neuron could be an example of both a neuron of a hidden layer as well as an example of a neuron of an output layer of a neural network. The main difference between these two mentioned types of neurons being a difference in activation function AF. In this example the neuron is a neuron of a hidden layer. such as the hidden layer neuron HN21 of the neural network classifier illustrated in FIG. 37.

In this particular example, the hidden layer neuron HN21 calculates a weighted sum of the output from three hidden layer neurons of an upstream hidden layer HN11, HN12, HN13, using the weights W2. A bias is added to the weighted sum. Each layer comprises one bias parameter. Notice that in this example, W2 represents individual weights associated with each output from the hidden neurons HN11, HN12, HN13. The weighted sum with the bias added is then fed to an activation function AF, which calculates an output based on the received weighted sum plus the bias. The activation function can thereby be said to determine the output of the neuron. Some activation functions may e.g. be chosen to enable the neural network classifier to learn complex relationships such as, e.g., non-linear relations between an input measurement and the surface characteristic class associated with that input measurement. In an advantageous embodiment of the invention, each hidden layer neuron and each output neuron of the embodied neural network classification model comprises an activation function. However, in other embodiments of the invention not all neurons may comprise an activation function. Non-limiting examples of activations functions of hidden layer neurons of a neural network classification model that may be utilized to characterize surface characteristics includes, e.g., the sigmoid function, tanh function, exponential linear units, selfexponential linear units, the ReLU function (rectified linear unit), leaky ReLU function, parametric ReLU function, self-gated activation function, among others.

Advantageously, ReLU type functions have a derivative function and allows for backpropagation, while simultaneously making it computationally efficient. It further enables the neural network classification model to learn nonlinear relations. Further advantageously, since with ReLU functions only a certain number of neurons are activated, meaning that the output of the neuron is non-zero, the ReLU function is far more computationally efficient, e.g., when compared to e.g. the sigmoid and tanh functions. Furthermore, ReLU function accelerates the convergence of gradient descent towards the global minimum of the loss function due to its linear, non-saturating property.

In some situations, the ReLU function may results in dead neurons, e.g., neurons that outputs only zero values, thereby diminishing the flexibility and/or complexity of the neural network classifier. In this case, the leaky ReLu function, which has a small slope in the negative area of the function, may be applied instead to alleviate this problem. Alternatively, if the leaky ReLU function fails to alleviate the problem of dead neurons, the parametric ReLU function may be applied instead. The parametric ReLU function comprises a slope parameter that may be learned during backpropagation.

In deep neural networks, e.g., networks deeper than forty layers, the self-gated activation function may advantageously be applied.

The activation function of the output neurons of a neural network classification model according to an embodiment of the invention may comprise an activation function different to the activation functions applied in the hidden layers of the network. Examples of activation functions of the output layer comprises, e.g., sigmoid function, and the SoftMax function.

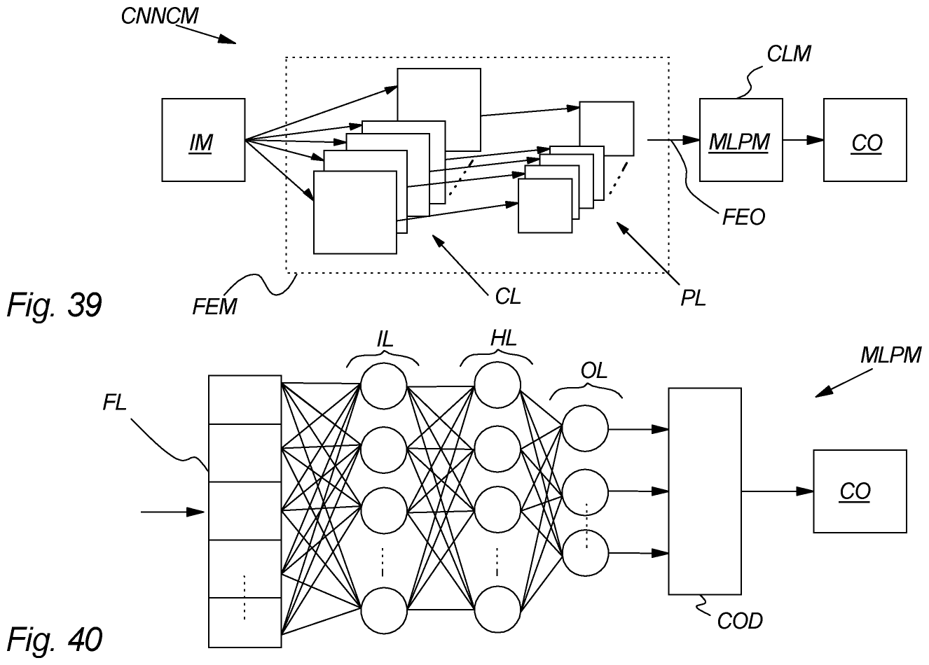

FIG. 39 illustrates a schematic representation of a classification model, according to an embodiment of the invention. More specifically, the illustrated classification model is an example of a convolutional neural network classification model CNNCM. The convolutional neural network classification model may be trained using a training method such as the training method illustrated in FIG. 32, and using the training systems of the invention, including, for example, the training system illustrated in FIG. 34. When trained, the convolutional neural network classification model CNNCM may be considered an example of a trained classification model according to the invention, which may be used in classification systems according to the invention, including, for example, the classification system illustrated in FIG. 35. Hence, when trained, the trained classification model may perform a classification method according to the invention, including the method described in relation to FIG. 33. In short, the convolutional neural network classification model CNNCM may advantageously be used to classify a surface characteristic of a surface comprising a cured coat, where the surface characteristic is classified as a surface characteristic class.

The convolutional neural network classification model CNNCM comprises a feature extraction module FEM followed by a neural network model NNM. The neural network model NNM classifies surface characteristic classes and generates a classification output based on a feature extraction output FEO received from the feature extraction module FEM. The feature extraction module FEM is configured to receive a matrix as input measurement IM, and to extract features of the matrix to generate the feature extraction output FEO. The matrix may, e.g., represent an image of plotted spectroscopy data, for example plots of the measurements in Table 1 above. The spectroscopy data may be obtained based on various different types of spectroscopy acquisition methods, as described elsewhere in this disclosure, including e.g. hyperspectral imaging. Hence, the matrix received by the feature extraction module may in an advantageous embodiment of the invention comprise spectroscopy data obtained based on hyperspectral imaging. The input measurements may be raw spectroscopy data, however, the input data may also be pre-processed spectroscopy data which have been pre-processed in different ways, e.g., smoothed, filtered, converted, normalized, etc. The required matrix format of the input measurements may be achieved by generating a matrix representation of the spectroscopy measurements if the raw or preprocessed spectroscopy data is not already arranged in a matrix format, for example, by generating a binary image of plotted spectroscopy measurements, such as an image of a graph representing the spectroscopy measurements. Alternatively, the spectroscopy measurements may be represented by a color image. In the latter example, the input measurement would be a three dimensional matrix representing the area of the image in two dimensions, while the third dimension would typically comprise the three color channels: red, green and blue. In any case, the image of the plotted spectroscopy measurements becomes at least a two dimensional representation of the actual spectroscopy measurements. Spectroscopy data in a string format, may also be reshaped to matrix format to be used as input measurements to the convolutional neural network classification model CNNCM. The input measurements in a matrix format may sometimes comprise more dimensions than the red, green, blue color image just described. For example, when the spectroscopy measurements are obtained based on hyperspectral imaging, many wavelengths may be acquired, in which case, the matrix representation of the input data may comprise multiple dimensions. When using hyperspectral imaging data as input measurement, the matrix representation may e.g. comprise two dimension representing the actual area of the image, and multiple further dimensions each representing, e.g., a portion and/or a wavelength of the electromagnetic spectrum. Independent of which kind of spectroscopy measurements that is utilized with the convolutional neural network classification model CNNCM, the matrix representation of the spectroscopy measurements is received by the feature extraction model as input measurements IM.

The feature extraction module FEM comprises at least one convolutional layer CL, and optionally a following pooling layer. The convolutional layer comprises at least one kernel (sometimes also referred to as a filter). Typically, the feature extraction module may perform better when using multiple kernels in each layer, since this enables the feature extraction model to learn more features of the input measurements. For similar reasons, the classification model also typically comprises more than one convolutional layer, since this enables the model to learn more complex features of the input measurements, and hence, enable the model to perform better. Each kernel of a layer is individually convolved with the input measurement IM, to extract features from the input measurement IM. If the model comprises more than one convolutional layer, the output of the first convolutional layer is received by the next convolutional layer, and so forth. Again optionally, the output of a convolutional layer is typically received by a pooling layer, as mentioned above. So, typically, a convolutional layer comprises a plurality of kernels to enable extraction of a plurality of features, to enable better performance of convolutional neural network classification model CNNCM. The kernel is a small matrix with a size that is less than the size of the input measurement. The size of the kernel may vary depending on the particular implementation of the embodiment of the invention, and may, e.g., be based on empirical testing of the model with different filter sizes. The filter is moved across the height and width of the input measurements and the dot product of the kernel and the image are computed at every spatial position of the filter. The length by which the kernel slides across the input measurement IM is the stride length. Different stride lengths may be tested to determine the stride length that provide the optimal performance of the convolutional neural network classification model CNNCM. The stride length may be part of what is sometimes referred to as the hyper parameters of the model. The hyperparameters may be tuned based on evaluation of the model performance, e.g. based on empirical testing of the model, wherein the hyper parameters are varied for each training and test iteration, and ultimately, the best performing model of the trained and tested models are chosen as the trained classification model. The actual coefficients of the kernels are determined based on training of the network, and the training of the network is performed following the previously described methodology, and thereby the training is based on a training dataset with labeled training input measurements. Optionally, when the convolutional layer comprises multiple kernels, the output of each kernel may be stacked, and an activation function may be applied to the stack of kernel outputs. Advantageously, by applying activation functions, the classification model may learn complex non-linear features of the input measurements, as previously discussed in relation to the neural network classification model described in relation to FIG. 37. Various different types of activation functions including, e.g., ReLU functions and/or tanh functions may be used, however, other activation functions may also be utilized, as discussed elsewhere in this disclosure.

Following a convolutional layer, the pooling layer PL may optionally be arranged to receive the output of the convolutional layer CL. The pooling layer reduces the size of the feature extraction output (sometimes referred to as feature maps) outputted by the convolutional layers and thereby may speed up the computation of training and surface characteristic classification of the classification model. Advantageously, max pooling may provide a good performance in the context of characterizing a surface comprising a cured coat. Nevertheless, embodiments of the invention is not limited to using max pooling, and so, e.g., average pooling and other types of pooling may also be utilized, depending on the particular implementation of the invention. In short, using max pooling; from each patch of a feature map (the output of a convolution with a kernel of the convolutional layer CL), the maximum value is selected to create a feature map with a reduced size. In average pooling; from each patch of a feature map (the output of a kernel of a convolutional layer CL), the average value is selected to create a reduced feature map. The size of the map outputted by the pooling layer depend on the size of the patch applied in the pooling layer, and may further depend on the stride length of the applied patch. To reduce computer resources required to perform a surface characteristic classification using the convolutional neural network classification model and the computer resources required to train the classification model, the patch size and stride length may be determined such that the output of the pooling layer reduces the size of the feature map received from the convolutional layers substantially. On the other hand, reducing the feature maps may result in loss of information and degraded performance of the classification model. The optimal hyper parameters, e.g. the stride length and patch size (sometimes referred to as kernel) of the one or more pooling layers may be selected by training the model using different hyper parameters and then comparing each of the trained model, and then selecting the classification model with the best classification performance, or alternatively, selecting the model with the best compromise between classification performance and required computer resources.

The feature extraction output of the feature extraction module is the result of the input measurement being convolved with kernels of the convolutional layer and with the kernels of the pooling layers of the feature extraction module. The feature extraction output is flattened to a vector. Then this vector is fed to a classification module CLM, which in this exemplified embodiment comprises a neural network model, e.g. a multilayer perceptron model MLP, the output of which is the classification output. The classification module may in other embodiments of the invention comprise other types of classifiers.

FIG. 40 illustrates a schematic representation of a neural network model. In this exemplified embodiment of the invention, the neural network model is a fully connected neural network model (sometimes referred to as a multilayer perceptron model MLPM). Nevertheless, other embodiments of the invention may utilize neural network models that is not fully connected neural network models. The multilayer perceptron model MLPM of this embodiment may, for example, be utilized in the classification module CLM of the convolutional neural network classification model CNNCM illustrated in FIG. 39, for producing a classification output CO comprising one or more surface characteristic classes, based on a feature extraction output received from a feature extraction module FEM.

In this exemplified embodiment, the multilayer perceptron model MLPM comprises a flattening layer FL, which flattens the feature extraction output into a vector, an input layer IL, a hidden layer HL, and an output layer OL, which are all fully connected. After the multilayer perceptron model MLPM, a classification output determiner COD is arranged to receive the output of the output layer OL, and based on this output, determine a classification output CO. The classification determiner COD is an optional feature, which may be omitted in embodiments of the invention, when it is preferred to obtain the output of the output layer. E.g. when it is preferred to obtain a relative probability of an input measurement belong to each of the surface characteristic classes. This may e.g. be achieved when the output layer comprises a SoftMax activation function.

The flattening layer flattens the received feature extraction output FEO to a vector and then feeds each value of the vector to each neuron in the input layer of the multilayer perceptron model MLPM. Weights, bias, and activation functions of the layers are then applied as previously described in relation to the description of the neural network model illustrated in FIG. 37, and in relation to the description of a neuron of a neural network illustrated in FIG. 38.

The output of the output layer is the relative probability for each surface characteristic class, including for example, binder types such as epoxy, polyurethane, alkyd and so forth. The output of the output layer is received by the classification output determiner COD. The classification output determiner COD then selects a classification output comprising a surface characteristic class. In this example, the classification output is selected by choosing the surface characteristic class with the highest probability. The classification output determiner COD may alternatively be configured to bypass the output of the output layer.

Notice that the training of the convolutional multilayer perceptron model MLPM is performed for the full model, including both the multilayer perceptron model multilayer perceptron model MLPM and the feature extraction module comprising the convolutional layer(s) and the pooling layer(s). Thus, when training the convolutional neural network classification model CNNCM, the training data comprises training examples which consists of training input measurement and an associated known label comprising the actual surface characteristic class of that training input measurement.

In an advantageous embodiment of the multilayer perceptron model MLPM, the model comprises more than one hidden layer. This advantageously has the effect that the model is capable of learning more complex relations between training input measurements and associated labels, and thus it may elevate the performance of the multilayer perceptron model MLPM.

In this exemplified embodiment, the multilayer perceptron model MLPM is illustrated with four neurons in the hidden layer HL. However, as illustrated by the dots shown between the bottom two neurons of the hidden layer HL, the multilayer perceptron model MLPM may comprise additional neurons. Including more neurons may advantageously increase model performance. Increasing the number of neurons in the network may, however, diminish computational speed. Empirical testing of different model architectures enables the user to select a model with a good ratio between performance and computational speed.

Notice that sometimes a neuron may also be referred to as a perceptron.

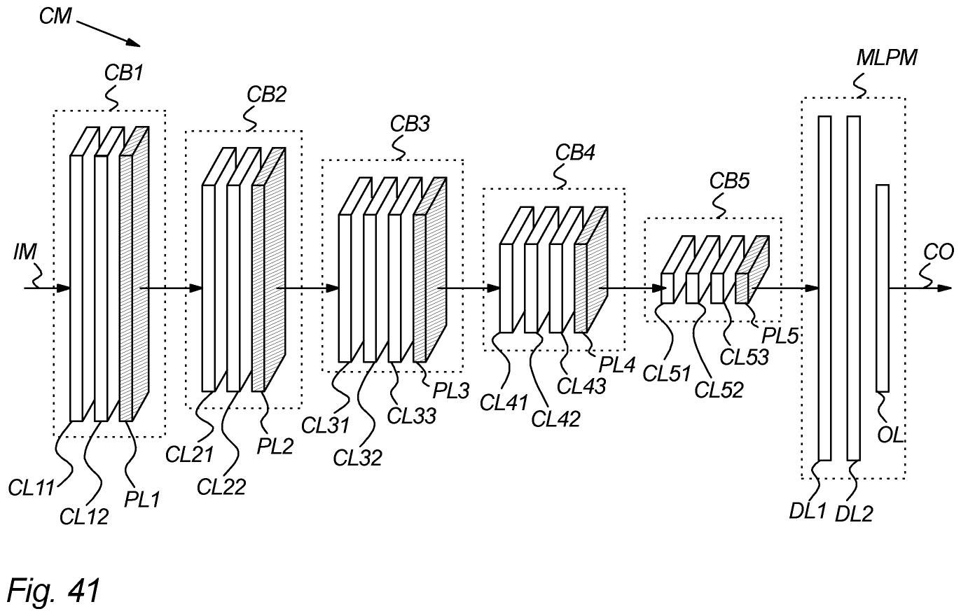

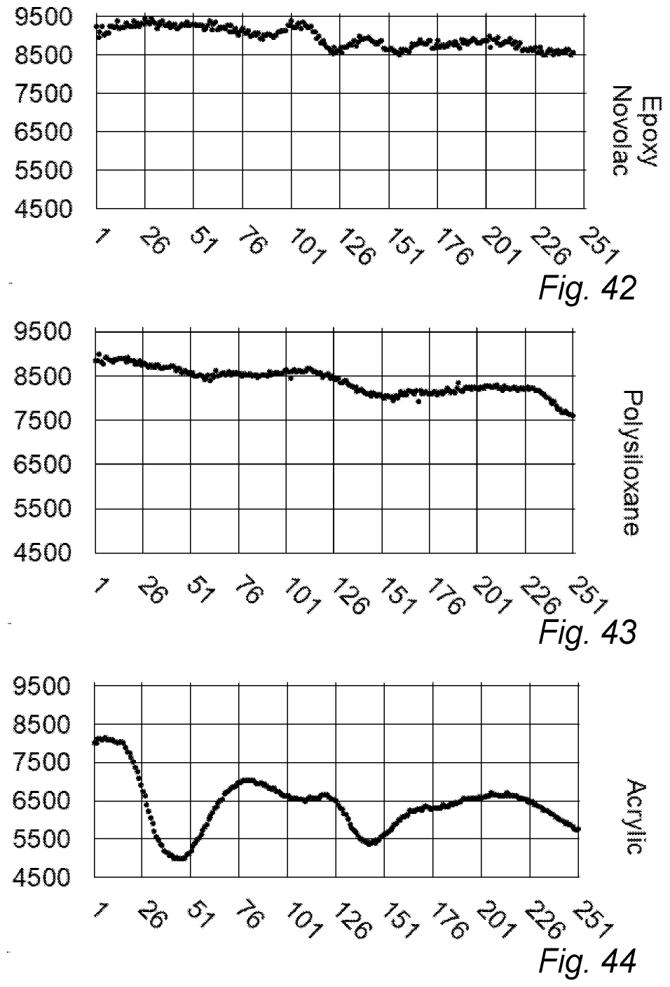

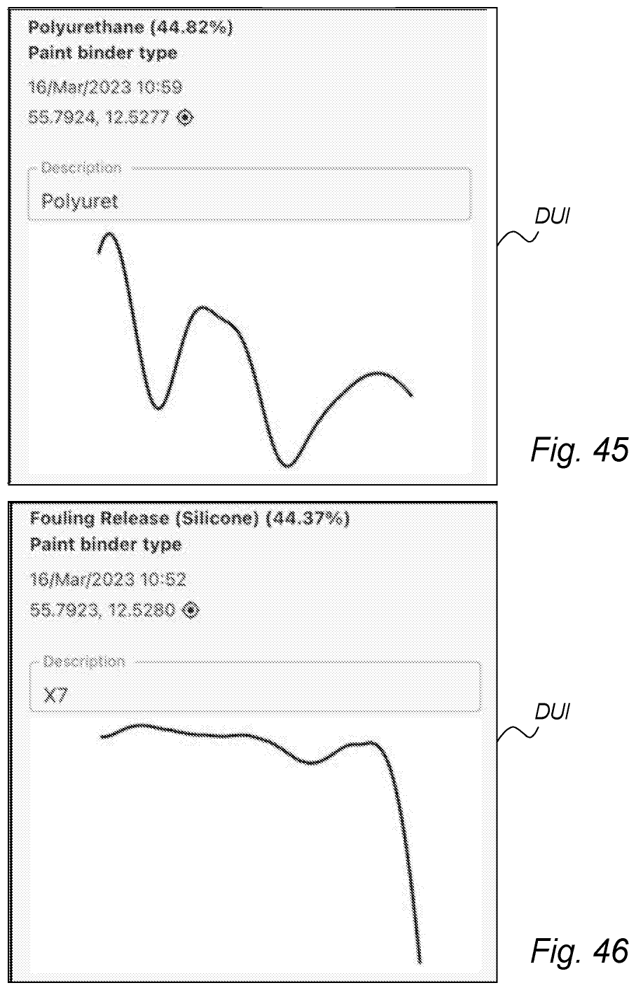

FIG. 41 illustrates an example of a classification model CM implemented as a convolutional neural network classification model, according to an embodiment of the invention. The particular convolutional neural network classification model illustrated in FIG. 41 may sometimes be referred to as VGG16. VGG16 can be considered an example of the convolutional neural network classification model described in relation to FIG. 39. As such, VGG16 requires training input measurements and input measurements to be received in a matrix format as previously described in relation to the generic convolutional neural network classification model illustrated in FIG. 39. Thus, the training input measurements and input measurements applied for use with VGG16 may, e.g., be a red, green, blue image of an image taken of plotted spectroscopy measurements, for example as illustrated in FIGS. 30-31.

The VGG16 convolutional neural network classification model may be trained using a training method such as the training method illustrated in FIG. 32, and using the training systems of the invention, including, for example, the training system illustrated in FIG. 34. When trained, the VGG16 convolutional neural network classification model may be considered an example of a trained classification model according to the invention, which may be used in classification systems according to the invention, including, for example, the classification system illustrated in FIG. 35. Hence, when trained, the trained classification model may perform a classification method according to the invention, including the method described in relation to FIG. 33. In short, the VGG16 convolutional neural network classification model may advantageously be used to classify a surface characteristic of a surface comprising a cured coat, where the surface characteristic is classified as a surface characteristic class.

The VGG16 classification model comprises 16 layers with weights that may be determined through training of the classification model. These layers include 13 convolutional layers followed sequentially by a multilayer perceptron model comprising three dense layers DL including the output layer OL. The padding of the convolutional layers is such that the spatial resolution is preserved after convolution (also referred to as same padding). The convolution stride length is set to one pixel. In addition to the convolutional layers, VGG16 comprises five max-pooling layers, which follows some of the convolutional layers. Max-pooling is performed over a 2-by-2 pixel window, with a stride of two. The ReLU activation function is used for each of the convolutional layers and each of the dense layers

In a sequential order from the first convolutional layer to the last output layer of the multilayer perceptron model, the VGG16 model comprises five convolution blocks CB1-CB5.

The first convolution block CB1 comprises two convolution layers CL11, CL12 of 64 channels (channel refers to the number of filters in a layer) with a kernel size of 3-by-3 and same padding, and a max-pooling layer PL1 of 2-by-2 pool size and a stride of two. The first layer CL11 of the first convolution block CB1 receives input measurements. The input measurements are required to be in a matrix format.

The first convolution block CB1 is connected to the second convolution block CB2, which comprises two convolutional layers CL21, CL22, each having 128 channels of 3-by-3 kernels and same padding, and a max-pooling layer PL2 of 2-by-2 and a stride of two.

The second convolution block CB2 is connected to the third convolution block CB3, which comprises three convolutional layers CL31, CL32, CL33, each having 256 channels of 3-by-3 kernels and same padding, and a max-pooling layer PL3 of 2-by-2 and a stride of two.

The third convolution block CB3 is connected to the fourth convolution block CB4, which comprises three convolutional layers CL41, CL42, CL43, each having 512 channels of 3-by-3 kernels and same padding, and a max-pooling layer PL4 of 2-by-2 and a stride of two.

The fourth convolution block CB4 is connected to the fifth convolution block CB5, which comprises three convolutional layers CL51, CL52, CL53, each having 512 channels of 3-by-3 kernels and same padding, and a max-pooling layer PL5 of 2-by-2 and a stride of two.

The fifth convolution block CB5 is connected to the first dense layer of the multilayer perceptron model MLPM via a flattening layer (not shown), which is configured to flatten the output of the fifth convolutional block CB5, meaning that the output of the fifth convolutional block is transformed into a vector. The vector is then received by a first dense layer DL of the multilayer perceptron model MLPM, which comprises 256 neurons. The first dense layer DL1 is connected to the second dense layer DL2, which comprises 128 neurons that is fully connected to the first dense layer DL1. The output of the second dense layer is connected to the output layer OL, which comprises a number of neurons corresponding to the number of surface characteristic classes that the model should be able to predict. The neurons of the output layer are fully connected to the neurons of the second dense layer DL2. The output layer outputs a classification output CO, which is the relative probability for each of the surface characteristic classes given the input measurement IM. Each of the neurons of the output layer OL represents a surface characteristic class. The relative probability of the surface characteristic classes is calculated by the soft-max activation function, utilized by the output layer OL.

Being a convolutional neural network type classification model, the VGG16 classification model requires the input measurements to be in matrix format.

Optionally, classification models of the invention may be trained based on categorical crossentropy loss function. Further optionally, the RMSprob may be utilized to control learning rate. Also, optionally, class weights may be applied to advantageously handle imbalance.

Optionally, transfer learning may be applied to the VGG16 classification model. Transfer learning may be based on, e.g., the ImageNet dataset.

In addition to the classification models described in relation to the figures, other supervised classification models may advantageously be utilized in different embodiments of the invention. The classification models may receive input measurements in the form of raw input measurements or in the form of input measurements that have been preprocessed in various ways. Furthermore, the classification models may not necessarily be a type of neural network. Indeed, other types of classification models may provide solid performance, e.g., when training data is limited. Non-limiting examples of such other types of classification models that may advantageously be used in embodiments of the invention includes, e.g., various types of decision trees, support vector machine models, Bayesian statistical models, logistic models, gradient boosting models including, e.g., XGBoost. Notice that these models may be trained using the training system of the invention. Furthermore, e.g., elastic net may optionally be used to handle regularization and to reduce the weight of “useless” features. Also, optionally principle component analysis PCA may be utilized to group features based on variance and thereby reduce the size of the input to the classification model. Thereby, advantageously reducing the computational requirements of the model and improving the computational speed of the classification of surface characteristic classes.

### Classification Model Testing

The performance of the classification models of the various embodiments of the invention may be evaluated based on different model performance metrics. Model performance metrics may, e.g., advantageously be based on or more of the following metrics: confusion matrix, type I error, type II error, accuracy, recall, precision and F1-score, specificity, ROC (Receiver Operating Characteristics curve) curve, AUC (area under the curve) score, PR score, etc. The model performance may advantageously in some embodiments of the invention be automatically evaluated by a test module, which may calculate one or more of the model performance metrics.

Hyper parameters of the classification models may be tuned (sometimes also referred to as adjusted) in different ways. Optionally, the hyper parameters of the classification models may, e.g., be adjusted based on Grid and/or random search for optimal parameters, and/or based on cross validation. Other types of methods for adjusting hyper parameters may also be applied, according to different embodiments of the invention.

Optionally neural network based classification models may advantageously utilize one or more normalization layers, such as e.g., dropout layer. Advantageously this has the effect of regularizing the classification model, and thereby, it may improve the performance of the classification model, e.g. by avoiding overfitting to e.g. the training data.

### Example 1

In a first example, the VGG16 classification model was trained and tested in regards to classifying surface characteristic classes based on spectroscopy measurements acquired from surfaces (training surfaces) coated with actual coating products comprising different binders, fillers, pigments etc, using the surface characterizing device system (see the description referencing FIGS. 25 and 26) implemented with single bounce ATR spectroscopy. In this particular example, the specific surface characteristic classes included the binders alkyd, class 2, epoxy and polyurethane.

Using the surface characterizing device system, spectroscopy measurements of 200 training surfaces comprising a cured coat with a known binder were acquired with single bounce ATR spectroscopy. For each of these spectroscopy measurements, 250 samples were acquired over the wavelength spectrum of 8000 nm-10500 nm. Based on a manual evaluation of the measurements, 19 measurements were discarded as unsuitable (bad scans) for training and testing of the classification model. The remaining 181 spectroscopy measurements were included for training and testing of the classification model.

In a next step, the 181 individual spectroscopy measurements were pre-processed. Pre-processing included smoothing the spectroscopy measurements using a Savitzky-Golay filter with a window length of 41 and a polynomial order of 5. Then, the smoothed spectroscopy measurements were plotted and a red, green, blue colour image (RGB image) of the plotted spectroscopy measurements were acquired. Two examples of the smoothed and plotted spectroscopy measurements are shown in FIGS. 30-31. In a next pre-processing step, the RGB image of the plotted and smoothed 250 samples spectroscopy measurements were transformed into a 300-by-300-by-3 dimensions array and all values of the array were rescaled to a range between zero and one.

The pre-processed spectroscopy measurements were then split into a test dataset comprising 73 pre-processed spectroscopy measurements and into a training data set comprising 108 pre-processed spectroscopy measurements, using random split. Since the spectroscopy measurements were acquired from surfaces coated with a coating comprising a known binder, each spectroscopy measurement was labelled according to the binder as either epoxy, alkyd, class 2 or polyurethane, to produce labelled spectroscopy measurements, wherein the portion of these utilized for the training data set was denominated as labelled training input measurements.

The VGG16 model was adopted as classification model. In this example, transfer learning was applied in the sense that the VGG16 model was pretrained based on the publicly available ImageNet dataset prior to being trained on the training data set comprising the 108 examples of labelled training input measurements. The pretrained VGG16 was then trained based on the training data set, using backpropagation and the categorical cross entropy loss function. Root mean square propagation (RMSprob) was used as optimizer and class weights were used to handle imbalance. Further training specifications include a batch size of 15 and an epoch of 30.

The performance of the trained VGG16 classification model was then evaluated based on the test data set, wherein the VGG16 model was applied to classify the labelled test measurements of the test data set, and wherein the classification was compared with the correct label associated with each labelled test measurement. The performance of the VGG16 classification model was evaluated based on a confusion matrix (see table 2 below), and further based on the performance metrics: recall, precision and F1 (see table 3 below).

Table 2 below shows the confusion matrix generated based on the test data set. The confusion matrix shows the classification of the test measurements in relation to the actual true label of the test measurement. Based on table 2, it may e.g., be appreciated that the trained VGG16 classification model correctly classified 32 of the 38 test measurements comprising an Epoxy binder correctly as Epoxy, while only 4 test measurements were wrongly classified as alkyd and 2 as polyurethane.

TABLE 2

| | True surface characteristic class | | | | |
|---|---|---|---|---|---|
| | | Alkyd | Class 2 | Epoxy | Polyurethane |
| Classified | Alkyd | 9 | 0 | 4 | 2 |
| surface | Class 2 | 0 | 4 | 0 | 1 |
| characteristic | Epoxy | 3 | 1 | 32 | 2 |
| classes | Polyurethane | 0 | 0 | 2 | 13 |

Table 3 below shows the performance of the VGG16 classification model as measured based on the test data set. The applied performance metrics (recall, precision and F1) are well accepted performance metrics widely applied to measure performance of classification models. From Table 3, it may e.g. be appreciated that the trained VGG16 classification model had a recall of 84%, a precision of 84% and an F1 score of 84% for the epoxy binder class. Further notice, that the Macro avg parameter of Table 3 is the average score for the metrics across all four classes. E.g., the average precision of the trained VGG16 classification model was 78% across all four classes. The three performance metrics were calculated as follows:

$$Recall = TruePositives / ( TruePositives + FalseNegatives )$$

$$Precision = TruePositives / ( TruePositives + FalseNegatives )$$

$$F 1 = ( 2 ⋆ Precision ⋆ Recall ) / ( Precision + Recall )$$

TABLE 3

| Label | Recall | Precision | F1 | Images |
|---|---|---|---|---|
| Alkyd | 75% | 60% | 67% | 15 |
| Class 2 | 80% | 80% | 80% | 5 |
| Epoxy | 84% | 84% | 84% | 38 |
| Polyurethane | 72% | 87% | 79% | 15 |
| Macro avg | 78% | 78% | 77% | 73 |

### Example 2

In a second example, a neural network as described above with reference to FIGS. 37-38 is trained and tested for classifying surface characteristic classes based on spectroscopy measurements acquired from surfaces (training surfaces) coated with actual coating products comprising different binders, fillers, pigments etc, using the surface characterizing device system as described above with reference to FIGS. 25-26. Spectroscopy measurements of training surfaces comprising a cured coat with a known binder are acquired with single bounce ATR spectroscopy. For each of these spectroscopy measurements, 250 samples are acquired over the wavelength spectrum of 5,500 nm-8,000 nm. A part of the measurements are after manual evaluation discarded as unsuitable (bad scans) for training and testing of the classification model.

The remaining spectroscopy measurements are pre-processed and split into a test dataset and a training data set using random split configured to assign a major part of the pre-processed spectroscopy measurements to the training data set. Since the spectroscopy measurements are acquired from surfaces coated with a coating comprising a known binder, each spectroscopy measurement are labelled according to the binder, to produce labelled spectroscopy measurements, wherein the portion of these utilized for the training data set are denominated as labelled training input measurements.

The neural network is then trained based on the training data set, using backpropagation, and the performance of the trained classification model is evaluated based on the test data set, wherein the neural network is applied to classify the labelled test measurements of the test data set, and wherein the classification is compared with the correct label associated with each labelled test measurement. The performance of the classification model is evaluated based on a confusion matrix, and further based on the performance metrics: recall, precision and F1.

A confusion matrix is generated based on the test data set. The confusion matrix shows the classification of the test measurements in relation to the actual true label of the test measurement. The applied performance metrics (recall, precision and F1) are well accepted performance metrics widely applied to measure performance of classification models. The three performance metrics are calculated as follows: Recall=TruePositives/(TruePositives+FalseNegatives); Precision=TruePositives/(TruePositives+FalsePositives); and F1=(2*Precision*Recall)/(Precision+Recall).

### Accelerated Coat Degradation

The training data described and provided by examples above are acquired by performing spectroscopy measurements on relatively freshly coated surfaces with a very low degree of degradation and contamination. In order to establish training data suitable to train the classification model to reliably detect certain surface characteristic classes even after several years of influence of external factors such as heat and UV light from sunlight, exposure to salts, biological substances and other corrosive substances and contaminants, etc., an accelerated degradation of coat may be performed for some surfaces SU comprising a cured coat, in order to obtain surfaces with artificially degraded coat corresponding to various factors such as aging and/or staying in harsh environments.

Various accelerated lifetime test techniques exist to test the resistance of a coat to different influences, typically for development and documentation purposes. However, as these techniques serve to cause an accelerated degradation of the coat, they can be used herein to produce surfaces SU comprising cured coats that are artificially aged or otherwise degraded. Spectroscopy measurements ME taken on the artificially degraded surfaces SU may be used for training the classification model of the invention to detect surface characterizing classes of actually old and degraded surfaces comprising cured coats. In the sections below is described three methods of accelerated degradation of coatings, which can also be used in the context of the present invention to provide training measurement inputs for training classification of aged coats.

### QUV Accelerated Weathering

QUV, accelerated weathering, is used to simulate outdoor and indoor sun exposure on surface finishes and coatings, usually to test for weather resistance. The QUV test simulates the deterioration caused by sunlight, and water (e.g. rain and dew) by using fluorescent ultraviolet light (UV) and condensation. In a few days or weeks, the QUV tester can reproduce the damage that occurs over months or years outdoors. The artificial degradation may be performed by exposing the surface SU comprising a cured coat for 4 hours UV-light at 60° C. with Type 1A (UVA-340) lamps and 4 hours condensation at 50° C. Spectroscopy measurements ME may be acquired regularly during the process. QUV may be performed e.g. according to ISO 16474-3.

### Chemical Damaging

This is a method for attempting damaging a coating to the effect of chemicals by partial or full immersion, usually used for testing chemical resistance. In a long term approach, surfaces SU comprising cured coat are exposed to a liquid comprising chemicals that may occur in the natural environments of the intended use case for the respective coat, or chemicals with similar or stronger properties. Inspections, e.g. including acquisition of spectroscopy measurements ME by means of a surface characterizing device system SCD described above, may generally be performed after 7 days, 1 month and 3 months and with a final evaluation after 6 months. The surfaces SU are ⅔ immersed (unless otherwise agreed) and subject to storage temperatures of 23±2° C., if not otherwise specified. Long term test may for example be relevant for coating systems exposed to the same chemical for longer period e.g. storage tanks. In a medium term approach, the surfaces SU comprising a cured coat are exposed to a liquid as described above with inspections and spectroscopy measurements generally after 1 day, 3 days, 7 days and 1 month and with final evaluation after 3 months. The surfaces SU are ⅔ immersed (unless otherwise agreed) and subject to storage temperatures of 23±2° C., if not otherwise specified. Medium term test may for example be relevant for coating systems that will be exposed to a chemical for a limited period e.g. cargo tanks. A short term approach may be an aggressive simulation test of proprietary tank cleaning products and similar, where surfaces SU comprising a cured coat are exposed to a liquid as described above with inspections and spectroscopy measurements generally after 1 day, 3 days and with a final evaluation after 7 days. The panels are % immersed (unless otherwise agreed) and subjected to the temperature specified for the cleaning procedure for the tank cleaning product or 23±2° C., if nothing is specified. Chemical damaging may be performed e.g. according to ISO 2812-1.

### Thermal Degradation

Surfaces SU comprising a cured coat may be exposed to high heat to a point that the molecular structure of the coating system will alter. Spectroscopy measurements ME may be acquired regularly during the process. Thermal degradation may be performed e.g. according to ISO 14680.

### Mixed Data

Examples of spectroscopy measurements ME acquired by the surface characterising device SCD configured to perform ATR-based spectroscopy measurements, and distributed in a measurement device MD and a smart device SD, described above with reference to FIGS. 11-12, 21 and 25-26, are shown here in table form, together with their binder-system class labels, in Table 4 below. The table includes spectroscopy measurements that a mixed between measurements from relatively fresh coated surfaces, as well as spectroscopy measurements from surfaces that have undergone age accelerated degradation, measurements of coats of different colors, and measurements of coats of different products that fall under the same binder-system class labels.

TABLE 4
Examples of labelled spectroscopy measurements to use as training data
Table 4 - Labelled spectroscopy measurements including spectroscopy
measurements measured from coated surfaces that have undergone accelerated
age degradation. The table includes spectroscopy measurements from
relatively fresh coated surfaces, as well as spectroscopy measurements
from surfaces that have undergone age accelerated degradation.

| Binder- | | Wave- | Wave- | |
|---|---|---|---|---|
| system | | length | length | |
| class | ID | min. | max. | Measurement values |
| Epoxy | D94C95D1- | 5500 | 7940 | [9244, 8960, 9095, 9249, 9019, 9062, 9081, 9230, |
| Novolac | C621- | | | 9235, 9225, 9240, 9388, 9185, 9218, 9225, 9335, |
| | 4EB3- | | | 9278, 9229, 9230, 9371, 9232, 9335, 9231, 9328, |
| | 0EB2- | | | 9288, 9449, 9345, 9302, 9442, 9380, 9327, 9179, 9279, |
| | 08DB1BDCADB1 | | | 9355, 9395, 9268, 9267, 9321, 9209, 9230, 9283, |
| | | | | 9196, 9197, 9253, 9292, 9270, 9352, 9267, 9268, |
| | | | | 9294, 9290, 9255, 9260, 9261, 9274, 9168, 9187, 9282, |
| | | | | 9374, 9168, 9295, 9342, 9158, 9214, 9179, 9209, |
| | | | | 9193, 9277, 9104, 9276, 9105, 9170, 9058, 9015, |
| | | | | 9112, 9172, 9057, 9065, 9122, 8988, 9022, 9058, |
| | | | | 9000, 9076, 8935, 8908, 9012, 9004, 9045, 9008, |
| | | | | 8969, 8943, 9000, 9095, 9090, 9121, 9140, 9130, 9258, |
| | | | | 9265, 9372, 9206, 9214, 9301, 9326, 9189, 9350, |
| | | | | 9255, 9204, 9201, 9234, 9105, 9096, 8918, 8989, |
| | | | | 8799, 8878, 8788, 8698, 8673, 8584, 8515, 8698, |
| | | | | 8574, 8600, 8708, 8566, 8687, 8695, 8752, 8783, 8750, |
| | | | | 8875, 8737, 8849, 8996, 8887, 8925, 8936, 8951, |
| | | | | 8928, 8879, 8845, 8790, 8781, 8843, 8868, 8676, |
| | | | | 8652, 8647, 8630, 8656, 8614, 8596, 8547, 8489, |
| | | | | 8644, 8596, 8634, 8622, 8585, 8706, 8773, 8746, 8789, |
| | | | | 8822, 8907, 8820, 8775, 8770, 8857, 8865, 8669, |
| | | | | 8662, 8745, 8865, 8684, 8748, 8686, 8766, 8723, |
| | | | | 8807, 8778, 8745, 8828, 8744, 8655, 8886, 8822, |
| | | | | 8855, 8790, 8837, 8855, 8914, 8898, 8821, 8809, 8811, |
| | | | | 8882, 8864, 8864, 8994, 8692, 8910, 8888, 8825, |
| | | | | 8944, 8768, 8790, 8853, 8835, 8830, 8917, 8790, |
| | | | | 8846, 8672, 8738, 8819, 8625, 8626, 8628, 8600, |
| | | | | 8660, 8617, 8640, 8694, 8542, 8703, 8572, 8497, 8498, |
| | | | | 8596, 8630, 8605, 8502, 8589, 8611, 8528, 8572, |
| | | | | 8582, 8551, 8639, 8634, 8504, 8541] |
| Fouling | EB170A9A- | 5500 | 8000 | [9526, 9454, 9559, 9731, 9464, 9729, 9715, 9622, |
| Release | 563E- | | | 9532, 9764, 9722, 9588, 9512, 9617, 9587, 9650, |
| (Silicone) | 4741- | | | 9519, 9600, 9543, 9630, 9520, 9642, 9696, 9633, |
| | 26EF- | | | 9675, 9591, 9597, 9559, 9585, 9538, 9611, 9610, 9645, |
| | 08DB05D16207 | | | 9678, 9622, 9492, 9570, 9642, 9485, 9473, 9509, |
| | | | | 9626, 9602, 9483, 9687, 9592, 9495, 9574, 9478, |
| | | | | 9657, 9562, 9519, 9576, 9543, 9526, 9531, 9558, |
| | | | | 9484, 9590, 9568, 9587, 9582, 9603, 9500, 9412, 9658, |
| | | | | 9526, 9498, 9542, 9591, 9546, 9485, 9502, 9450, |
| | | | | 9515, 9459, 9613, 9452, 9502, 9423, 9469, 9599, |
| | | | | 9601, 9472, 9435, 9385, 9394, 9436, 9453, 9502, |
| | | | | 9423, 9445, 9478, 9463, 9470, 9477, 9532, 9517, 9483, |
| | | | | 9485, 9561, 9453, 9470, 9467, 9587, 9473, 9492, |
| | | | | 9452, 9637, 9521, 9449, 9430, 9493, 9523, 9582, |
| | | | | 9446, 9465, 9403, 9420, 9536, 9372, 9592, 9494, |
| | | | | 9392, 9373, 9460, 9553, 9392, 9404, 9556, 9459, 9438, |
| | | | | 9411, 9463, 9557, 9402, 9298, 9483, 9463, 9357, |
| | | | | 9396, 9391, 9216, 9329, 9305, 9452, 9374, 9294, |
| | | | | 9267, 9238, 9305, 9383, 9208, 9346, 9203, 9240, |
| | | | | 9223, 9225, 9283, 9162, 9233, 9269, 9231, 9216, 9173, |
| | | | | 9125, 9161, 9185, 9236, 9136, 9169, 9222, 9265, |
| | | | | 9149, 9276, 9143, 9179, 9306, 9194, 9349, 9236, |
| | | | | 9251, 9181, 9301, 9364, 9379, 9364, 9362, 9381, |
| | | | | 9358, 9440, 9390, 9414, 9299, 9351, 9443, 9386, 9347, |
| | | | | 9415, 9384, 9412, 9424, 9401, 9364, 9410, 9346, |
| | | | | 9371, 9326, 9337, 9366, 9459, 9272, 9472, 9342, |
| | | | | 9351, 9423, 9336, 9292, 9384, 9258, 9336, 9261, |
| | | | | 9225, 9303, 9283, 9208, 9260, 9280, 9134, 9119, 9134, |
| | | | | 9077, 9068, 8955, 8882, 8877, 8837, 8655, 8679, |
| | | | | 8488, 8464, 8353, 8162, 8056, 7915, 7792, 7683, |
| | | | | 7555, 7510, 7457, 7345] |
| Hard | F7340138- | 5500 | 8000 | [8390, 8403, 8336, 8412, 8396, 8380, 8454, 8345, |
| Antifouling | B348- | | | 8453, 8349, 8437, 8425, 8425, 8407, 8370, 8391, |
| | 4F78- | | | 8392, 8430, 8418, 8449, 8405, 8385, 8373, 8308, |
| | 2749- | | | 8363, 8364, 8381, 8310, 8358, 8310, 8322, 8300, 8293, |
| | 08DB05D16207 | | | 8311, 8269, 8268, 8266, 8273, 8300, 8247, 8275, |
| | | | | 8264, 8300, 8304, 8262, 8240, 8252, 8223, 8236, |
| | | | | 8238, 8175, 8200, 8175, 8131, 8143, 8173, 8154, |
| | | | | 8135, 8154, 8149, 8161, 8171, 8206, 8186, 8188, 8194, |
| | | | | 8190, 8216, 8167, 8161, 8197, 8142, 8134, 8155, |
| | | | | 8095, 8107, 8095, 8106, 8118, 8062, 8072, 8079, |
| | | | | 8009, 8084, 7996, 7991, 7986, 7994, 7965, 7957, |
| | | | | 7964, 7958, 7989, 7939, 7961, 7930, 7938, 7959, 7962, |
| | | | | 7977, 7936, 7935, 7967, 7957, 7973, 7984, 7947, |
| | | | | 7995, 7977, 7973, 7992, 7973, 7973, 7938, 7958, |
| | | | | 7935, 7927, 7952, 7977, 7958, 7961, 7976, 7946, |
| | | | | 7991, 7908, 7940, 7926, 7931, 7939, 7945, 7899, 7901, |
| | | | | 7918, 7928, 7919, 7987, 7901, 7937, 7957, 7903, |
| | | | | 7868, 7875, 7874, 7890, 7859, 7896, 7898, 7831, |
| | | | | 7891, 7873, 7882, 7854, 7835, 7853, 7813, 7829, |
| | | | | 7858, 7824, 7845, 7776, 7803, 7771, 7761, 7832, 7769, |
| | | | | 7742, 7739, 7774, 7755, 7689, 7770, 7699, 7702, |
| | | | | 7649, 7726, 7714, 7723, 7697, 7708, 7666, 7631, |
| | | | | 7654, 7658, 7709, 7654, 7696, 7691, 7638, 7690, |
| | | | | 7671, 7661, 7677, 7660, 7707, 7689, 7668, 7709, 7736, |
| | | | | 7716, 7685, 7692, 7736, 7726, 7686, 7664, 7740, |
| | | | | 7674, 7692, 7741, 7748, 7692, 7768, 7744, 7769, |
| | | | | 7722, 7720, 7744, 7735, 7720, 7742, 7766, 7727, |
| | | | | 7720, 7707, 7735, 7753, 7742, 7745, 7722, 7733, 7709, |
| | | | | 7732, 7736, 7741, 7769, 7774, 7723, 7753, 7740, |
| | | | | 7773, 7756, 7732, 7723, 7768, 7733, 7748, 7759, |
| | | | | 7750, 7734, 7732, 7729] |
| Alkyd | 1A16FF46- | 5500 | 8000 | [7226, 7514, 7639, 7494, 7497, 7475, 7641, 7373, |
| | B5AA- | | | 7472, 7545, 7509, 7357, 7445, 7296, 7233, 7171, |
| | 4CD1- | | | 7105, 7059, 6922, 6817, 6701, 6657, 6526, 6386, |
| | 270D- | | | 6192, 6089, 5930, 5784, 5713, 5559, 5423, 5377, 5242, |
| | 08DB05D16207 | | | 5127, 5083, 5035, 4979, 4928, 4906, 4928, 4978, |
| | | | | 5026, 5062, 5051, 5102, 5177, 5220, 5117, 5246, |
| | | | | 5308, 5419, 5387, 5471, 5449, 5497, 5577, 5587, |
| | | | | 5585, 5682, 5650, 5655, 5687, 5732, 5722, 5811, 5786, |
| | | | | 5774, 5835, 5820, 5867, 5841, 5893, 5835, 5932, |
| | | | | 5931, 5938, 5920, 5977, 5980, 5963, 5976, 6033, |
| | | | | 6067, 6138, 5992, 6121, 6136, 6165, 6222, 6271, |
| | | | | 6294, 6405, 6370, 6411, 6481, 6550, 6488, 6603, 6537, |
| | | | | 6660, 6651, 6690, 6745, 6734, 6798, 6845, 6792, |
| | | | | 6855, 6859, 6883, 6909, 6931, 6951, 6972, 6937, |
| | | | | 7000, 7021, 6917, 6907, 7009, 6990, 6767, 6951, |
| | | | | 6882, 6942, 6866, 6879, 6812, 6841, 6836, 6759, 6734, |
| | | | | 6705, 6668, 6609, 6636, 6599, 6552, 6580, 6572, |
| | | | | 6472, 6451, 6482, 6503, 6450, 6434, 6456, 6480, |
| | | | | 6449, 6399, 6427, 6447, 6390, 6477, 6491, 6400, |
| | | | | 6401, 6400, 6433, 6320, 6372, 6397, 6415, 6414, 6350, |
| | | | | 6414, 6340, 6307, 6331, 6329, 6312, 6419, 6342, |
| | | | | 6348, 6334, 6354, 6388, 6268, 6326, 6286, 6321, |
| | | | | 6328, 6371, 6364, 6353, 6346, 6383, 6398, 6418, |
| | | | | 6330, 6402, 6362, 6386, 6473, 6433, 6467, 6418, 6473, |
| | | | | 6485, 6401, 6459, 6465, 6401, 6472, 6456, 6411, |
| | | | | 6392, 6336, 6406, 6290, 6300, 6211, 6197, 6188, |
| | | | | 6092, 6135, 6023, 5992, 5967, 5901, 5842, 5813, |
| | | | | 5777, 5736, 5647, 5570, 5553, 5563, 5419, 5400, 5365, |
| | | | | 5290, 5201, 5154, 5121, 5062, 5041, 4960, 4965, |
| | | | | 4940, 4823, 4816, 4772, 4780, 4670, 4629, 4616, |
| | | | | 4568, 4584, 4527, 4536] |
| Epoxy | 92F1C89E- | 5000 | 8000 | [9285, 9075, 9265, 9277, 9200, 9214, 9260, 9313, |
| | 352D- | | | 9220, 9258, 9252, 9240, 9289, 9273, 9311, 9268, |
| | 4735- | | | 9293, 9291, 9283, 9250, 9336, 9271, 9291, 9314, |
| | 275A- | | | 9222, 9236, 9227, 9230, 9277, 9251, 9286, 9242, 9276, |
| | 08DB05D16207 | | | 9255, 9270, 9290, 9340, 9285, 9306, 9324, 9356, |
| | | | | 9320, 9330, 9308, 9295, 9303, 9278, 9237, 9299, |
| | | | | 9214, 9250, 9234, 9214, 9171, 9135, 9213, 9167, |
| | | | | 9143, 9197, 9177, 9212, 9196, 9224, 9188, 9240, 9181, |
| | | | | 9237, 9200, 9236, 9263, 9180, 9246, 9203, 9359, |
| | | | | 9240, 9209, 9211, 9197, 9222, 9252, 9181, 9208, |
| | | | | 9205, 9143, 9220, 9176, 9138, 9147, 9093, 9093, |
| | | | | 9195, 9174, 9142, 9130, 9133, 9184, 9159, 9151, 9200, |
| | | | | 9153, 9182, 9153, 9182, 9220, 9186, 9226, 9208, |
| | | | | 9219, 9174, 9170, 9186, 9116, 9176, 9176, 9161, |
| | | | | 9127, 9122, 9043, 9086, 9084, 9032, 9065, 9060, |
| | | | | 9059, 9002, 9065, 9074, 8992, 9048, 9027, 9065, 9070, |
| | | | | 9061, 9057, 9017, 9002, 8992, 9039, 9070, 9090, |
| | | | | 9011, 9025, 9020, 9105, 9056, 9066, 9117, 9133, |
| | | | | 9105, 9095, 9125, 9097, 9158, 9101, 9143, 9072, |
| | | | | 9119, 9136, 9135, 9130, 9112, 9147, 9110, 9105, 9022, |
| | | | | 9101, 9078, 9064, 9088, 9127, 9097, 9087, 9133, |
| | | | | 9139, 9084, 9055, 9102, 9094, 9083, 9126, 9143, |
| | | | | 9120, 9067, 9144, 9124, 9164, 9100, 9124, 9134, |
| | | | | 9192, 9144, 9334, 9149, 9107, 9173, 9185, 9190, 9170, |
| | | | | 9165, 9162, 9161, 9209, 9200, 9166, 9194, 9206, |
| | | | | 9139, 9175, 9202, 9166, 9125, 9152, 9150, 9120, |
| | | | | 9191, 9067, 9137, 9098, 9120, 9161, 9118, 9114, |
| | | | | 9158, 9139, 9117, 9093, 9079, 9069, 9030, 9073, 9097, |
| | | | | 9076, 9105, 9107, 9077, 9174, 9136, 9088, 9131, |
| | | | | 9088, 9118, 9098, 9164, 9103, 9082, 9166, 9072, |
| | | | | 9089, 9089, 9013, 9088] |
| Self- | C7184AE1- | 5500 | 8000 | [5809, 5729, 5914, 5972, 5975, 5837, 5847, 5980, |
| Polishing | 2854- | | | 5817, 5859, 5914, 5852, 5892, 5915, 5941, 5897, |
| Antifouling | 4656- | | | 5869, 5859, 5842, 5820, 5859, 5820, 5899, 5797, |
| | 272B- | | | 5765, 5832, 5810, 5860, 5831, 5842, 5708, 5704, 5763, |
| | 08DB05D16207 | | | 5696, 5640, 5605, 5564, 5542, 5543, 5553, 5486, |
| | | | | 5436, 5453, 5452, 5379, 5340, 5349, 5373, 5314, |
| | | | | 5315, 5382, 5358, 5333, 5381, 5346, 5328, 5382, |
| | | | | 5234, 5398, 5340, 5347, 5337, 5338, 5379, 5321, 5347, |
| | | | | 5341, 5393, 5406, 5329, 5295, 5313, 5261, 5323, |
| | | | | 5293, 5183, 5172, 5226, 5207, 5142, 5148, 5084, |
| | | | | 5049, 5125, 5145, 5030, 5079, 5016, 5031, 4969, |
| | | | | 5082, 5053, 5040, 5017, 5002, 5022, 4960, 4933, 4934, |
| | | | | 4952, 5022, 4958, 4969, 4983, 4942, 4967, 4955, |
| | | | | 4897, 4927, 4941, 4945, 4954, 4938, 4892, 4921, |
| | | | | 4957, 5123, 4928, 5029, 5019, 5029, 5002, 5010, |
| | | | | 4949, 4979, 5039, 5024, 5076, 5009, 5036, 5028, 5058, |
| | | | | 4939, 5010, 5022, 4989, 4970, 4758, 4488, 4338, |
| | | | | 4451, 4326, 4367, 4410, 4350, 4240, 4265, 4308, |
| | | | | 4285, 4213, 4279, 4252, 4252, 4135, 4185, 4160, |
| | | | | 4230, 4168, 4150, 4154, 4252, 4145, 4136, 4107, 4179, |
| | | | | 4087, 4075, 4168, 4101, 4048, 4022, 4028, 4097, |
| | | | | 4082, 3978, 4028, 3940, 4059, 4023, 3957, 4010, |
| | | | | 4003, 4001, 3974, 3944, 4012, 4019, 4054, 4828, |
| | | | | 4713, 4432, 4196, 4753, 4276, 4222, 4015, 4120, 4151, |
| | | | | 3948, 4095, 4310, 4356, 4188, 4168, 4136, 4148, |
| | | | | 4110, 4132, 4138, 4179, 4101, 4137, 4163, 4130, |
| | | | | 4148, 4188, 4162, 4115, 4122, 4188, 4148, 4105, |
| | | | | 4184, 4166, 4201, 4126, 4189, 4144, 4167, 4213, 4180, |
| | | | | 4181, 4250, 4196, 4151, 4219, 4169, 4158, 4208, |
| | | | | 4164, 4250, 4226, 4230, 4199, 4203, 4216, 4183, |
| | | | | 4203, 4216, 4220, 4164] |
| Acrylic | 59F659A7- | 5500 | 8000 | [8008, 8000, 8128, 8106, 8099, 8128, 8133, 8097, |
| | B1A5- | | | 8087, 8083, 8072, 8041, 7989, 8043, 8021, 8007, |
| | 4240- | | | 7885, 7783, 7748, 7619, 7499, 7369, 7254, 7090, |
| | 0F01- | | | 6897, 6724, 6605, 6394, 6213, 6051, 5898, 5689, 5558, |
| | 08DB1BDCADB1 | | | 5473, 5362, 5315, 5186, 5149, 5137, 5055, 5076, |
| | | | | 4965, 5003, 4968, 4991, 4993, 4987, 4999, 5104, |
| | | | | 5122, 5193, 5283, 5386, 5473, 5525, 5613, 5715, |
| | | | | 5820, 5872, 6013, 6100, 6188, 6294, 6330, 6454, 6498, |
| | | | | 6530, 6676, 6700, 6732, 6787, 6853, 6844, 6884, |
| | | | | 6962, 6953, 7004, 7021, 7033, 7036, 7009, 7035, |
| | | | | 7014, 6973, 6935, 6925, 6968, 6917, 6935, 6878, |
| | | | | 6825, 6827, 6802, 6819, 6738, 6722, 6679, 6642, 6631, |
| | | | | 6618, 6613, 6575, 6567, 6561, 6551, 6533, 6521, |
| | | | | 6522, 6498, 6485, 6530, 6588, 6557, 6575, 6566, |
| | | | | 6564, 6570, 6608, 6640, 6650, 6651, 6593, 6553, |
| | | | | 6530, 6496, 6492, 6393, 6316, 6277, 6157, 6119, 6018, |
| | | | | 5872, 5794, 5733, 5657, 5587, 5507, 5499, 5436, |
| | | | | 5408, 5410, 5360, 5368, 5425, 5391, 5459, 5480, |
| | | | | 5552, 5575, 5614, 5651, 5699, 5722, 5779, 5872, |
| | | | | 5904, 5973, 5977, 6081, 6081, 6100, 6131, 6256, 6226, |
| | | | | 6219, 6231, 6263, 6288, 6251, 6310, 6358, 6327, |
| | | | | 6300, 6283, 6313, 6304, 6298, 6310, 6298, 6382, |
| | | | | 6338, 6351, 6343, 6406, 6402, 6442, 6424, 6439, |
| | | | | 6475, 6524, 6554, 6538, 6564, 6531, 6557, 6557, 6567, |
| | | | | 6566, 6601, 6552, 6587, 6602, 6633, 6609, 6717, |
| | | | | 6634, 6645, 6660, 6618, 6628, 6631, 6617, 6692, |
| | | | | 6618, 6647, 6617, 6619, 6610, 6573, 6567, 6553, |
| | | | | 6524, 6536, 6473, 6441, 6463, 6395, 6379, 6369, 6332, |
| | | | | 6303, 6240, 6233, 6224, 6185, 6147, 6118, 6069, |
| | | | | 6031, 6005, 5990, 5943, 5908, 5933, 5883, 5866, |
| | | | | 5826, 5769, 5734, 5744] |
| Polysiloxane | 6CF8AC8B- | 5500 | 8000 | [8842, 8828, 8983, 8816, 8759, 8906, 8918, 8867, |
| | 58AE- | | | 8835, 8804, 8862, 8853, 8898, 8884, 8882, 8878, |
| | 458F- | | | 8825, 8918, 8841, 8843, 8845, 8754, 8767, 8829, |
| | 2930- | | | 8729, 8780, 8763, 8712, 8688, 8731, 8693, 8690, 8736, |
| | 08DB2EBA980B | | | 8738, 8666, 8671, 8679, 8687, 8700, 8719, 8710, |
| | | | | 8716, 8619, 8656, 8658, 8574, 8600, 8608, 8560, |
| | | | | 8566, 8517, 8562, 8490, 8498, 8518, 8520, 8493, |
| | | | | 8420, 8465, 8509, 8400, 8503, 8470, 8610, 8488, 8524, |
| | | | | 8542, 8571, 8517, 8534, 8585, 8524, 8556, 8546, |
| | | | | 8560, 8552, 8547, 8499, 8536, 8516, 8509, 8533, |
| | | | | 8493, 8518, 8522, 8468, 8494, 8526, 8520, 8510, |
| | | | | 8502, 8584, 8522, 8547, 8576, 8565, 8534, 8533, 8576, |
| | | | | 8623, 8630, 8577, 8442, 8597, 8637, 8595, 8616, |
| | | | | 8616, 8584, 8580, 8616, 8658, 8668, 8641, 8597, |
| | | | | 8591, 8576, 8533, 8568, 8470, 8513, 8505, 8537, |
| | | | | 8433, 8489, 8418, 8424, 8445, 8340, 8341, 8358, 8393, |
| | | | | 8320, 8303, 8275, 8244, 8251, 8153, 8178, 8179, |
| | | | | 8150, 8111, 8092, 8088, 8048, 8126, 8079, 8034, |
| | | | | 8058, 8066, 8009, 8000, 8043, 8040, 8020, 8022, |
| | | | | 7953, 8035, 8000, 8071, 8130, 8068, 8108, 8077, 8161, |
| | | | | 8132, 8145, 8082, 8168, 7914, 8159, 8086, 8117, |
| | | | | 8141, 8086, 8098, 8101, 8133, 8145, 8072, 8113, |
| | | | | 8146, 8087, 8139, 8205, 8159, 8127, 8132, 8125, |
| | | | | 8220, 8331, 8156, 8142, 8212, 8248, 8208, 8234, 8199, |
| | | | | 8245, 8207, 8220, 8186, 8246, 8223, 8265, 8257, |
| | | | | 8277, 8243, 8233, 8270, 8302, 8205, 8220, 8175, |
| | | | | 8277, 8225, 8228, 8180, 8223, 8248, 8205, 8176, |
| | | | | 8220, 8218, 8179, 8212, 8233, 8185, 8223, 8223, 8172, |
| | | | | 8172, 8165, 8106, 8079, 8065, 8027, 8003, 7946, |
| | | | | 7861, 7924, 7823, 7747, 7754, 7657, 7660, 7683, |
| | | | | 7647, 7630, 7614, 7604] |
| Polyurethane | 474C9722- | 5500 | 8000 | [8149, 8305, 8437, 8471, 8384, 8447, 8397, 8594, |
| | 6D42- | | | 8512, 8509, 8427, 8558, 8491, 8407, 8405, 8430, |
| | 45DE- | | | 8449, 8312, 8209, 8199, 8133, 8155, 8044, 8054, |
| | 0EF9- | | | 7766, 7684, 7561, 7525, 7372, 7269, 7078, 6955, 6783, |
| | 08DB1BDCADB1 | | | 6696, 6595, 6470, 6328, 6214, 6179, 6080, 5916, |
| | | | | 5872, 5771, 5696, 5660, 5449, 5465, 5548, 5533, |
| | | | | 5417, 5417, 5536, 5556, 5539, 5648, 5673, 5779, |
| | | | | 5791, 5961, 6007, 6088, 6170, 6258, 6295, 6365, 6489, |
| | | | | 6528, 6680, 6692, 6865, 6823, 6838, 6947, 6906, |
| | | | | 7009, 7087, 7050, 7065, 7209, 7266, 7131, 7193, |
| | | | | 7184, 7356, 7276, 7324, 7278, 7251, 7247, 7264, |
| | | | | 7258, 7294, 7237, 7280, 7215, 7193, 7099, 7065, 7170, |
| | | | | 7107, 7095, 7133, 7117, 7086, 7011, 6854, 6983, |
| | | | | 6932, 6881, 6840, 6910, 6917, 6867, 6825, 6723, |
| | | | | 6773, 6747, 6628, 6594, 6652, 6669, 6594, 6585, |
| | | | | 6507, 6417, 6405, 6327, 6294, 6174, 6138, 5973, 5846, |
| | | | | 5743, 5690, 5566, 5476, 5278, 5283, 5138, 4976, |
| | | | | 4968, 4972, 4862, 4779, 4790, 4696, 4744, 4773, |
| | | | | 4720, 4686, 4684, 4692, 4825, 4700, 4713, 4760, |
| | | | | 4724, 4750, 4756, 4826, 4856, 4980, 4961, 4939, 4996, |
| | | | | 5123, 5104, 5154, 5087, 5224, 5172, 5299, 5285, |
| | | | | 5354, 5376, 5417, 5389, 5336, 5425, 5464, 5490, |
| | | | | 5550, 5507, 5504, 5577, 5717, 5638, 5605, 5669, |
| | | | | 5814, 5791, 5814, 5883, 5900, 5871, 5910, 5891, 5987, |
| | | | | 5951, 5958, 5942, 6072, 5972, 5982, 6061, 6047, |
| | | | | 6027, 6085, 6103, 6080, 6181, 6179, 6199, 6249, |
| | | | | 6162, 6135, 6165, 6185, 6335, 6207, 6249, 6291, |
| | | | | 6275, 6217, 6200, 6284, 6243, 6256, 6219, 6325, 6272, |
| | | | | 6317, 6270, 6340, 6237, 6262, 6247, 6284, 6184, |
| | | | | 6185, 6115, 6160, 6167, 6157, 6117, 6006, 5985, |
| | | | | 5950, 5975, 5956, 5901] |
| Bad | 34D45413- | 5500 | 8000 | [10000, 10000, 10000, 10000, 10000, 10000, 10000, |
| scan | 5AAA- | | | 10000, 10000, 10000, 10000, 10000, 10000, 10000, |
| | 4B93- | | | 10000, 10000, 10000, 10000, 10000, 10000, 10000, |
| | 7B72- | | | 10000, 10000, 10000, 10000, 10000, 10000, 10000, |
| | 08DB2B8ED781 | | | 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, 10000, |
| | | | | 10000, 10000, 10000, 10000, 10000, 10000, |
| | | | | 10000, 10000, 10000, 10000, 10000, 10000, 10000, |
| | | | | 10000, 10000, 10000, 10000, 10000, 10000] |

FIGS. 27-29 illustrate examples of spectroscopy measurements ME acquired by the embodiment of a surface characterising device SCD with the magnetic attachment feature MSD, configured to perform ATR-based spectroscopy measurements as described above with reference to FIGS. 11-12, 21 and 25-26. All FIGS. 27-29 represent absorbance by a digital value on the vertical axis as a function of between 245 and 251 distributed samples on the horizontal axis. The samples represent wavelengths distributed over the range of wavelength from 5,500 nm to 8,000 nm. The spectroscopy measurement ME illustrated in FIG. 27 was performed on a surface SU comprising a cured coat belonging to the epoxy novolac binder class, and the spectroscopy measurements ME in FIGS. 28 and 29 were performed on surfaces SU comprising cured coats of the binder classes Polysiloxane and Acrylic, respectively, as indicated at the right side of the drawings.

FIG. 30 and FIG. 31 illustrate examples of user interfaces DUI of a smart device SD described above with reference to, among others, FIGS. 11 and 21. FIG. 30 illustrates a screenshot of a user interface DUI after performing measurement and classification of the spectroscopy measurement. The operator DO can see a measurement representation where the 250 measurement values have been smoothed, normalized and plotted as a graph, and the user interface also indicates the classification result, i.e. a determined surface characteristic class SCC, which in these two examples are binder classes. The binder class being “Polyuret” in this case. The user interface DUI further shows a date- and timestamp and the GPS coordinates, e.g., 55.7924, 12.5277, representing the location at which the measurements where acquired. Similarly, FIG. 31 illustrates a screenshot of the user interface DUI after performing a measurement and classification of a cured coat belonging to the Fouling release (Silicone) binder class.

The user interface DUI illustrated in FIGS. 30-31 may in various embodiments comprise more detail about the classification result, for example a percentage of classification certainty, or a list of alternative classes that came close. When the spectroscopy measurement ME is acquired with the purpose of training a classification model CM, the surface characterizing device system SCD and user interface DUI typically does not perform classification or show a classification result. Instead, the user interface may in various embodiments show more details about the spectroscopy measurement ME, such as any irregularities detected during measurement, e.g. not holding the device steady, not applying sufficient pressure, etc.

### Binder Classes

The classification model may be trained to classify, e.g., various different binder classes. The model may also be trained to classify the actual product. The following describes non-limiting examples of binder classes and associated products. Note that the following product names are trademarks of the Hempel company.

Polyurethane is an example of a binder class. Coatings of this binder class are typically prepared through the reaction of diisocyanates with polyols. The Hempel Hempathane HS with product number 55610 is an example of a product within the polyurethane binder class.

Epoxy is an example of a binder class. Epoxy coatings may typically refer to coatings prepared using glycidyl functional groups. They are typically crosslinked using amines, but other reactive groups such as thiols may be used. Also, homopolymerisation and reaction with alcohols can play a role. An example of a binder type used within this binder class is non-novolac epoxies. The Hempel Hempaprime Multi 500 with product number 45950 is an example of a product belonging to the epoxy binder class.

Self-polishing antifouling is a further example of a binder class. Self-polishing antifouling coatings may typically refer to cuprous oxide based antifouling coatings where a binder (typically physically drying) is formulated to self-polish. The coatings are characterised by a fairly low solids content, a high content of cuprous oxide (though copper free versions also exists) and a physically drying curing mechanism. Examples of binder types used withing this binder class is acrylic binders, rosin binders, vinyl copolymers, and also binders that are a mix of two or more of these. Hempel's AntiFouling Globic 9000 with product number 78950 is an example of a product belonging to the self-polishing antifouling binder class.

Alkyd is also an example of a binder class. Alkyd coatings are coatings typically prepared from (ao.) unsaturated fatty acids. The curing mechanism depends on the oxidation of the unsaturation of the fatty oil. To that purpose driers may be added. The binder type used in this binder class is alkyd. Hempalin Enamel with product number 52140 is an example of a product belonging to the alkyd binder class.

Fouling release (Silicone) is another example of a binder class. A fouling release coating is a coating intended to release fouling by having a low surface energy. Typically these coatings are prepared by using a polydimethylsiloxane (silicone)-based binder, and a silane as a curing agent. Hempaguard X7 with product number 89900 is an example of a product within the fouling release (silicone) binder class.

Acrylic is an example of a binder class. An acrylic binder class coating is a coating based on a polyacrylate backbone. The side chain may differ, but generally they are physically drying binders. Hemucryl with the product number 48191 is an example of a product withing the acrylic binder class.

Epoxy novolac is a further example of a binder class. An epoxy novolac is an epoxy coating based on a novolac type epoxy resin. Hence the binder type is a novolac epoxy. Hempadur with the product number 15500 is an example of a product belonging to the epoxy novolac binder class.

Polysiloxane is another binder class example. A polysiloxane-based coating is a coating based on a binder system that is partly of fully composed of polysiloxane-based binders. This may normally give good weatherability and gloss retention. The binder used in these coatings are polysiloxane based binders. Hempaxane Light with the product number 55030 is an example of a product belonging to the polysiloxane binder class.

Polyurea is a further example of a binder class. A polyurea based coating is composed of a diisocyanate binder cured with and amine-based curing agent. The binder type of this binder class is polyurea based. Hemparea DTM with the product number 55973 is an example of a product belonging to the polyurea binder class.

Hard antifouling is another example of a binder class. This coating of this binder class may also be known as ablative antifouling coatings. These coating types are normally based on acrylic binders containing significant amounts of soluble biocides. However, vinyl copolymer based binders and binders that are a mix of acrylic and vinyl copolymers may also be used as binder in these coatings. Hempel's AntiFouling Olympic+ with product number 72950 is an example of a product belonging to the hard antifouling binder type.

In the above description of the exemplified binder classes, Hempel products are mentioned as examples of actual coating products belonging to a given binder class. By labelling training data according to the product name (or product number), the classification model may thereby be trained to classify the actual product (or product number) of a coating. In this regard, it should be appreciated that by training the classification model on training data labelled with product name and/or product number of other coating manufacturers, the classification model may also be trained to classify these product names and/or product numbers from other manufacturers. Hence, the classification model is not limited to classify products from Hempel, but may in principle be trained to classify and/or identify products from any other manufacturer.

### Example 3

In a third example, the VGG16 classification model was trained and tested with regards to classifying surface characteristic classes based on spectroscopy measurements acquired from surfaces (training surfaces) coated with actual coating products comprising different binders, fillers, pigments etc., using the distributed surface characterizing device system SCD (see, e.g., the description referencing FIGS. 11-12, 21 and 25-26) implemented with single bounce ATR spectroscopy. In this particular example, the spectroscopy measurements were acquired from a mix of relatively fresh coated surfaces and coated surfaces that had been exposed to accelerated coat degradation, as described in the previous section. The applied accelerated coat degradation were made to mimic various different levels of degradation that occur to coatings withing an actual life expectancy of the coating. By including measurements from these degraded surfaces, the classification model is trained to classify surface characteristic classes even on degraded surfaces that have been exposed to harsh degrading conditions over several years of use. Thereby, the classification model may advantageously classify surface characteristic classes even more reliable and accurate when used on degraded surfaces, such as, e.g. age degraded surfaces compared to other models. In this example, the classification model were trained to classify binder classes including fouling release (silicone), self-polishing antifouling, epoxy, polyurethane, alkyd. Each binder class comprised examples of coatings with different color pigments and other different additives. As an example, the binder class epoxy included a number of examples with different colors and different product types. Advantageously, this may enable the classification model to distinguish between the binder classes without being biased by color pigment and other additives used in coatings.

Using the measurement device MD with the magnetic attachment feature MSD described in relation to FIGS. 11-12, spectroscopy measurements of 128 surfaces comprising a cured coat with a known binder class were acquired, as described in example 1. All the spectroscopy measurement were send from the measurement device to the cloud via an external buffer as, e.g., described in relation to FIGS. 11 and 14-16.

In a next step, the spectroscopy measurements were pre-processed and labeled according to the known binder class, as described in example 1, to produce labelled spectroscopy measurements. Two examples of the pre-processed plotted spectroscopy measurements are shown in FIGS. 30-31. Then, a random split were applied to establish a training data set comprising 64 labeled spectroscopy measurements and a test data set comprising 64 labelled spectroscopy measurements. Off note, the labelled spectroscopy measurements of the training data may sometimes be referenced as labelled training input measurements.

As in example 1, the VGG16 model was adopted as classification mode. The model was pretrained, trained and evaluated as described in example 1 above.

Table 5 below shows the confusion matrix generated based on the test data. The confusion matrix is a way of evaluating the model performance, and shows the classification of each test measurements of the test data set in relation to the actual true label associated with the test measurement. From table 5, it may, e.g., be appreciated that the trained classification model correctly classified 10 out of the 11 test measurements belonging to the binder class self-polishing antifouling, while only 1 of these where wrongly classified as epoxy.

TABLE 5
Confusion matrix

| | True surface characteristic class | | | | | |
|---|---|---|---|---|---|---|
| | | | | Fouling | | Self- |
| | | | | Release | | Polishing |
| | | Alkyd | Epoxy | (silicone) | Polyurethane | Antifouling |
| Classified | Alkyd | 7 | 1 | 1 | 0 | 0 |
| surface | Epoxy | 1 | 16 | 0 | 2 | 3 |
| characteristic | Fouling | 0 | 0 | 11 | 0 | 0 |
| classes | Release | | | | | |
| | (silicone) | | | | | |
| | Polyurethane | 0 | 2 | 0 | 8 | 1 |
| | Self- | 0 | 1 | 0 | 0 | 10 |
| | Polishing | | | | | |
| | Antifouling | | | | | |

Table 6 below shows the performance of the classification model as measured based on the test data set. From table 6, it may, e.g., be appreciated that the trained classification model had a recall of 100%, a precision of 92% and an F1 score of 96 for fouling release (silicone). In addition, the trained classification model had an overall accuracy of 81%.

TABLE 6

| Label | Recall | Precision | F1 | Images |
|---|---|---|---|---|
| Fouling | 100% | 92% | 96% | 11 |
| Release | | | | |
| (Silicone) | | | | |
| Self-Polishing | 91% | 71% | 80% | 11 |
| Antifouling | | | | |
| Epoxy | 73% | 80% | 76% | 22 |
| Polyurethane | 73% | 80% | 76% | 11 |
| Alkyd | 78% | 88% | 82% | 9 |
| Macro avg | 84% | 82% | 85% | 64 |

### Example 4

In a fourth example, the classification model was trained to classify actual product names of coatings of a coated surface. The procedure is similar to the procedure described in example 3, except that in this example the training data were labeled according to product name instead of according to binder class. This enables the classification model to classify the product name. The training data comprised spectroscopy measurements from surfaces coated with the following different products (all trademarks of the Hempel company): Hempathane HS, Hempaprime Multi 500, Hempel's AntiFouling Globic 9000, Hempalin Enamel, Hempaguard X7, Hemucryl, Hempadur, Hempaxane Light, Hemparea DTM, Hempel's AntiFouling Olympic+. The products represent various different binder classes and binder types.

### Fleet of Surface Characterizing Device Systems

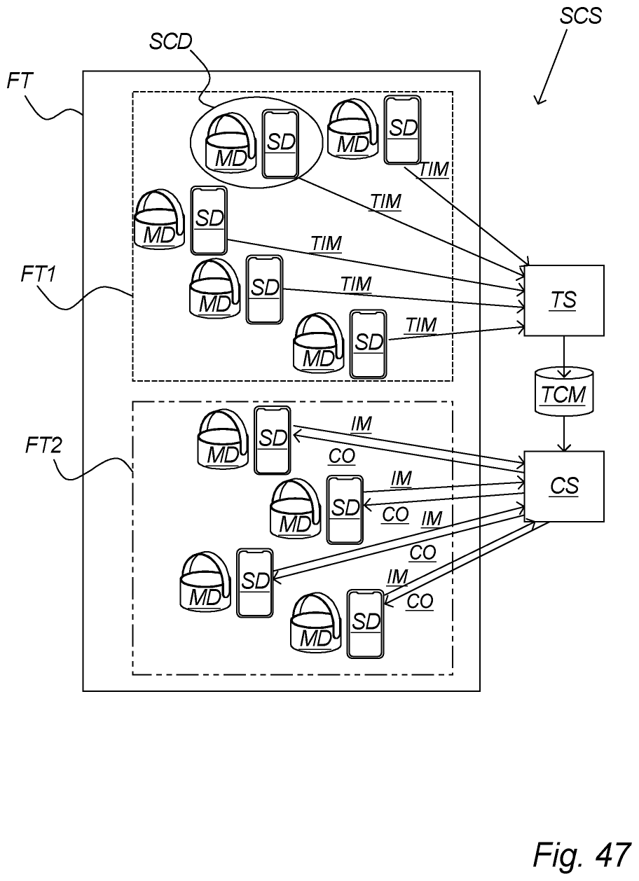

FIG. 47 illustrates a block diagram of a surface characterization system SCS according to an embodiment of the invention. The system comprises a fleet FT of surface characterizing device systems SCD, in this example each being distributed into a measurement device MD and a smart device SD. The description applies equally to an embodiment with single-device surface characterizing device systems SCD, or having a mix of distributed systems and single-device systems. Each of the surface characterizing device systems SCD are assigned to a first subset FT1 or a second subset FT2 of the fleet FT. The surface characterizing device systems SCD, i.e. each set of a measurement device MD and smart device SD, of the first subset FT1 are making measurements ME of surfaces comprising a cured coat, for example according to various embodiments as described herein. Representations of the measurements are transmitted as training input measurements TIM to a central training system TS. In other words, the training system TS receives training input measurements TIM from surface characterizing device systems SCD of the first subset FT1 of the fleet FT. The training system TS may use the training input measurements to train and test a classification model CM and produce a trained classification model TCM, as described later herein.

The surface characterizing device systems SCD of the second subset FT2 are also making measurements ME of surfaces comprising a cured coat, again for example as described elsewhere herein, for example by spectroscopy. The measurement technology used by surface characterization devices SCD of the first and second subsets FT1, FT2, may preferably be identical or similar. Representations of the measurements are used as input measurements IM to a classification system CS that classifies the surfaces based on the input measurements and returns classification outputs CO. In other words, the classification system CS receives input measurements IM from surface characterizing device systems SCD of the second subset FT2 of the fleet FT, and produces corresponding classification outputs CO using the trained classification model TCM trained by the training system TS, as described later herein.

The surface characterizing device systems SCD are preferably not fixed to a specific subset FT1, FT2, of the fleet FT, but may change roles over time. The role, i.e. subset, to which a surface characterizing device system SCD is assigned, may be decided or controlled based on capabilities of the surface characterizing device system, on the amount and quality of additional data about a measurement or a surface being measured, or for example on the experience and knowledge of an operator DO performing the measurement. The training system TS is preferably only using training input measurements TIM from surface characterizing device systems SCD that are belonging to the first subset FT1 at the time of performing the measurements on which the training input measurements TIM are based.

For example, in an embodiment only measurements and possibly associated metadata about the measurements, performed by experiences operators DO, are intended to be used as training data for training the classification model. Thus, a surface characterizing device system SCD may then be considered belonging to the first subset FT1 when operated by an experienced operator DO, e.g. determined by seniority, recommendations, past measurements, or another scoring system exceeding a predefined threshold, but only belonging to the second subset FT2 when operated by a less experienced operator below the threshold.

While shown together in the block diagram, the surface characterizing device systems SCD according to a preferred embodiment may be distributed geographically, even all over the world if relevant, and the orderly grouping in the drawing is not necessary in practical implementations, the devices from different subsets can be mixed geographically. The surface characterizing device systems SCD are also shown as identical devices in the drawing, but this is not required in preferred embodiments of the invention. Different versions, possibly with different capabilities, supplementary sensors, transporting and mounting options, different appearances e.g. due to external regulations, e.g. hazardous area requirements, or design preferences, may be implemented together in the same subsets FT1, FT2, of the fleet FT.

It may therefore not be possible to realize which subset FT1, FT2, a certain surface characterizing device system SCD belongs to simply from its appearance, location or operator, etc. A mechanism to ensure training is not performed on measurements from a second subset FT2 device may be implemented in the training system TS based on the content or quality of a received measurement, e.g. an associated device ID or operator ID, or be implemented in the surface characterizing device systems SCD so only devices from the first subset FT1 includes an option to transmit measurements to the training system TS, or includes a filtering of such an option based on the operator or available data about a measurement.

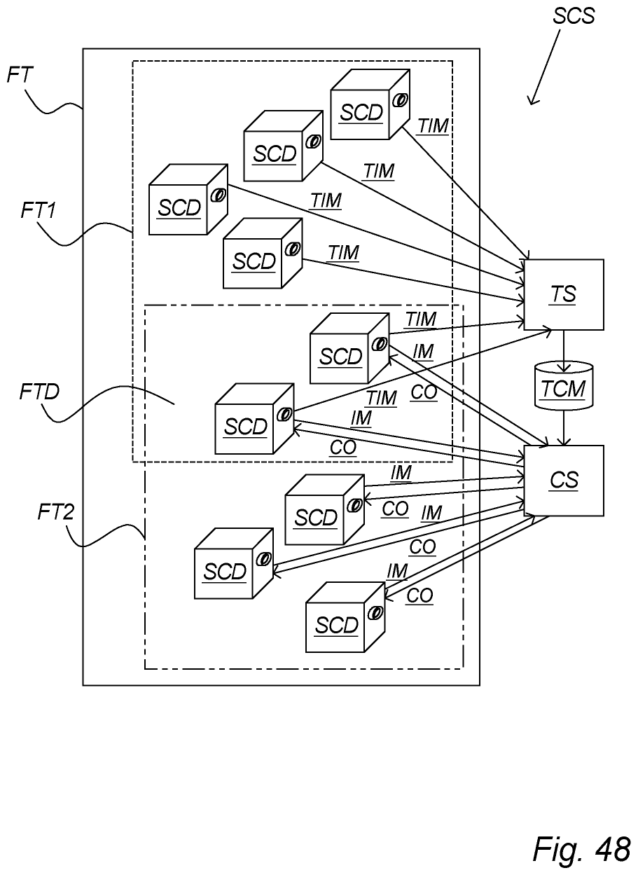

FIG. 48 illustrates in a block diagram another embodiment of a surface characterization system SCS according to the invention. For the sake of variation, this example is shown with single-device surface characterizing devices SCD, but as before, they can as well be implemented as distributed systems comprising a measurement device MD and smart device SD, the fleet FT can have a mix thereof. The system in FIG. 48 may comprise the fleet FT, first and second subsets FT1, FT, of surface characterizing device systems SCD, the central training system TS producing the trained classification model TCM, and the classification system for classifying surfaces comprising a cured coat, as described above with reference to FIG. 47.

Further, the embodiment illustrated in FIG. 48 comprises an overlap between the first subset FT1 and the second subset FT2 of the fleet FT, so that a number of dual role devices FTD are both transmitting training input measurements TIM to the training system TS and input measurements IM to the classification system CS. In an embodiment, the entire first subset FT1 belongs to the second subset FT2, whereby all surface characterizing device systems SCD can be used to perform or order a classification of a surface, but only some of the surface characterizing device systems also contribute to the training the of the classification model.

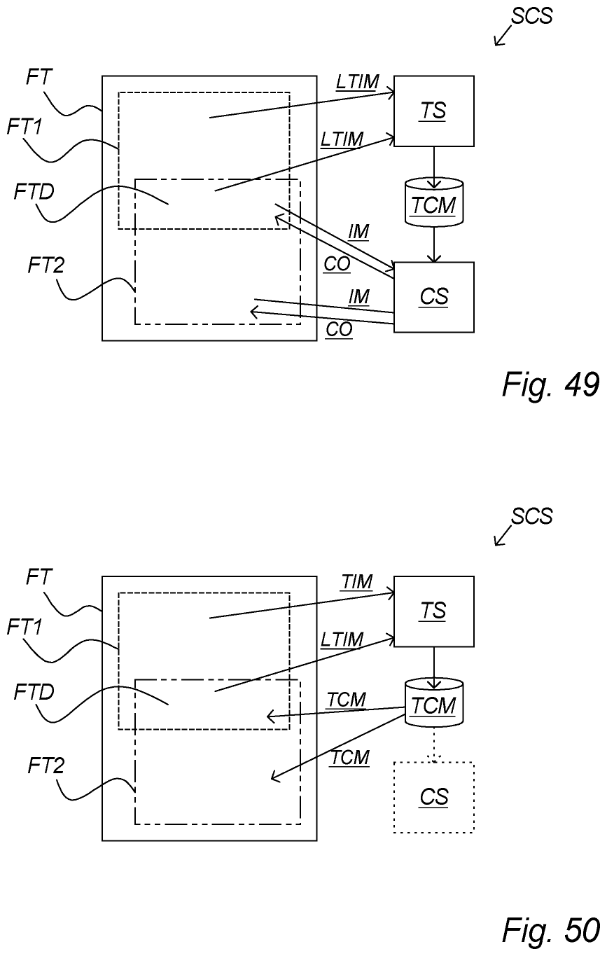

FIG. 49 illustrates in a block diagram another embodiment of a surface characterization system SCS according to the invention. The system in FIG. 49 may comprise the fleet FT, first and second subsets FT1, FT, of surface characterizing device systems SCD, the central training system TS producing the trained classification model TCM, and the classification system for classifying surfaces comprising a cured coat, as described above with reference to FIG. 47, and it may comprise dual role device FTD as described above with reference to FIG. 48.

Further, the embodiment of FIG. 49 illustrates that surface characterizing device systems SCD of the first subset FT1 of the fleet FT transmits labelled training input measurements LTIM to the training system TS. Labelled training input measurements LTIM comprises a training input measurement TIM and an associated training input label TIL designating one or more surface characteristic classes SCC, as described elsewhere herein. In this embodiment the training system TS may preferably be a supervised machine learning system, directly utilizing the training input labels TIL of the labelled training input measurements in the training process.

The establishment of labelled input training measurements LTIM, i.e. the association of training input labels TIL to surface measurements, may be achieved in various ways in the surface characterizing device systems SCD.

For example, a coated structure CTS or specific surface SU may be identified by a combination of a geolocation sensor such as GPS and a database of coated structure locations, or be identified using a tag reader for reading an ID by for example text recognition, bar code of RFID technology. The coated structure or surface may also be identified by input from the operator DO of the surface characterizing device system SCD. When a coated structure CTS or surface SU is identified, historical information about its coating, maintenance, environmental exposure, etc., may be accessible from a database, from which one or more matching surface characteristic classes SCC may be derived. Historical information may also comprise a very recent coating operation, and the database may for example comprise a log, ledger or journal, human memory, invoices, order confirmations, paint bucket labels, etc.

For example, a QR code, RFID/NFC tag or BLE beacon in a wind turbine blade or any other coated structure CTS may be read by a suitable supplementary sensor in the surface characterizing device system SCD, for example by a smart device SD thereof, such as smartphone or tablet computer carried by the operator DO, and the tag information itself may include a surface characteristic class SCC or include identification information to lookup the wind turbine blade in a database and retrieve a surface characteristic class SCC, which can be assigned to one or more measurements ME made on a surface SU of the coated structure CTS to provide a labelled training input measurement LTIM. For example, a geolocation sensor in the surface characterizing device system SCD, for example in a smart device SD thereof, such as smartphone or tablet computer carried by the operator DO, may provide the position where a measurement ME of a surface SU comprising a cured coat is performed, and a database lookup based on the geolocation may indicate which coated structure CTS comprises the measured surface SU. Suitable geolocation databases to indicate which coated structure is present at a certain location may comprise for example online world map applications or address directories, and for non-stationary coated structures such as ships or airplanes, the automatic identification system AIS or automatic dependent surveillance-broadcast ADB-S, may for example be used. The achieved identity of the coated structure may be looked up in a database and a surface characteristic class SCC retrieved, which can be assigned to one or more measurements ME made on a surface SU of the coated structure CTS to provide a labelled training input measurement LTIM.

In an embodiment, the operator DO manually assigns a surface characteristic class SCC to a measurement ME using the user interface DUI of the surface characterizing device system SCD, for example of a smart device SD thereof, such as a smartphone, tablet computer or laptop computer, based on his or her expert knowledge or assessment or information obtained in connection with the measurement, for example from visual inspection or locally held maintenance logs or knowledge. In an embodiment, the system is configured so that the operator DO is required to, or given the opportunity to, confirm any automatically assigned or suggested surface characteristic classes SCC before they are transmitted as labelled training input measurements LTIM.

In an embodiment, the training input measurements TIM are transmitted to the training system TS without surface characteristic classes SCC but preferably with intermediate information to facilitate assigning such classes SCC to produce a labelled training input measurement LTIM in the central training system TS, for example geolocation, identity of the coated structure CTS or specific surface SU, information from maintenance logs, information from tags on the surface, and/or the operator's qualified estimate.

The probability of a derived surface characteristic class SCC to actually belong to the surface SU that has been measured, e.g. reduced in case of several possible surfaces SU near a GPS location, or the probability that the surface characteristic class SCC derivable from historical information or human knowledge is correct, e.g. reduced with the age of the records or unexperienced operator, may be transmitted together with the labelled training input measurement LTIM for the training system TS to be able to discard training input measurements TIM where the assigned training input label TIL is below a probability threshold, e.g. below 95%, 90% or 80% probability for being correctly assigned. The discarding for being below a predefined probability threshold may also be performed at the surface characterizing device system SCD or by the operator DO. When a probability of an assigned training input label TIL is below the threshold, or no training input label TIL can be assigned at all with respect to a certain measurement ME, the respective surface characterizing device system SCD is removed from the first subset FT1 for the respective measurement, so that the measurement is not allowed to contribute to the training of the classification model. The respective measurement and surface characterizing device system SCD may still belong to the second subset FT2.

In an alternative embodiment, the assigning of training input labels TIL to the training input measurements received from the first subset FT2 may be performed in the central training system TS based on information established in the training system or received from e.g. external systems, e.g. maintenance databases.

FIG. 50 illustrates in a block diagram another embodiment of a surface characterization system SCS according to the invention. The system in FIG. 50 may comprise the fleet FT, first and second subsets FT1, FT, of surface characterizing device systems SCD, the central training system TS producing the trained classification model TCM, and the classification system for classifying surfaces comprising a cured coat, as described above with reference to FIG. 47, and it may comprise dual role device FTD as described above with reference to FIG. 48. The surface characterizing device systems SCD of the first subset FT1 may transmit training input measurements TIM and/or labelled training input measurements LTIM to the central training system TS as described above.

Further, the embodiment of FIG. 50 illustrates that surface characterizing device systems SCD of the second subset FT2 of the fleet FT receives the trained classification model TCM instead or, or in addition to, exchanging input measurements IM and classification outputs CO with a central classification system CS. In this embodiment, a classification system is thereby distributed among at least some of the surface characterizing device systems SCD of the second subset FT2. The trained classification model TCM that is downloaded to surface characterizing device systems SCD does preferably not have to be updated very often, and updating once every one or two weeks, once a month or 1-4 times a year, may provide sufficiently reliable classification results. The distribution of the trained classification model TCM enables classification of surfaces SU comprising a cured coat directly after performing a measurement ME, even when there is no access to a central classification system CS, e.g. due to lack of network coverage onsite. In an embodiment, a classification may be performed locally by means of a downloaded trained classification model TCM, while also transmitting an input measurement IM to a central classification system CS to perform a classification there, for example based on a more complex and resource demanding trained classification model TCM or to ensure benefit of the most recent updates. In an embodiment, a classification may be performed locally by means of a downloaded trained classification model TCM, while also transmitting the measurement as a training input measurements TIM to the training system TS for assisting in improving the trained classification model TCM. A training input label TIL may be assigned to the trained input measurement TIM, for example based on confirming the correctness of a locally acquired classification output CO from the downloaded trained classification model TCM.

In an embodiment the surface characterization system SCS may thereby be configured so that the first subset FT1 of surface characterizing device systems SCD is commissioned to improve the utilization of the second subset FT2 of surface characterizing device systems SCD to directly, or in combination with a central classification system CS, classify surfaces SU comprising a cured coat into predefined surface characteristic classes SCC. They may be causing this improvement by providing training input measurements TIM usable by the central training system TS to improve the trained classification model TCM.

It is noted that for all of the embodiments described above, the exchange of training input measurements TIM, labelled training input measurements LTIM, input measurements IM, classification outputs CO and trained classification models TCM, may be conducted between the surface characterizing device systems SCD and the central training system TS or classification system CS, for example by means of communication channels DCC established from communication modules DCM in the surface characterizing device systems, and preferably via the Internet, for example in a single-device surface characterizing device system or through a smart device SD of a distributed system, for example a smartphone, tablet computer, laptop computer, etc. and preferably via the Internet. Likewise, the processing of measurements, the assigning or confirmation of training input labels TIL or surface characteristic classes SCC, or classification using a downloaded trained classification model TCM, may be performed using a processor DP and memory DM of a single-device surface characteristic device SCD, or by distributing these tasks to a smart device SD or measurement device MD thereof.

### CLAUSES

The invention may also be described by the following system of clauses:
A1. A surface characterizing device system (SCD), comprising
   at least one sensor (DS) configured to acquire a measurement (ME) of at least one characteristic of a surface (SU) comprising a cured coat;
   a processor (DP) and memory (DM) configured to establish a representation (MER) of said measurement (ME);
   a visual output device (DD), such as an LED or display, configured to indicate at least a measurement or connection status;
   a user interface (DUI) configured to control measurement; and
   a communication module (DCM) configured to establish a communication channel (DCC) with a cloud (DC) and transmit said representation (MER) of said measurement (ME) via said communication channel (DCC);
   wherein said surface characterizing device system (SCD) is a portable system and being battery powered;
   wherein said surface characterizing device system (SCD) comprises a measurement buffer (DMB) and is configured to temporarily store said representation (MER) of said measurement (ME) in said measurement buffer (DMB) when said communication module (DCM) is prevented from establishing said communication channel (DCC) and transmit said temporarily stored representation (MER) upon reestablishment of said communication channel (DCC).
A2. The surface characterizing device system (SCD) of clause A1, wherein said temporarily stored representation (MER) is a training input measurement (TIM) or a labelled training input measurement (LTIM) for a training system (TS) configured to train a classification model (CM) for characterizing a surface comprising a cured coat.
A3. The surface characterizing device system (SCD) of clause A1 or A2, wherein said temporarily stored representation (MER) is an input measurement (IM) for a classification system (TS) configured to classify said surface (SU) comprising a cured coat.
A4. The surface characterizing device system (SCD) of any of the preceding clauses, wherein the surface characterizing device system (SCD) is a distributed system comprising a portable measurement device (MD) and portable smart device (SD), such as a smart phone, tablet computer or laptop computer;
wherein the measurement device (MD) comprises at least one of said at least one sensor (DS);
wherein the smart device (SD) comprises at least said communication module (DCM) configured to establish said communication channel (DCC) with said cloud (DC); and
wherein the measurement device (MD) is communicatively coupled to said smart device (SD), such as by Bluetooth, Wi-Fi, or a USB-cable, to transfer said measurement (ME) or said representation (MER).
A5. The surface characterizing device system (SCD) of clause A4, wherein the measurement buffer (DMB) is comprised in the smart device (SD); and wherein the user interface (DUI), visual output device (DD), processor (DP) and memory (DM), respectively, are comprised in the smart device (SD) and/or in the measurement device (MD).
A6. The surface characterizing device system (SCD) of clause A4 or A5, wherein the processor (DP) and memory (DM) configured to establish said representation (MER) to be transmitted via said communication channel (DCC) are comprised in the smart device (SD), and wherein the measurement device (SD) is configured to transfer said measurement (ME) to said smart device (SD) via said communicative coupling.
A7. The surface characterizing device system (SCD) of any of the clauses A4-A6, wherein the smart device (SD) comprises said visual output device (DD) and said user interface (DUI), such as combined in a touch screen.
A8. The surface characterizing device system (SCD) of any of the clauses A4-A7, wherein the visual output device (DD) and/or the user interface (DUI), respectively, are distributed between the measurement device (MD) and the smart device (SD), such as one or more status LEDs and buttons on the measurement device (MD) and a touch screen on the smart device (SD).
A9. The surface characterizing device system (SCD) of any of the clauses A4-A8, wherein the at least one sensor (DS) is at least two sensors (DS) and wherein said smart device (SD) comprises at least one of said at least two sensors (DS).
A10. The surface characterizing device system (SCD) of any of the clauses A4-A9, wherein the smart device (SD) is configured to assign to the representation (MER) of said measurement (ME) further data from a sensor (DS) of said smart device (SD) and/or training input labels (TIL) and/or metadata obtained by the smart device (SD), for example relating to geolocation, weather, temperature, humidity, light, operator (DO), surface (SU), asset type, asset identification, etc., or information about periods where said communication module has been prevented from establishing said communication channel.
A11. The surface characterizing device system (SCD) of any of the preceding clauses, wherein the surface characterizing device system (SCD) is configured to perform metadata synchronization between the time and/or geolocation of said measurement (ME) and any metadata included in the representation (MER) being transmitted via said communication channel (DCC) after said temporary storing in said measurement buffer (DMB).
A12. The surface characterizing device system (SCD) of any of the preceding clauses, wherein said measurement buffer (DMB) is configured to temporarily store at least two, such as at least 5, for example at least 10, 25 or 50, of said representations (MER) of said measurements (ME).
A13. The surface characterizing device system (SCD) of any of the preceding clauses, wherein said measurement buffer (DMB) is configured to delete said temporarily stored representations (MER) of said measurements (ME) when they have been transmitted upon reestablishment of said communication channel (DCC) and/or upon request from an operator (DO) of said surface characterizing device system (SCD).
A14. The surface characterizing device system (SCD) of any of the preceding clauses, wherein said surface characterizing device system (SCD) is configured to indicate by said visual output device (DD) a status of said measurement buffer (DMB), such as a number of temporarily stored representations (MER), a number of remaining free storage positions, an indication that the buffer is full, or whether the measurement buffer (DMB) is active due to establishment of said communication channel being prevented.
A15. The surface characterizing device system (SCD) of any of the preceding clauses, wherein said surface characterizing device system (SCD) is configured to perform pre-processing of said representation (MER) before said temporarily storing in said measurement buffer (DMB).
A16. The surface characterizing device system (SCD) of any of the preceding clauses, wherein said surface characterizing device system (SCD) is configured to encrypt and/or compress said representation (MER) before said temporarily storing in said measurement buffer (DMB), or when transmitting said temporarily stored representations (MER) via said communication channel (DCC).
A17. The surface characterizing device system (SCD) of any of the preceding clauses, wherein the communication module (DCM) is a wireless communication module, such as a Wi-Fi, GSM, LTE, 4G or 5G communication module.
A18. The surface characterizing device system (SCD) of any of the preceding clauses, wherein said sensor (DS) is configured for making spectroscopy measurements (ME).
A19. The surface characterizing device system (SCD) of clause A18, wherein the sensor (DS) is configured to obtain the spectroscopy measurements (ME) by attenuated total reflectance ATR spectroscopy at wavelengths in the range of 5,500 nm to 8,000 nm.
A20. The surface characterizing device system (SCD) of any of the preceding clauses, wherein said surface characterizing device system (SCD) comprises at least one infrared emitter (DE), at least one prism (DPR) and at least one sensor (DS) configured for attenuated total reflection spectroscopy of said surface (SU) comprising a cured coat to obtain a spectroscopy measurement (ME), such as single-bounce attenuated total reflection spectroscopy.
A21. The surface characterizing device system (SCD) of any of the preceding clauses, wherein said at least one infrared emitter (DE), said at least one prism (DPR) and said at least one sensor (DS) are configured to perform spectroscopy in the long wavelength infrared LWIR range, such as in the range of 8,000 nm to 10,500 nm, in the medium wavelength infrared MWIR range, such as in the range of 5,500 nm to 8,000 nm, or a combination thereof, such as the range of 5,500 nm to 10,500 nm.
A22. The surface characterizing device system (SCD) of any of the preceding clauses, wherein said sensor (DS) is a tuneable pyroelectric detector configured to obtain measurements at different wavelengths.
A23. The surface characterizing device system (SCD) of any of the preceding clauses, wherein said surface characterizing device system (SCD) comprises a multispectral or hyperspectral imaging camera (DHIC) for performing spectroscopy of said surface (SU) comprising a cured coat to obtain a spectroscopy measurement (ME), preferably in the near infrared NIR range, such as in the range of 930 nm to 2500 nm, or the mid infrared MIR range.
A24. The surface characterizing device system (SCD) of any of the preceding clauses, wherein said surface characterizing device system (SCD) comprises a pressure sensor (DPS) and/or an accelerometer sensor (DAS).
A25. The surface characterizing device system (SCD) of any of the preceding clauses, wherein said surface characterizing device system (SCD) comprises pressure maintenance means, e.g. magnetic or suction means.
A26. The surface characterizing device system (SCD) of any of the preceding clauses, wherein said surface characterizing device system (SCD), such as a measurement device (MD) thereof, comprises a magnetic switchable device (MSD) configured to releasable maintain attachment to said surface (SU).
A27. The surface characterizing device system (SCD) of any of the preceding clauses, wherein said surface characterizing device system (SCD) comprises a spacer arranged to maintain a predefined distance to said surface comprising a cured coat during said spectroscopy measurement.
A28. The surface characterizing device system (SCD) of any of the preceding clauses, wherein said sensor (DS) comprises an ultrasound transmitter and an ultrasound receiver.
A29. The surface characterizing device system (SCD) of any of the preceding clauses, wherein said sensor (DS) comprises a camera sensor (DCS), such as a visible spectrum camera, and optionally a flash light (DFL).
A30. The surface characterizing device system (SCD) of any of the preceding clauses, wherein said surface characterizing device system (SCD) comprises one or more of an infrared flash light (DIFL), a light sensor (DLS), a temperature sensor (DTS), a humidity sensor, a geolocation means such as GPS, a user interface (DUI), a visual output device (DD).
A31. The surface characterizing device system (SCD) of any of the preceding clauses, wherein the surface characterizing device system (SCD) is handheld, and preferably at least the measurement device (MD) comprises handles (DH).
A32. The surface characterizing device system (SCD) of any of the preceding clauses, wherein the surface characterizing device system (SCD) or at least the measurement device (MD) is transported by a robot or drone (DR).
A33. The surface characterizing device system (SCD) of any of the preceding clauses, wherein said surface characterizing device system (SCD) is configured to perform a filtering or pre-validation of measurements (ME) and determine whether to temporarily store said representation (MER) of said measurement (ME) in said measurement buffer (DMB) based on a result of said filtering or pre-validation.
A34. The surface characterizing device system (SCD) of any of the preceding clauses, wherein said surface characterizing device system (SCD) is configured to discard measurements (ME) that are not suitable for training or classification.
A35. The surface characterizing device system (SCD) of any of the preceding clauses, wherein said surface characterizing device system (SCD) is configured to indicate by said visual output device (DD) a result of said filtering or pre-validation or whether a measurement is suitable.
A36. The surface characterizing device system (SCD) of any of the preceding clauses, wherein said surface characterizing device system (SCD) comprises a trained classification model (TCM) for characterizing a surface comprising a cured coat, and wherein said surface characterizing device system (SCD) is configured to perform a classification based on said representation (MER) of said measurement (ME) and determine whether to temporarily store said representation (MER) on the basis of a result of said classification.
A37. The surface characterizing device system (SCD) of clause A36, wherein said trained classification model (TCM) is configured to classify said surface (SU) into at least one predefined surface characteristic class (SCC) from a plurality of predefined surface characteristic classes (SCC) based on said measurement (ME), the surface characteristic classes (SCC) comprising one or more classes selected from a group of binder system classes, a group of coat manufacturer classes, a group of coating product classes, or a combination thereof.
A38. The surface characterizing device system (SCD) of any of the preceding clauses, wherein said surface characterizing device system (SCD) is configured to download an updated trained classification model (TCM) upon reestablishment of said communication channel (DCC), optionally according to a time schedule.
A39. The surface characterizing device system (SCD) of any of the preceding clauses, wherein said surface characterizing device system (SCD) is configured to transmit a classification output (CO) from a trained classification model (TCM) stored in said surface characterizing device system, preferably together with a classification model version number, and wherein said classification output (CO) is temporarily stored in said measurement buffer (DMB) when the communication module (DCM) is prevented from establishing said communication channel (DCC).
A40. The surface characterizing device system (SCD) of any of the preceding clauses, wherein the surface (SU) is a surface of a coated structure (CST) preferably selected from the list of a ship hull, a tank interior, e.g. the inside of a ballast tank, a wind turbine tower, a wind turbine blade, a bridge, an oil rig, a building, a chimney or an industrial
A41. The surface characterizing device system (SCD) of any of the preceding clauses, wherein the surface (SU) is a surface of a coated structure (CST) comprising a base structure (BSS) preferably of metal, such as steel, iron or aluminium, concrete, composites, such as reinforced composites e.g. glass fibre.
A42. The surface characterizing device system (SCD) of any of the preceding clauses, wherein the method comprises cleaning said surface (SU) comprising a cured coat before establishing said measurement (ME).
A43. The surface characterizing device system (SCD) of any of the preceding clauses, wherein said surface characterizing device system (SCD) is configured for use in electromagnetic shielding or weakening enclosures, for example steel enclosures such as tanks or cargo holds; concrete structures such as buildings, silos or basements.
A44. Method of measuring a characteristic of a surface (SU) comprising a cured coat; the method comprising steps of:
   providing a surface characterizing device system (SCD) in a first environment where communication with a cloud (DC) external to said first environment is prevented;
   using the surface characterizing device system (SCD) to acquire a measurement (ME) of at least one characteristic of a surface (SU) comprising a cured coat;
   storing a representation (MER) of said measurement (ME) temporarily in a measurement buffer (DMB) of said surface characterizing device system (SCD);
   providing said surface characterizing device system (SCD) in a second environment;
   establishing a communication channel (DCC) between said surface characterizing device system (SCD) and said cloud (DC); and
   transmitting said temporarily stored representation (MER) of said measurement (ME) to said cloud (DC).
A45. The method of clause A44, wherein said method is implemented by a surface characterizing device system (SCD) according to any of the clauses A1-A43.
A46. A system comprising the surface characterizing device system (SCD) of any of clauses A1-A43 and an unmanned vehicle (DR) such as a drone or robot, wherein the surface characterizing device system (SCD), or at least a measurement device (MD) thereof, is mounted on or integrated in the unmanned vehicle (DR).
A47. Computer-implemented training method for training a classification model for characterizing a surface comprising a cured coat; the training method comprising steps of:
receiving training input measurements (TIM) from a surface characterizing device system (SCD) according to any of the clauses A1-A43 based on measurements (ME) of training surfaces (TSU) comprising a cured coat;
generating labelled training input measurements (LTIM) by individually labelling said training input measurements (TIM) in accordance with a plurality of predefined surface characteristic classes (SCC);
establishing a training data set (TDS) on the basis of said labelled training input measurements (LTIM);
providing a classification model (CM);
training said classification model (CM) based on said training data set (TDS) to provide a trained classification model (TCM).
A48. Computer-implemented classification method of characterizing a surface (SU) comprising a cured coat, the method comprising steps of:
providing a trained classification model (TCM) by the training method of clause A47;
receiving an input measurement (IM) from a surface characterizing device system (SCD) according to any of the clauses A1-A43 based on a measurement (ME) of said surface (SU) comprising a cured coat; and
classifying said surface (SU) into at least one of said predefined surface characteristic classes (SCC) based on said input measurement (IM) using said trained classification model (TCM) to produce a classification output (CO).
A49. A training system (TS) configured to train a classification model (CM) for characterizing a surface comprising a cured coat; the training system (TS) comprising:
a training input measurement receiver (TIMR) configured to receive training input measurements (TIM) from a surface characterizing device system (SCD) according to any of the clauses A1-A43 based on measurements (ME) of training surfaces (TSU) comprising a cured coat;
a training input measurement labeller (TIML) configured to generate labelled training input measurements (LTIM) by individually labelling said training input measurements (TIM) in accordance with a plurality of predefined surface characteristic classes (SCC);
a training data set generator (TDSG) configured to generate a training data set (TDS) on the basis of said labelled training input measurements (LTIM);
a classification model (CM);
a training module (TM) configured to train said classification model (CM) based on said training data set (TDS) to provide a trained classification model (TCM).
A50. A trained classification model (TCM) for characterizing a surface (SU) comprising a cured coat; the trained classification model (TCM) being established by the training method of clause A47 or the training system (TS) of clause A49.
A51. A classification system (CS) configured to characterize a surface (SU) comprising a cured coat, the classification system (CS) comprising:
a trained classification model (TCM) according to clause A50 or provided according to the training method of clause A47 or provided by the training system (TS) of clause A49;
an input measurement receiver (IMR) configured receive an input measurement (IM) from a surface characterizing device system (SCD) according to any of the clauses A1-A43 based on a measurement (ME) of said surface (SU) comprising a cured coat; a classifier (C) configured to classify said surface (SU) into at least one of said predefined surface characteristic classes (SCC) based on said input measurement (IM) using said trained classification model (TCM) to produce a classification output (CO).
A52. Computer-implemented classification method of characterizing a surface (SU) comprising a cured coat, the method comprising steps of:
receiving an input measurement (IM) from a surface characterizing device system (SCD) according to any of the clauses A1-A43 based on a measurement (ME) of said surface (SU) comprising a cured coat; and
classifying said surface (SU) into at least one of a plurality of predefined surface characteristic classes (SCC) based on said input measurement (IM) using a trained classification model (TCM) according to clause A50 or provided according to the training method of clause A47 or provided by the training system (TS) of clause A49 to produce a classification output (CO).
A53. Use of the classification method of clause A48 or A52, or the classification system (CS) of clause A51 to identify, for a surface (SU) comprising a cured coat, one or more of a coat binder system, a coat manufacturer, a coat product, or to determine whether a coating system is compatible with said surface (SU) comprising a cured coat, or to determine whether overcoating of said surface (SU) comprising a cured coat is due.
A54. A training data set (TDS) for use in the training method of any of clause A47 or the training system (TS) of clause A49, comprising labelled training input measurements (LTIM) based on measurements (ME) of training surfaces (TSU) and labelled in accordance with a plurality of predefined surface characteristic classes (SCC).
A55. The training data set of clause A54, wherein the training data set comprises at least 10, such as at least 20, preferably at least 40, labelled training input measurements (LTIM) for each of said predefined surface characteristic classes (SCC).
A56. The training data set of clause A54 or A55, wherein each of said labelled training input measurements (LTIM) comprises an array of values based on absorbance or reflectance measured at different wavelengths on one of said training surfaces (TSU) and an associated training input label (TIL) designating one or more of said predefined surface characteristic classes (SCC).
A57. The training data set of any of clauses A54-A56, wherein each of said labelled training input measurements (LTIM) comprises an image generated from absorbance or reflectance measurements at different wavelengths on one of said training surfaces (TSU) and an associated training input label (TIL) designating one or of said predefined surface characteristic classes (SCC).
A58. The device system, method of measuring, system, training method or system, classification method or system, model, use or training data set of any of the preceding clauses, wherein said plurality of predefined surface characteristic classes (SCC) comprises one or more classes selected from a group of binder system classes, a group of coat manufacturer classes, a group of coating product classes, or a combination thereof.
A59. The device system, method of measuring, system, training method or system, classification method or system, model, use or training data set of any of the preceding clauses, wherein said plurality of predefined surface characteristic classes (SCC) is a plurality of classes selected from the group of binder system classes.
A60. The device system, method of measuring, system, training method or system, classification method or system, model, use or training data set of clause A58 or A59, wherein said group of binder system classes comprises one or more class from the list of acrylic, epoxy (including for example novolac epoxy and non-novolac epoxy), polyaspartic, polyurethane, polysiloxane, alkyd, silicate, silicone, polyurea, rosin, vinyl copolymers, polydimethylsiloxane, and hybrid technologies like for example epoxy/acrylic, epoxy/siloxane and epoxy/silicates binder classes, preferably at least one or more of the classes of epoxy, polyurethane and alkyd binder system classes.
A61. The device system, method of measuring, system, training method or system, classification method or system, model, use or training data set of any of the preceding clauses, wherein said predefined surface characteristic classes (SCC) comprises a class for unknown outcomes and/or unsuitable input measurements.
A62. The device system, method of measuring, system, training method or system, classification method or system, model, use or training data set of any of the preceding clauses, wherein said predefined surface characteristic classes (SCC) do not comprise pigment classes or color classes.
A63. The device system, method of measuring, system, training method or system, classification method or system, model, use or training data set of any of the preceding clauses, wherein each of said input measurements (IM) or labelled training input measurements (LTIM) is based on spectroscopy measurement (ME) of at least 20 different wavelengths, such as least 50 different wavelengths, preferably at least 100 different wavelengths, for example at least 150 different wavelengths, such as at least 250 different wavelengths.
A64. The device system, method of measuring, system, training method or system, classification method or system, model, use or training data set of any of the preceding clauses, wherein the at least 20 different wavelengths are in the infrared IR range, such as selected from one or more of
the mid infrared MIR range, for example the spectroscopic fingerprint region, the functional group region, or a combination thereof;
the near infrared NIR range, the short wavelength infrared SWIR range, or a combination thereof;
the medium wavelength infrared MWIR range, the long wavelength infrared LWIR range, or a combination thereof; or
the range of 8,000 nm to 10,500 nm, the range of 5,500 nm to 8,000 nm, the range of 2,500 nm to 20,000 nm, the range of 4,000 nm to 12,000 nm, the range of 5,500 nm to 10,500 nm, or combination thereof.
A65. The device system, method of measuring, system, training method or system, classification method or system, model, use or training data set of any of the preceding clauses, wherein the spectroscopy measurements (ME) are obtained by attenuated total reflectance ATR spectroscopy, preferably single-bounce ATR, preferably using a tunable pyroelectric detector to obtain measurements at different wavelengths.
A66. The device system, method of measuring, system, training method or system, classification method or system, model, use or training data set of any of the preceding clauses, wherein the spectroscopy measurements (ME) are obtained by multispectral imaging or hyperspectral imaging.
A67. The device system, method of measuring, system, training method or system, classification method or system, model, use or training data set of any of the preceding clauses, wherein the spectroscopy measurements (ME) are obtained within a distance of less than 10 μm, such as less than 5 μm, such as less than 4 or 3 or 2 or 1 μm, for example less than 0.5 or 0.1 μm, preferably during contact in at least one contact spot, more preferably a plurality of contact spots, with the surface (SU) comprising a cured coat.
A68. The device system, method of measuring, system, training method or system, classification method or system, model, use or training data set of any of the preceding clauses, wherein the spectroscopy measurements (ME) are obtained while maintaining a pressure of a spectroscopy measurement device against said surface (SU) of at least 5 kg, such as at least 10 kg, preferably at least 20 kg, such as at least about 10,000 kPa, such as at least 25,000 kPa, preferably at least about 50,000 kPa, for example at least 100 kgf/cm², such as at least 250 kgf/cm² or at least 500 kgf/cm².
A69. The device system, method of measuring, system, training method or system, classification method or system, model, use or training data set of clause A68, wherein the pressure of a spectroscopy measurement device against said surface (SU) is maintained by means of a magnetic switchable device (MSD).
A70. The device system, method of measuring, system, training method or system, classification method or system, model, use or training data set of any of the preceding clauses, wherein the spectroscopy measurements (ME) are obtained at a distance of at least 10 μm, such as at least 1 mm, 5 mm, 1 cm, 2.5 cm or 5 cm, for example at least 50 cm or 1 meter, from the surface (SU) comprising a cured coat.
A71. The device system, method of measuring, system, training method or system, classification method or system, model, use or training data set of any of the preceding clauses, wherein said classification model (CM) is a supervised classification model.
A72. The device system, method of measuring, system, training method or system, classification method or system, model, use or training data set of any of the preceding clauses, wherein said classification model (CM) comprises a nonlinear model.
A73. The device system, method of measuring, system, training method or system, classification method or system, model, use or training data set of any of the preceding clauses, wherein said classification model (CM) comprises at least two different types of activation functions (AF).
A74. The device system, method of measuring, system, training method or system, classification method or system, model, use or training data set of any of the preceding clauses, wherein said classification model (CM) comprises at least a SoftMax activation function.
A75. The device system, method of measuring, system, training method or system, classification method or system, model, use or training data set of any of the preceding clauses, wherein said classification model (CM) comprises at least a ReLu type activation function.
A76. The device system, method of measuring, system, training method or system, classification method or system, model, use or training data set of any of the preceding clauses, wherein said classification model (CM) is a neural network.
A77. The device system, method of measuring, system, training method or system, classification method or system, model, use or training data set of any of the preceding clauses, wherein said classification model (CM) is a convolutional neural network (CNNCM).
A78. The device system, method of measuring, system, training method or system, classification method or system, model, use or training data set of any of the preceding clauses, wherein said classification model (CM) comprises a feature extraction module (FEM).
A79. The device system, method of measuring, system, training method or system, classification method or system, model, use or training data set of any of the preceding clauses, wherein said classification model (CM) comprises:
a feature extraction module (FEM) configured to receive training input measurements (TIM) and to generate a feature extraction output (FEO) based on said training input measurements (TIM); and
a classification module (CLM) configured to classify based on the feature extraction output (FEO) of the feature extraction module (FEM).
A80. The device system, method of measuring, system, training method or system, classification method or system, model, use or training data set of any of the preceding clauses, wherein training of said classification model (CM) comprises optimizing a cost function (CF), and wherein said cost function (CF) is based on categorical cross entropy.
A81. The device system, method of measuring, system, training method or system, classification method or system, model, use or training data set of any of the preceding clauses, wherein parameters (CMP) of the classification model (CM) may be optimized based on grid and/or random search for optimal parameters and/or based on cross validation.
A82. The device system, method of measuring, system, training method or system, classification method or system, model, use or training data set of any of the preceding clauses, wherein said classification model (CM) comprises one or more from the list comprising: a gradient boosting model, a decision tree, a support vector machine, a neural network, a Bayesian based model.
A83. The device system, method of measuring, system, training method or system, classification method or system, model, use or training data set of any of the preceding clauses, wherein said classification model (CM) comprises an XGboost model.
A84. The device system, method of measuring, system, training method or system, classification method or system, model, use or training data set of any of the preceding clauses, wherein said classification model (CM) is pretrained based on transfer learning.
A85. The device system, method of measuring, system, training method or system, classification method or system, model, use or training data set of any of the preceding clauses, wherein a size of the training input measurements (TIM) and/or said input measurements (IM) is reduced based on dimensionality reduction and/or based on feature selection.
A86. The device system, method of measuring, system, training method or system, classification method or system, model, use or training data set of any of the preceding clauses, wherein the dimensionality reduction and/or feature selection is based on one or more from the list comprising: principle component analysis, elastic net.
A87. The device system, method of measuring, system, training method or system, classification method or system, model, use or training data set of any of the preceding clauses, wherein said training input measurements (TIM) and/or said input measurements (IM) are smoothed.
A88. The device system, method of measuring, system, training method or system, classification method or system, model, use or training data set of any of the preceding clauses, wherein said training input measurements (TIM) and/or said input measurements (IM) are smoothed using a Savitzky-Golay filter.
A89. The device system, method of measuring, system, training method or system, classification method or system, model, use or training data set of any of the preceding clauses, wherein said step of training comprises steps of
establishing a test data set (TED) on the basis of said labelled training input measurements (LTIM);
generating test classification outputs (TEO) using said test data set (TED) and said trained classification model (TCM);
evaluating said test classification outputs (TEO) on the basis of test success criteria (TEC) to determine a trained classification model quality (TCMQ);
selecting on the basis of said trained classification model quality (TCMQ) to repeat or not repeat said step of training.
A90. The device system, method of measuring, system, training method or system, classification method or system, model, use or training data set of any of the preceding clauses, wherein the trained classification model (TCM) is updated by re-training or transfer learning according to one or more update trigger from the list of: receipt of new training input measurements (TIM), such as when at least 10, 50, 100, 200 or 1000 new training input measurements (TIM) are received, expiry of an update deadline, such as after 1 week, after 2 weeks, after 1 month, after 3 months, after 6 months, or after 1 year, establishment of a new surface characteristic class (SCC) or modification of an existing surface characteristic class (SCC).
A91. The device system, method of measuring, system, training method or system, classification method or system, model, use or training data set of any of the preceding clauses, wherein metadata relating to said surface (SU), such as asset type, asset identification or most recently applied coating system, is assigned to said representation of said measurement (MER).
A92. The device system, method of measuring, system, training method or system, classification method or system, model, use or training data set of any of the preceding clauses, wherein metadata relating to geolocation, weather, temperature, humidity, light or operator (DO), or information about periods where said communication module (DCM) has been prevented from establishing said communication channel, is assigned to said representation of said measurement (MER).
A93. The device system, method of measuring, system, training method or system, classification method or system, model, use or training data set of any of the preceding clauses, wherein the step of training said classification model (CM) is performed by a cloud computing system (CD).
A94. The device system, method of measuring, system, training method or system, classification method or system, model, use or training data set of any of the preceding clauses, wherein the step of classifying said surface (SU) is performed by a cloud computing system (DC).
A95. The device system, method of measuring, system, training method or system, classification method or system, model, use or training data set of any of the preceding clauses, wherein said trained classification model (TCM) is transferred from said cloud computing system (DC) to one or more of said surface characterizing device systems (SCD), such as a smart device (SD) thereof, for performing said step of classifying a surface (SU) locally.
A96. The device system, method of measuring, system, training method or system, classification method or system, model, use or training data set of any of the preceding clauses, wherein said plurality of predefined surface characteristic classes (SCC) further comprises one or more classes selected from a group of filler and pigment classes, a group of contaminant classes, a group of contamination state classes, a group of degradation type classes, a group of degradation state classes, or a combination thereof.
A97. The device system, method of measuring, system, training method or system, classification method or system, model, use or training data set of any of clause A96, wherein said group of filler classes comprise one or more class from the list of carbonates such as: calcium carbonate, calcite, dolomite (=calcium/magnesium carbonate), magnesium silicate/carbonate, polycarbonate, calcined grades, surface treated grades, and mixtures thereof; silicates such as: Aluminium silicate (kaolin, china clay), Magnesium silicate (talc, talc/chlorite), Potassium Aluminium silicate (plastorite, glimmer), Potassium Sodium Aluminium silicate (nepheline syenite), Calcium silicate (wollastonite), Aluminium silicate (bentonite), phyllo silicate (mica); oxides such as: Silicon dioxide such as quartz, diatomite, and metal oxides such as calcium oxide, aluminium oxide; hydroxides/hydrates such as: Aluminium hydroxide, Aluminium trihydrate, Sulphates: barium sulphate; and other fillers such as: Barium metaborate, silicon carbide, Perlite (volcanic glass), glass spheres (solid and hollow), glass flakes, glass and silicate fibres, organic fibres, polyvinylidene chloride acrylonitrile and polystyrene acrylate.
A98. The device system, method of measuring, system, training method or system, classification method or system, model, use or training data set of clause A96 or A97, wherein said group of pigment classes comprise one or more class from the list of zinc oxide, zinc containing phosphate and polyphosphate, aluminium containing phosphate, zinc borate, graphite, carbon black oxide, coated mica, fluorescent pigments, cuprous oxide, aluminium paste pigment (leafing and non-leafing type), metallic pigments, zinc dust, organic pearl pigment, ammonium polyphosphate, coloured silica sand, polyacrylic acid/calcium carbonate, azo-, phthalocyanine and anthraquinone derivatives (organic pigments) and titanium dioxide (titanium(IV) oxide).
A99. The device system, method of measuring, system, training method or system, classification method or system, model, use or training data set of any of the clauses A96-A98, wherein said group of contaminant classes comprise one or more class from the list of water absorption, contaminant absorption, corrosive substances and organic material absorption.
A100. The device system, method of measuring, system, training method or system, classification method or system, model, use or training data set of any of the clauses A96-A99, wherein said group of degradation type classes comprise one or more class from the list of chemical change classes such as chemical decomposition or chemical oxidation; and physical change classes such as structural changes, phase changes.
A101. The device system, method of measuring, system, training method or system, classification method or system, model, use or training data set of any of the preceding clauses, wherein the training method is for training a classification model for characterizing a surface comprising a cured coat in a surface characterization system (SCS) comprising a fleet (FT) of decentral surface characterizing device systems (SCD) and a central training system (TS), and wherein the training method comprises steps of a first subset (FT1) of said fleet (FT) of decentral surface characterizing device systems (SCD) establishing training input measurements (TIM) based on measurements (ME) of training surfaces (TSU) comprising a cured coat; said training input measurements (TIM) being transmitted to said central training system (TS); a second subset (FT2) of said fleet (FT) of decentral surface characterizing device systems (SCD) establishing input measurements (IM) based on measurements (ME) of surfaces (SU) comprising a cured coat; and said central training system (TS) training a classification model (CM) based on said training input measurements (TIM) received from said first subset (FT1) of said fleet (FT) to provide a trained classification model (TCM).
A102. The device system, method of measuring, system, training method or system, classification method or system, model, use or training data set of any of the preceding clauses, wherein an operating method of the surface characterization system (SCS) comprising a fleet (FT) of decentral surface characterizing device systems (SCD), a central training system (TS) and a classification system (CS), comprises steps of: providing the trained classification model (TCM) by the training method; and classifying surfaces (SU) comprising a cured coat based on input measurements (IM) acquired by surface characterizing device systems SCD of said second subset (FT2) of said fleet (FT) using said trained classification model (TCM) to produce classification outputs (CO), the input measurements (IM) being based on measurements (ME) of the surfaces (SU) comprising a cured coat
A103. A surface characterization system (SCS) configured to characterize surfaces (SU) comprising a cured coat,
the surface characterization system (SCS) comprising a fleet (FT) of decentral surface characterizing device systems (SCD), said fleet (FT) comprising a first subset (FT1) of said fleet (FT) of decentral surface characterizing device systems (SCD) configured to establish training input measurements (TIM) based on measurements (ME) of training surfaces (TSU) comprising a cured coat; and a second subset (FT2) of said fleet (FT) of decentral surface characterizing device systems (SCD) configured to establish input measurements (IM) based on measurements (ME) of said surfaces (SU) comprising a cured coat;
the surface characterization system (SCS) comprising a central training system (TS) comprising a classification model (CM) and a training module (TM) configured to train said classification model (CM) based on said training input measurements (TIM) from said first subset (FT1) of said fleet (FT) to provide a trained classification model (TCM); and
the surface characterization system (SCS) comprising a classification system (CS) comprising a classifier (C) configured to classify said surfaces (SU) based on said input measurements (IM) from said second subset (FT2) of said fleet (FT) using said trained classification model (TCM) to produce classification outputs (CO).
A104. The surface characterizing system (SCS) of clause A103, wherein the surface characterizing device systems (SCD) are surface characterizing device systems according to any of the clause A1-A43.
A105. The device system, method of measuring, system, training method or system, classification method or system, model, use or training data set of any of the preceding clauses, wherein the classification method of characterizing surfaces (SU) comprising a cured coat comprises steps of establishing by a central training system (TS) a trained classification model (TCM) based on training input measurements (TIM), the training input measurements (TIM) being based on measurements (ME) of training surfaces (TSU) comprising a cured coat acquired by a first subset (FT1) of a fleet (FT) of decentral surface characterizing device systems (SCD); acquiring by a second subset (FT2) of said fleet (FT) of decentral surface characterizing device systems (SCD) input measurements (IM) based on measurements (ME) of said surfaces (SU) comprising a cured coat; and classifying said surfaces (SU) based on said input measurements (IM) using said trained classification model (TCM) to produce classification outputs (CO).
A106. A fleet (FT) of decentral surface characterizing device systems (SCD), said fleet (FT) comprising a first subset (FT1) and a second subset (FT2), wherein
   said first subset (FT1) of said fleet (FT) of decentral surface characterizing device systems (SCD) being configured to establish training input measurements (TIM) based on measurements (ME) of training surfaces (TSU) comprising a cured coat and transmit said training input measurements (TIM) to a central training system (TS); and
   said second subset (FT2) of said fleet (FT) of decentral surface characterizing device systems (SCD) being configured to establish input measurements (IM) based on measurements (ME) of surfaces (SU) comprising a cured coat and transmit said input measurements (IM) to a classification system (CS) comprising a classifier (C).
A107. The fleet (FT) of clause A106, wherein the surface characterizing device systems (SCD) are surface characterizing device systems according to any of the clause A1-A43.
A108. The device system, method of measuring, system, training method or system, classification method or system, model, use, training data set, surface characterizing system or fleet of any of the clauses A101-A107, wherein said training system (TS) trains said classification model (CM) based on said training input measurement (TIM) from said first subset (FT1) and not from said second subset (FT2) of said fleet (FT).
A109. The device system, method of measuring, system, training method or system, classification method or system, model, use, training data set, surface characterizing system or fleet of any of the clauses A101-A108, wherein said classification outputs (CO) are provided to respective of said surface characterizing device systems (SCD) of said second subset (FT2) of said fleet (FT).
A110. The device system, method of measuring, system, training method or system, classification method or system, model, use, training data set, surface characterizing system or fleet of any of the clauses A101-A109, wherein said decentral surface characterizing device systems (SCD) of said second subset (FT2) comprise said classification system (CS), and wherein said classification system (CS) is configured to classify said surfaces (SU) based on said input measurements (IM) using a trained classification model (TCM) received from said central training system (TS) to produce classification outputs (CO).
A111. The device system, method of measuring, system, training method or system, classification method or system, model, use, training data set, surface characterizing system or fleet of any of the clauses A101-A110, wherein said fleet (FT) comprises two or more surface characterizing device systems (SCD), such as at least 5, 10, 20 or 100 surface characterizing device systems (SCD).
A112. The device system, method of measuring, system, training method or system, classification method or system, model, use, training data set, surface characterizing system or fleet of any of the clauses A101-A111, wherein said first subset (FT1) comprises two or more surface characterizing device systems (SCD), such as at least 5, 10, 20 or 100 surface characterizing device systems (SCD).
A113. The device system, method of measuring, system, training method or system, classification method or system, model, use, training data set, surface characterizing system or fleet of any of the clauses A101-A112, wherein said second subset (FT2) comprises one or more surface characterizing device systems (SCD), such as at least 2, 5, 10, 20 or 100 surface characterizing device systems (SCD).
A114. The device system, method of measuring, system, training method or system, classification method or system, model, use, training data set, surface characterizing system or fleet of any of the clauses A101-A113, wherein said first subset (FT1) and said second subset (FT2) are different.
A115. The device system, method of measuring, system, training method or system, classification method or system, model, use, training data set, surface characterizing system or fleet of any of the clauses A101-A114, wherein said subsets (FT1, FT2) overlap by at least one, but preferably not all, such as for example 1, 2, 5 or 10, surface characterizing device systems (SCD) providing both training input measurements (TIM) and input measurements (IM).
A116. The device system, method of measuring, system, training method or system, classification method or system, model, use, training data set, surface characterizing system or fleet of any of the clauses A101-A115, wherein the surface characterizing device systems (SCD) of both subsets (FT1, FT2) of the subsets are identical or comprises identical sensors (DS) or are configured to perform measurements (ME) of the same type, such as spectroscopy, or single-bounce ATR spectroscopy.
A117. The device system, method of measuring, system, training method or system, classification method or system, model, use, training data set, surface characterizing system or fleet of any of the clauses A101-A116, wherein the belonging of a surface characterizing device system (SCD) to the first subset (FT1) is evaluated for each measurement (ME), for each surface (SU), for each coated structure (CTS), for each change of operator (DO) or for each predefined time duration such as 1 day, 1 week, 2 weeks, 1 months, 3 months, 6 months or 1 year.

### LIST OF REFERENCE SIGNS

AF activation function
BI bias
BSS base structure
C classifier
CB1-5 convolution block
CBN coat binder
CBN′ coat binder derivative
CF cost function
CFP coat fillers and pigments
CFP′ coat fillers and pigments derivative
CL11-53 convolutional layer
CLM classification module
CM classification model
CMO classification model optimizer
CMP classification model parameters
CMS1-3 classification method steps
CNNCM convolutional neural network classification model
CO classification output
COD classification output determiner
CS classification system
CTS coated structure
DAS accelerometer sensor
DB battery
DC cloud
DCC communication channel
DCM communication module
DCS camera sensor
DD visual output device, such as an LED or a display
DE infrared emitter
DEC emitted IR centerline
DED emitter driver
DFL flash light
DH device handle
DHIC hyperspectral imaging camera
DIFL infrared flash light
DL dense layer
DLS light sensor
DM memory
DMB measurement buffer
DO device operator
DP processor
DPR prism/crystal
DPS pressure sensor
DR drone or robot
DRC reflected IR centerline
DS sensor
DSD sensor driver
DTS temperature sensor
DUI user interface
ECM error calculation module
FEM feature extraction module
FEO feature extraction output
FL flattening layer
FT fleet of decentral surface characterizing device systems
FT1, FT2 first and second subsets of the fleet
FTD dual role devices
HL hidden layers
IL input layer
IM input measurement
IMR input measurement receiver
LTIM labelled training input measurement
MD measurement device
ME measurement
MER representation of a measurement
MLPM multilayer perceptron model
MLPB multilayer perceptron block
MSD magnetic switchable device
NO neuronal output
OL output layer
OLAF output layer activation function
PL1-5 pooling layer
SCC surface characteristic class
SCD surface characterizing device system
SCS surface characterization system
SD smart device
SU surface comprising a cured coat
SUC surface contamination
SUL surface layer
SUO surface outside
SUT surface thickness
TCM trained classification model
TCMQ trained classification model quality
TCO training classification output
TE training error
TEC test success criteria
TED test data set
TEO test classification output
TDS training data set
TDSG training data set generator
TIL training input label
TIM training input measurement
TIML training input measurement labeller
TIMR training input measurement receiver
TM training module
TMPO training model parameter optimizer
TMS1-6 training method steps
TS training system
TSU training surface comprising a cured coat
UCMP updated classification model parameters

1-117. (canceled)

118. A surface characterizing device system, comprising
at least one sensor configured to acquire a measurement of at least one characteristic of a surface comprising a cured coat;
a processor and memory configured to establish a representation of said measurement;
a visual output device configured to indicate at least a measurement or connection status;
a user interface configured to control measurement; and
a communication module configured to establish a communication channel with a cloud and transmit said representation of said measurement via said communication channel;
wherein said surface characterizing device system is a portable system and being battery powered;
wherein said surface characterizing device system comprises a measurement buffer and is configured to temporarily store said representation of said measurement in said measurement buffer when said communication module is prevented from establishing said communication channel and transmit said temporarily stored representation upon reestablishment of said communication channel;
wherein said temporarily stored representation is one of: a training input measurement or a labelled training input measurement for a training system configured to train a classification model for characterizing a surface comprising a cured coat, or an input measurement for a classification system configured to classify said surface comprising a cured coat.

119. The surface characterizing device system of claim 118, wherein the surface characterizing device system is a distributed system comprising a portable measurement device and portable smart device;
wherein the measurement device comprises at least one of said at least one sensor;
wherein the smart device comprises at least said communication module configured to establish said communication channel with said cloud; and
wherein the measurement device is communicatively coupled to said smart device to transfer said measurement or said representation.

120. The surface characterizing device system of claim 119, wherein the smart device is configured to assign to the representation of said measurement one or more of further data from a sensor of said smart device, training input labels, metadata obtained by the smart device, information about periods where said communication module has been prevented from establishing said communication channel.

121. The surface characterizing device system of claim 118, wherein said measurement buffer is configured to temporarily store at least two of said representations of said measurements.

122. The surface characterizing device system of claim 118, wherein said sensor is configured for making spectroscopy measurements.

123. The surface characterizing device system of claim 122, wherein the sensor is configured to obtain the spectroscopy measurements by attenuated total reflectance ATR spectroscopy at wavelengths in the range of 5,500 nm to 8,000 nm.

124. The surface characterizing device system of claim 118, wherein said surface characterizing device system comprises at least one infrared emitter, at least one prism and at least one sensor configured for attenuated total reflection spectroscopy of said surface comprising a cured coat to obtain a spectroscopy measurement.

125. The surface characterizing device system of claim 124, wherein said at least one infrared emitter, said at least one prism and said at least one sensor are configured to perform spectroscopy in a range of at least one of the following: the long wavelength infrared LWIR range, the range of 8,000 nm to 10,500 nm, the medium wavelength infrared MWIR range, the range of 5,500 nm to 8,000 nm, the range of 5,500 nm to 10,500 nm.

126. The surface characterizing device system of claim 118, wherein said surface characterizing device system comprises one or more of a pressure sensor and an accelerometer sensor.

127. The surface characterizing device system of claim 118, wherein said surface characterizing device system is configured to perform a filtering or pre-validation of measurements and determine whether to temporarily store said representation of said measurement in said measurement buffer based on a result of said filtering or pre-validation.

128. The surface characterizing device system of claim 118, wherein said surface characterizing device system comprises a trained classification model for characterizing a surface comprising a cured coat, and wherein said surface characterizing device system is configured to perform a classification based on said representation of said measurement and determine whether to temporarily store said representation on the basis of a result of said classification.

129. The surface characterizing device system of claim 128, wherein said trained classification model is configured to classify said surface into at least one predefined surface characteristic class from a plurality of predefined surface characteristic classes based on said measurement, the surface characteristic classes comprising one or more classes selected from a group of binder system classes, a group of coat manufacturer classes, a group of coating product classes, or a combination thereof.

130. Method of measuring a characteristic of a surface comprising a cured coat; the method comprising steps of:
providing a surface characterizing device system in a first environment where communication with a cloud external to said first environment is prevented;
using the surface characterizing device system to acquire a measurement of at least one characteristic of a surface comprising a cured coat;
storing a representation of said measurement temporarily in a measurement buffer of said surface characterizing device system;
providing said surface characterizing device system in a second environment;
establishing a communication channel between said surface characterizing device system and said cloud; and
transmitting said temporarily stored representation of said measurement to said cloud.

131. Computer-implemented classification method of characterizing a surface comprising a cured coat, the method comprising steps of:
providing a trained classification model by
receiving training input measurements from a surface characterizing device system based on measurements of training surfaces comprising a cured coat;
generating labelled training input measurements by individually labelling said training input measurements in accordance with a plurality of predefined surface characteristic classes;
establishing a training data set on the basis of said labelled training input measurements;
providing a classification model;
training said classification model based on said training data set to provide a trained classification model;
receiving an input measurement from a surface characterizing device system based on a measurement of said surface comprising a cured coat; and
classifying said surface into at least one of said predefined surface characteristic classes based on said input measurement using said trained classification model to produce a classification output;
wherein said surface characterizing device system comprises at least one sensor configured to acquire a measurement of at least one characteristic of a surface comprising a cured coat; a processor and memory configured to establish a representation of said measurement; a visual output device configured to indicate at least a measurement or connection status; a user interface configured to control measurement; and a communication module configured to establish a communication channel with a cloud and transmit said representation of said measurement via said communication channel; wherein said surface characterizing device system is a portable system and being battery powered; and wherein said surface characterizing device system comprises a measurement buffer and is configured to temporarily store said representation of said measurement in said measurement buffer when said communication module is prevented from establishing said communication channel and transmit said temporarily stored representation upon reestablishment of said communication channel.

132. The classification method of claim 131, wherein said plurality of predefined surface characteristic classes is a plurality of classes selected from the group of binder system classes; wherein said group of binder system classes comprises one or more class from the list of acrylic, epoxy, polyaspartic, polyurethane, polysiloxane, alkyd, silicate, silicone, polyurea, rosin, vinyl copolymers, polydimethylsiloxane, and hybrid technologies.

133. The classification method of claim 131, wherein each of said input measurements or labelled training input measurements is based on spectroscopy measurement obtained by attenuated total reflectance ATR spectroscopy of at least 20 different wavelengths; wherein the at least 20 different wavelengths are in the infrared IR range, selected from one or more of:
the mid infrared MIR range, the near infrared NIR range, the short wavelength infrared SWIR range, the medium wavelength infrared MWIR range, the long wavelength infrared LWIR range, the range of 8,000 nm to 10,500 nm, the range of 5,500 nm to 8,000 nm, the range of 2,500 nm to 20,000 nm, the range of 4,000 nm to 12,000 nm, the range of 5,500 nm to 10,500 nm, or combination thereof.

134. The classification method of claim 131, wherein the spectroscopy measurements are obtained while maintaining a pressure of a spectroscopy measurement device against said surface of at least one of at least 5 kg, at least about 10,000 kPa, at least 100 kgf/cm².

135. The classification method of claim 131, wherein said classification model is a supervised classification model and a convolutional neural network.

136. The classification method of claim 131, wherein the trained classification model is updated by re-training or transfer learning according to one or more update trigger from the list of: receipt of new training input measurements, expiry of an update deadline, establishment of a new surface characteristic class, modification of an existing surface characteristic class.

137. The classification method of claim 131, wherein said trained classification model is transferred from said cloud computing system to said surface characterizing device system for performing said step of classifying a surface locally.
